(19) United States
(12) Patent Application Publication
(10) Pub. No.: US 2022/0017541 A1
(43) Pub. Date: Jan. 20, 2022

(54) INHIBITORS OF ARGINASE

(71) Applicant: Black Belt Tx Ltd, Stevenage Hertfordshire (GB)

(72) Inventors: Sebastian BELMAR, Santiago (CL); Jennifer ALFARO, Santiago (CL); Gonzalo Esteban NÚÑEZ VASQUEZ, Santiago (CL); Sebastian BERNALES, Piedmont, CA (US); Brahmam PUJALA, Greater Noida (IN); Dayanand PANPATIL, Noida (IN); Pasha KHAN, Greater Noida (IN); Sarvajit CHAKRAVARTY, Edmond, OK (US); Gonzalo Andrés URETA DÍAZ, Santiago (CL)

(21) Appl. No.: 17/296,150

(22) PCT Filed: Nov. 21, 2019
(86) PCT No.: PCT/EP2019/082158

§ 371 Date: May 21, 2021

Related U.S. Application Data
(60) Provisional application No. 62/770,682, filed on Nov. 21, 2018.

(51) Int. Cl. C07F 5/02; A61P 35/00

(52) U.S. Cl. CPC C07F 5/025; A61P 35/00

(57) ABSTRACT

Compounds and compositions as inhibitors of arginase are provided. They may find use as therapeutic agents for the treatment of diseases or conditions associated with expression or activity of arginase.

### CROSS-REFERENCE TO RELATED APPLICATIONS

This application claims prior benefit of U.S. Provisional Patent Application No. 62/770,682, filed Nov. 21, 2018, the disclosures of which are hereby incorporated herein by reference in their entirety.

### FIELD OF THE INVENTION

This disclosure relates generally to compounds and compositions that may be useful as inhibitors of arginase.

### BACKGROUND

Arginase is a binuclear manganese metalloenzyme that catalyzes the hydrolysis of L-arginine to urea and ornithine (Ash, D. E., J Nutr, 2004. 134: 2760S-2764S; discussion 2765S-2767S). There are two isoforms of arginase, and they are both products of distinct genes that are regulated independently and located on different chromosomes. Arginase I (ARG1) is a cytosolic protein (34.7 kDa) and is dominant in the liver, but also expressed extrahepatically (Maarsingh, H., et al., Br J Pharmacol, 2009. 158(3): p. 652-64). Arginase II (ARG2) is a mitochondrial protein and is expressed in kidney, small intestine, brain, monocytes and macrophages (Wu, G., et al., Biochem J, 1998. 336 (Pt 1): p. 1-17). In addition to producing urea and ornithine, arginase also regulates arginine levels for nitric oxide synthases (NOS).

In humans, arginase is abundantly expressed and/or stored in polymorphonuclear neutrophils (Rotondo, R., et al., Int J Cancer, 2009. 125(4): p. 887-93) and released in response to inflammation. The fundamental mechanism of inflammation-associated immunosuppression is the suppression of the T-cell immune response by arginase-mediated L-arginine depletion in myeloid cells (Brittenden, J., et al., Clin Sci (Lond), 1994. 86(2): p. 123-32). L-arginine deficiency down-regulates expression of T cell receptor (TCR) ζ chain, a key signaling element of the TCR, thereby impairing T cell function (Rodriguez, P. C., et al., J Biol Chem, 2002. 277(24): p. 21123-9). It also has been describe that L-arginine availability modulates the phenotypic and functional properties of NK cells (Lamas, B., et al., Cell Immunol, 2012. 280(2): p. 182-90).

Targeting the arginase and polyamine biosynthetic pathways is now being attempted for the treatment of various diseases such as African sleeping sickness, Chagas disease and leishmaniasis (Heby, O., et al., Amino Acids, 2007. 33(2): p. 359-66). Since both Arginase and NOS compete for the same substrate, arginase downregulates NO synthesis and induces fibrosis (McLarren, K. W., et al., Am J Pathol, 2011. 179(1): p. 180-8) and tissue regeneration (Kavalukas, S. L., et al., Surgery, 2012. 151(2): p. 287-95). Inhibition of arginase prevents the development of diabetic nephropathy (You, H., et al., Am J Physiol Renal Physiol, 2015. 309(5): p. F447-55). Arginase regulates the availability of proline for cell proliferation and collagen deposition in diseases such as asthma (Benson, R. C., et al., J Allergy (Cairo), 2011. 2011: p. 736319) and some cancers (Morris, S. M., Jr., Br J Pharmacol, 2009. 157(6): p. 922-30). Arginase was shown to participate in the suppression of tumor-infiltrating lymphocytes in patients with prostate cancer (Bronte, V., et al., J Exp Med, 2005. 201(8): p. 1257-68), non-small cell lung carcinoma (Rodriguez, P. C., et al., Cancer Res, 2004. 64(16): p. 5839-49) and multiple myeloma (Serafini, P., et al., J Exp Med, 2006. 203(12): p. 2691-702). Plasma samples from cancer patients exhibited elevated Arg1 and reduced L-arginine compared to healthy volunteers (Steggerda, S. M., et al., J Immunother Cancer, 2017. 5(1): p. 101). Arginase activity of neuroblastoma impairs NY-ESO-1 specific TCR and GD2-specific CAR engineered T cell proliferation and cytotoxicity which leads to sub-optimal efficacy of immunotherapeutic approaches (Mussai, F., et al., Cancer Res, 2015. 75(15): p. 3043-53). It was shown that arginase inhibition blocked myeloid cell-mediated suppression of T cell proliferation in vitro and reduced tumor growth in multiple mouse models of cancer.

Given the role of arginase in various pathological states, arginase inhibitors may provide a therapeutic strategy for those diseases or conditions of which arginases are implicated in the processes.

### BRIEF SUMMARY

In one aspect, provided is a compound of the formula (I):

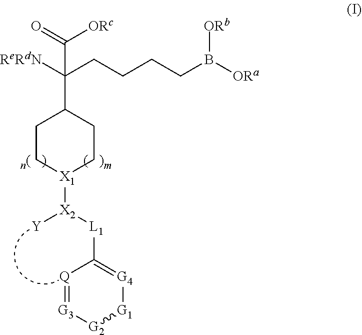

or a pharmaceutically acceptable salt, stereoisomer or tautomer thereof, wherein X₁, X₂, Y, Q, [symbol] , L₁, G₁, G₂, G₃, G₄, [symbol] , m, n, R^{a}, R^{b}, R^{c}, R^{d} and R^{e} are as detailed herein.

In some embodiments, the compound of Formula (I), or a pharmaceutically acceptable salt, stereoisomer or tautomer thereof, is of the Formula (IIa), (IIb), (IIIa), (IIIIb), (IVa), or (IVb) as detailed herein.

In another aspect, provided is a method of treating a disease or condition associated with expression or activity of arginase.

Also provided are pharmaceutical compositions comprising: (A) a compound detailed herein, such as a compound of Formula (I) or a pharmaceutically acceptable salt, stereoisomer or tautomer thereof, or a compound of Formula (IIa), (IIb), (IIIa), (IIIIb), (IVa), or (IVb) or a pharmaceutically acceptable salt, stereoisomer or tautomer thereof; and (B) a pharmaceutically acceptable carrier or excipient. Kits comprising a compound detailed herein or a pharmaceutically acceptable salt, stereoisomer or tautomer thereof and optionally instructions for use are also provided. Compounds as detailed herein or a pharmaceutically acceptable salt, stereoisomer or tautomer thereof are also provided for the manufacture of a medicament for the treatment of a disease or disorder disclosed herein.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃).

“Heterocycle”, “heterocyclic”, or “heterocyclyl” as used herein refers to a saturated or an unsaturated non-aromatic cyclic group having from 1 to 14 annular carbon atoms and from 1 to 6 annular heteroatoms, such as nitrogen, sulfur or oxygen, and the like. A heterocyclic group may have a single ring (e.g., pyrrolidinyl) or multiple condensed rings (e.g., decahydroisoquinolin-1-yl), which condensed rings may or may not be aromatic and which may be carbocylic or contain one or more annular heteroatoms, but which excludes heteroaryl rings. A heterocycle comprising more than one ring may be fused, bridged or spiro, or any combination thereof. In fused ring systems, one or more of the fused rings can be cycloalkyl or aryl, but excludes heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases which can be used to prepared salts include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of an individual. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying recurrence, such as of unwanted cell proliferation.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Unless otherwise stated, “substantially pure” intends a composition that contains no more than 10% impurity, such as a composition comprising less than about 9%, 7%, 5%, 3%, 1%, 0.5% impurity.

It is understood that aspects and variations described herein also include “consisting” and/or “consisting essentially of” aspects and variations.

All references throughout, such as publications, patents, patent applications and published patent applications, are incorporated herein by reference in their entireties.

### Compounds

In one aspect, provided is a compound of Formula (I):

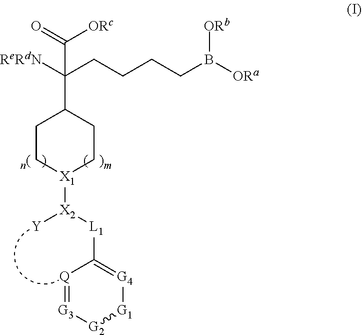

or a pharmaceutically acceptable salt, stereoisomer or tautomer thereof, wherein:
X₁ and X₂ are independently N or CH;
Y is CR¹R², CR¹R²R³, —O—, —OR⁴, —S—, —SR⁵, —NR⁶—, or —NR⁶R⁷;

### Q is C or CR⁸;

[symbol] is absent or C₁-C₄ alkylene, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo;
provided that: (i) [symbol] is C₁-C₄ alkylene, taken together with Y and Q to form a ring,
when Y is CR¹R² and Q is C; and (ii) [symbol] is absent when Y is CR¹R²R³ and Q is CR⁸;
   L₁ is a bond or C₁-C₄ alkylene, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo;
   G₁ is CR^{1a}, C(O), N or NH;
   G₂ is CR^{2a}, C(O), N or NH; [symbol]
   is a single bond or a double bond, provided that: (i) [symbol] is a single bond when G₁ is C(O) and G₂ is NH or when G₂ is C(O) and G₁ is NH; and (ii) [symbol] is a double bond when G₁ is CR^{1a} and G₂ is CR^{2a} or N, and when G₁ is N and G₂ is CR^{2a};
   G₃ is CR^{3a} or N;
   G₄ is CR^{4a} or N;
   m and n are independently 0, 1 or 2;
R^{a} and R^{b} are each independently hydrogen, C₁-C₆ alkyl, or C₃-C₈ cycloalkyl, wherein the C₁-C₆ alkyl and C₃-C₈ cycloalkyl are each independently unsubstituted or substituted with 1, 2, 3, or 4 substituents selected from the group consisting of halogen, C₁-C₆ alkyl, C₃-C₈ cycloalkyl, —CN, —OH, and oxo;
   or R^{a} and R^{b} are taken together with the atoms to which they are attached to form a 5- to 10-membered heterocyclyl, wherein the 5- to 10-membered heterocyclyl is unsubstituted or substituted with 1, 2, 3, or 4 substituents selected from the group consisting of halogen, C₁-C₆ alkyl, C₃-C₈ cycloalkyl, —CN, —OH, and oxo;
R^{c}, R^{d} and R^{e} are each independently hydrogen, C₁-C₆ alkyl, C₃-C₈ cycloalkyl, or C₆-C₁₄ aryl, wherein the C₁-C₆ alkyl, C₃-C₈ cycloalkyl and C₆-C₁₄ aryl are each independently unsubstituted or substituted with 1, 2, 3, or 4 substituents selected from the group consisting of halogen, C₁-C₆ alkyl, C₃-C₈ cycloalkyl, —CN, —OH, and oxo;
R¹, R², R³, R⁴, R⁵, R⁶, R⁷, and R⁸ are each independently hydrogen, halo, C₁-C₆ alkyl, or C₃-C₈ cycloalkyl, wherein the C₁-C₆ alkyl and C₃-C₈ cycloalkyl are each independently unsubstituted or substituted with 1, 2, 3, or 4 substituents selected from the group consisting of halogen, C₁-C₆ alkyl, C₃-C₈ cycloalkyl, —CN, —OH, and oxo;
R^{1a}, R^{2a}, R^{3a} and R^{4a} are each independently hydrogen, halo, —CN, —OR^{f}, NR^{g}R^{h}, C₁-C₆ alkyl, C₂-C₆ alkenyl, C₂-C₆ alkynyl, C₃-C₈ cycloalkyl, or C₆-C₁₄ aryl, wherein the C₁-C₆ alkyl, C₂-C₆ alkenyl, C₂-C₆ alkynyl, C₃-C₈ cycloalkyl and C₆-C₁₄ aryl are each independently unsubstituted or substituted by 1, 2, 3, or 4 groups independently selected from the group consisting of halogen, C₁-C₆ alkyl, C₃-C₈ cycloalkyl, —CN, —OH, and oxo; and
   R^{f}, R^{g}, and R^{h} are each independently hydrogen, C₁-C₆ alkyl, C₃-C₈ cycloalkyl, or C₆-C₁₄ aryl, wherein the C₁-C₆ alkyl, C₃-C₈ cycloalkyl and C₆-C₁₄ aryl are each independently unsubstituted or substituted with 1, 2, 3, or 4 substituents selected from the group consisting of halogen, C₁-C₆ alkyl, C₃-C₈ cycloalkyl, —CN, —OH, and oxo.

In some embodiments, provided is a compound of Formula (I), or a pharmaceutically acceptable salt, stereoisomer or tautomer thereof, wherein at least one of m and n is 1 or 2 when X₂ is N. In some embodiments, provided is a compound of Formula (I), or a pharmaceutically acceptable salt, stereoisomer or tautomer thereof, wherein at least one of m and n is 1 or 2. It is understood that either of these provisos, where applicable, can apply to other formulae detailed herein, such as formulae (IIa), (IIb), (IIIa), (IIIb), (IVa) and (IVb) described below.

In some embodiments, provided is a compound of Formula (IIa) or Formula (IIb):

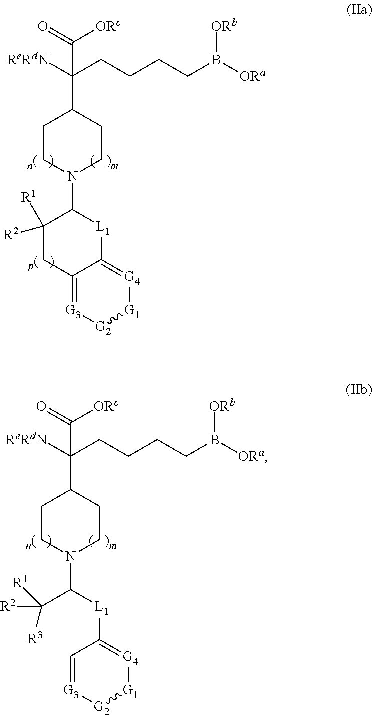

or a pharmaceutically acceptable salt, stereoisomer or tautomer thereof, wherein p is 1 or 2, and G₁, G₂, G₃, G₄, [symbol] , L₁, m, n, R^{a}, R^{b}, R^{c}, R^{d} and R^{e} are as detailed herein.

In some embodiments, provided is a compound of Formula (IIIa) or Formula (IIIb):

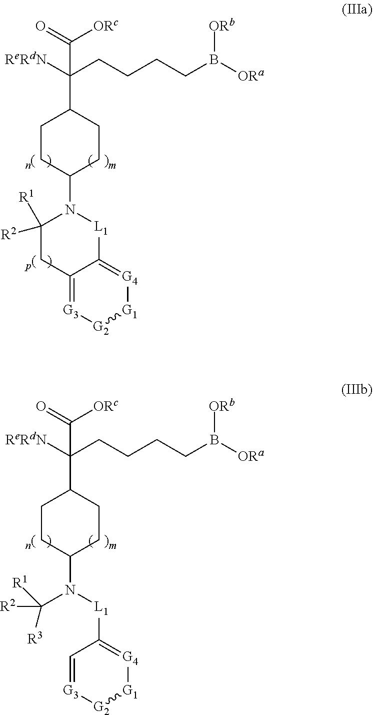

or a pharmaceutically acceptable salt, stereoisomer or tautomer thereof, wherein p is 1 or 2, and G₁, G₂, G₃, G₄, [symbol] , L₁, m, n, R^{a}, R^{b}, R^{c}, R^{d} and R^{e} are as detailed herein.

In some embodiments, provided is a compound of Formula (IVa) or Formula (IVb):

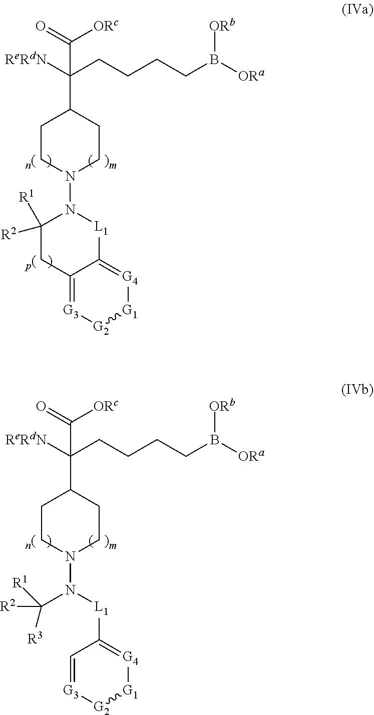

or a pharmaceutically acceptable salt, stereoisomer or tautomer thereof, wherein p is 1 or 2, and G₁, G₂, G₃, G₄, [symbol] , L₁, m, n, R^{a}, R^{b}, R^{c}, R^{d} and R^{e} are as detailed herein.

In one variation is provided a compound of formula (I), or a pharmaceutically acceptable salt, stereoisomer or tautomer thereof, wherein the carbon bearing the —NR^{d}R^{e} and —COOR^{c} moieties is in the “S” configuration. In another variation is provided a compound of the formula (I), or a pharmaceutically acceptable salt, stereoisomer or tautomer thereof, wherein the carbon bearing the —NR^{d}R^{e} and —COOR^{c} moieties is in the “R” configuration. Mixtures of a compound of the formula (I), or a pharmaceutically acceptable salt thereof, are also embraced, including racemic or non-racemic mixtures of a given compound, and mixtures of two or more compounds of different chemical formulae.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to X₁ and X₂ of formula (I) may be combined with every description, variation, embodiment or aspect of G₁, G₂, G₃, G₄, [symbol] m, n, p, R^{a}, R^{b}, R^{c}, R^{d} and/or R^{e} the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae (IIa), (IIb), (IIIa), (IIIb), (IVa), and (IVb) detailed herein, and are equally described, the same as if each and every description, variation, embodiment or aspect were separately and individually listed for all formulae.

In some embodiments of the compound of formula (I), or a pharmaceutically acceptable salt, stereoisomer or tautomer thereof, X₁ is N and X₂ is CH.

In some embodiments of the compound of formula (I), or a pharmaceutically acceptable salt, stereoisomer or tautomer thereof, X₁ is CH and X₂ is N.

In some embodiments of the compound of formula (I), or a pharmaceutically acceptable salt, stereoisomer or tautomer thereof, X₁ is N and X₂ is N.

In some embodiments of the compound of formula (I), or a pharmaceutically acceptable salt, stereoisomer or tautomer thereof, R^{a} and R^{b} are each independently hydrogen, C₁-C₆ alkyl, or C₃-C₈ cycloalkyl, wherein the C₁-C₆ alkyl and C₃-C₈ cycloalkyl are each independently unsubstituted or substituted with 1, 2, 3, or 4 substituents selected from the group consisting of halogen, C₁-C₆ alkyl, C₃-C₈ cycloalkyl, —CN, —OH, and oxo. In some embodiments, R^{a} and R^{b} are taken together with the atoms to which they are attached to form a 5- to 10-membered heterocyclyl, wherein the 5- to 10-membered heterocyclyl is unsubstituted or substituted with 1, 2, 3, or 4 substituents selected from the group consisting of halogen, C₁-C₆ alkyl, C₃-C₈ cycloalkyl, —CN, —OH, and oxo. In some embodiments, R^{a} and R^{b} are taken together with the atoms to which they are attached to form a 5-membered heterocyclyl, wherein the 5-membered heterocyclyl is unsubstituted or substituted with 1, 2, 3, or 4 substituents selected from the group consisting of halogen, C₁-C₆ alkyl, C₃-C₈ cycloalkyl, —CN, —OH, and oxo. In one embodiments, R^{a} and R^{b} are taken together with the atoms to which they are attached to form

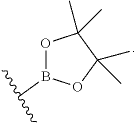 [SMILES: *B1OC(C)(C)C(C)(C)O1]

In some embodiments of the compound of formula (I), or a pharmaceutically acceptable salt, stereoisomer or tautomer thereof, R^{c}, R^{d} and R^{e} are each independently hydrogen, C₁-C₆ alkyl, C₃-C₈ cycloalkyl, or C₆-C₁₄ aryl, wherein the C₁-C₆ alkyl, C₃-C₈ cycloalkyl and C₆-C₁₄ aryl are each independently unsubstituted or substituted with 1, 2, 3, or 4 substituents selected from the group consisting of halogen, C₁-C₆ alkyl, C₃-C₈ cycloalkyl, —CN, —OH, and oxo. In some embodiments, R^{c}, R^{d} and R^{e} are each independently hydrogen, or C₁-C₆ alkyl. In some embodiments, R^{e} and R^{d} are hydrogen and R^{c} is C₁-C₆ alkyl. In some embodiments, R^{c} and R^{d} are hydrogen and R^{e} is C₁-C₆ alkyl.

In some embodiments of the compound of formula (I), or a pharmaceutically acceptable salt, stereoisomer or tautomer thereof, Y is CR¹R², CR¹R²R³, —O—, —OR⁴, —S—, —SR⁵, —NR⁶—, or —NR⁶R⁷. In certain embodiments, Y is CR¹R² or CR¹R²R³.

In some embodiments of the compound of formula (I), or a pharmaceutically acceptable salt, stereoisomer or tautomer thereof, Q is C. In other embodiments, Q is CR⁸.

In some embodiments of the compound of formula (I), or a pharmaceutically acceptable salt, stereoisomer or tautomer thereof, [symbol] is absent when Y is CR¹R²R³ and Q is CR⁸.

In other embodiments of the compound of formula (I), or a pharmaceutically acceptable salt, stereoisomer or tautomer thereof, [symbol] is C₁-C₄ alkylene, taken together with Y and Q to form a ring, when Y is CR¹R² and Q is C, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo. In some embodiments, [symbol] is C₁-C₄ alkylene which is substituted with 1, 2, 3, or 4 substituents selected from the group consisting of halogen, C₁-C₆ alkyl, C₃-C₈ cycloalkyl, —CN, —OH, and oxo. In some embodiments, [symbol] is C₁-C₄ alkylene which is substituted with 1, 2, 3, or 4 substituents selected from the group consisting of halogen and C₁-C₆ alkyl. In some embodiments, [symbol] is C₁-C₄ alkylene which is substituted with 1, 2, 3, or 4 C₁-C₆ alkyl. In some embodiments, [symbol] is C₁-C₄ alkylene which is substituted with 1, 2, 3, or 4 methyl. In other embodiments, [symbol] is —CH₂—, —CH₂—CH₂—, —CH₂—CH₂—CH₂—, or —CH₂—CH₂—CH₂—CH₂—. In some embodiments, [symbol] is —CH₂—. In yet another embodiment, [symbol] is —CH₂—CH₂—.

In some embodiments of the compound of formula (I), or a pharmaceutically acceptable salt, stereoisomer or tautomer thereof, L₁ is a bond or C₁-C₄ alkylene, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo. In some embodiments, L₁ is C₁-C₂ alkylene, wherein the C₁-C₂ alkylene is unsubstituted or substituted with 1, 2, 3, or 4 substituents selected from the group consisting of halogen, C₁-C₆ alkyl, C₃-C₈ cycloalkyl, —CN, —OH, and oxo. In one variation, L₁ is a bond. In another variation, L₁ is methylene. In yet another variation, L₁ is ethylene.

In some embodiments of the compound of formula (I), or a pharmaceutically acceptable salt, stereoisomer or tautomer thereof, [symbol] is a single bond when G₁ is C(O) and G₂ is NH or when G₂ is C(O) and G₁ is NH.

In some embodiments of the compound of formula (I), or a pharmaceutically acceptable salt, stereoisomer or tautomer thereof, [symbol] is a double bond when G₁ is CR^{1a} and G₂ is CR^{2a} or N, and when G₁ is N and G₂ is CR^{2a}.

In some embodiments of the compound of formula (I), or a pharmaceutically acceptable salt, stereoisomer or tautomer thereof, R^{1a} and R^{2a} are each independently hydrogen, halo, —CN, —OR^{f}, NR^{g}R^{h}, C₁-C₆ alkyl, or C₆-C₁₄ aryl. In some embodiments, R^{1a} and R^{2a} are each independently hydrogen, halo, C₁-C₆ alkyl, or C₆-C₁₄ aryl. In some embodiments, R^{1a} and R^{2a} are each independently hydrogen, or halo. In some embodiments, R^{1a} and R^{2a} are each independently —H, —F, —Cl, —Br, —CH₃, —CH₂F, —CF₃, —CH₂OH, —CN, or —NH₂.

In some embodiments of the compound of formula (I), or a pharmaceutically acceptable salt, stereoisomer or tautomer thereof, G₃ is CR^{3a}. In some embodiments, R^{3a} is hydrogen, halo, —CN, —OR^{f}, NR^{g}R^{h}, C₁-C₆ alkyl, or C₆-C₁₄ aryl. In some embodiments, R^{1a} is hydrogen, halo, C₁-C₆ alkyl, or C₆-C₁₄ aryl. In some embodiments, R^{3a} is hydrogen, or halo. In some embodiments, R^{3a} is —H, —F, —Cl, —Br, —CH₃, —CH₂F, —CF₃, —CH₂OH, —CN, or —NH₂. In other embodiments, G₃ is N.

In some embodiments of the compound of formula (I), or a pharmaceutically acceptable salt, stereoisomer or tautomer thereof, G₄ is CR^{4a}. In some embodiments, R^{4a} is hydrogen, halo, —CN, —OR^{f}, NR^{g}R^{h}, C₁-C₆ alkyl, or C₆-C₁₄ aryl. In some embodiments, R^{4a} is hydrogen, halo, C₁-C₆ alkyl, or C₆-C₁₄ aryl. In some embodiments, R^{4a} is hydrogen, or halo. In some embodiments, R^{4a} is —H, —F, —Cl, —Br, —CH₃, —CH₂F, —CF₃, —CH₂OH, —CN, or —NH₂. In other embodiments, G₄ is N.

In some embodiments of the compound of formula (I), or a pharmaceutically acceptable salt, stereoisomer or tautomer thereof, m is 0, 1, or 2. In one variation, m is 0. In another variation, m is 1. In yet another variation, m is 2.

In some embodiments of the compound of formula (I), or a pharmaceutically acceptable salt, stereoisomer or tautomer thereof, n is 0, 1 or 2. In one variation, n is 0. In another variation, n is 1. In yet another variation, n is 2.

In some embodiments of the compound of formula (I), or a pharmaceutically acceptable salt, stereoisomer or tautomer thereof,

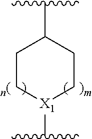

is a moiety selected from the group consisting of:

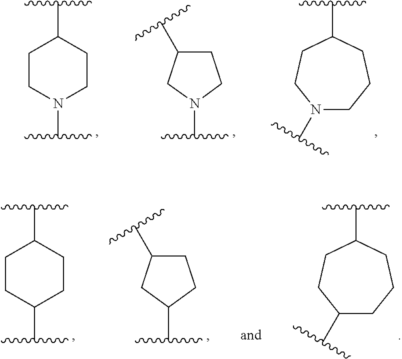 [SMILES: *C1CCN(*)CC1 ; *C1CCN(*)C1 ; *C1CCCN(*)CC1 ; *C1CCC(*)CC1 ; *C1CCC(*)C1 ; *C1CCCC(*)CC1]

In some embodiments of the compound of formula (I), or a pharmaceutically acceptable salt, stereoisomer or tautomer thereof,

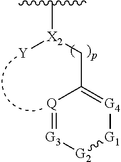

is a moiety selected from the group consisting of:

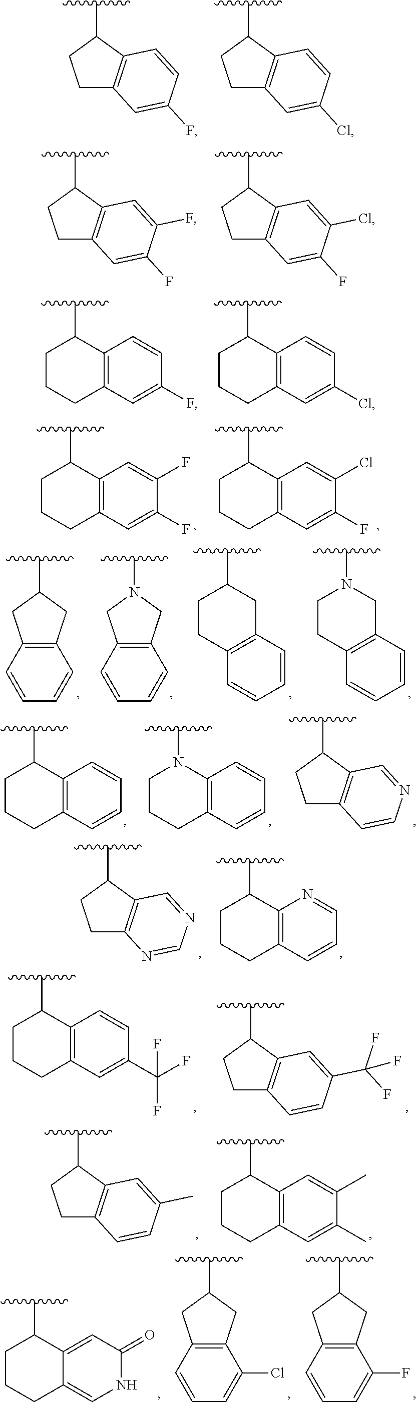

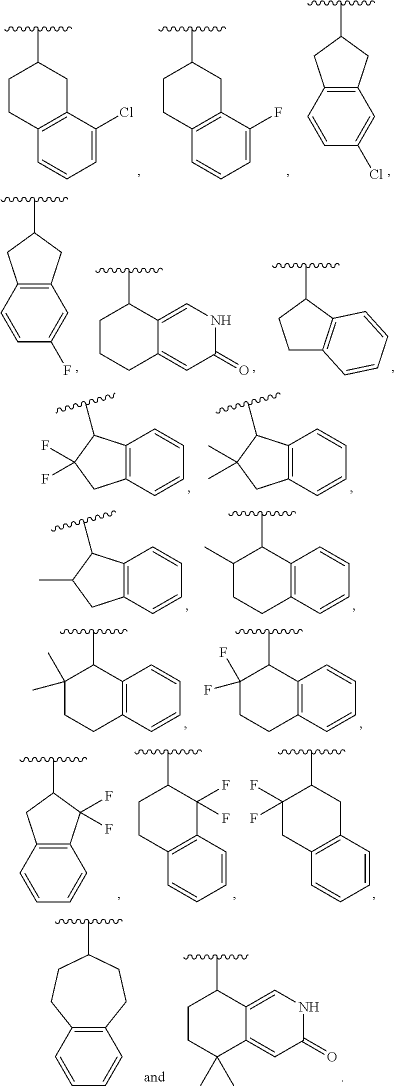

In other embodiments,

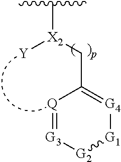

is a moiety selected from the group consisting of:

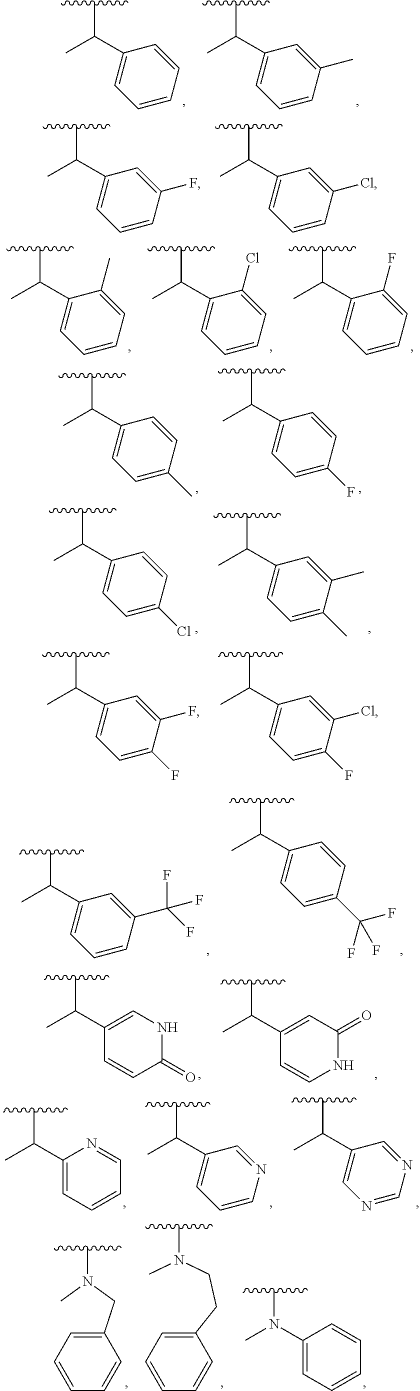

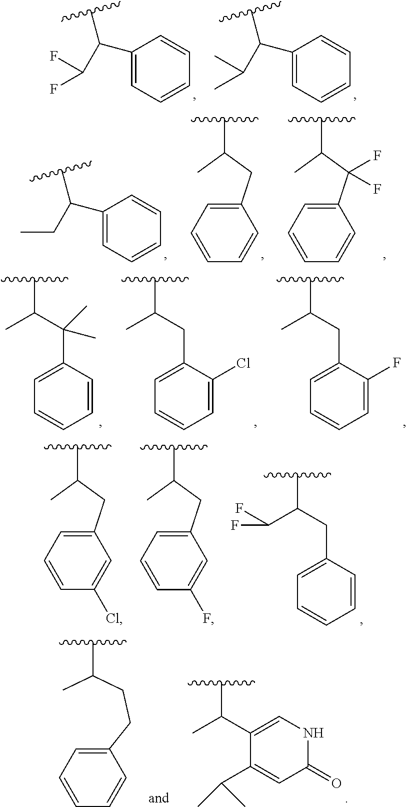

In some embodiments, provided herein are compounds described in Table A, or a pharmaceutically acceptable salt, stereoisomer or tautomer thereof, and uses thereof.

Representative compounds are listed in Table A.

| Compound | |
|---|---|
| Number | Structure |
| 1 | 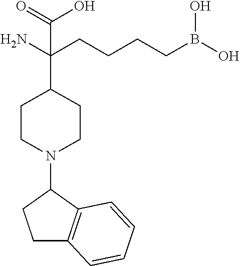 [SMILES: NC(CCCCB(O)O)(C(=O)O)C1CCN(C2CCc3ccccc32)CC1] |
| 2 | 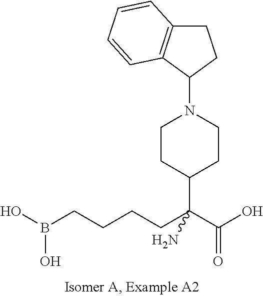 [SMILES: NC(CCCCB(O)O)(C(=O)O)C1CCN(C2CCc3ccccc32)CC1] |
| 3 | 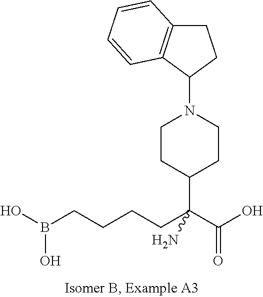 [SMILES: NC(CCCCB(O)O)(C(=O)O)C1CCN(C2CCc3ccccc32)CC1] |
| 4 | 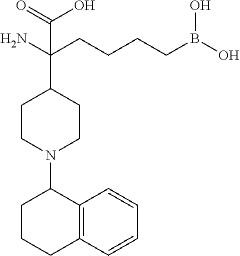 [SMILES: NC(CCCCB(O)O)(C(=O)O)C1CCN(C2CCCc3ccccc32)CC1] |
| 5 | 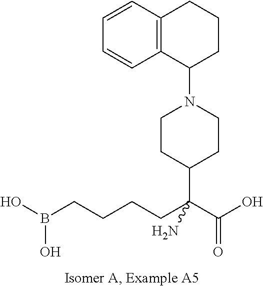 [SMILES: NC(CCCCB(O)O)(C(=O)O)C1CCN(C2CCCc3ccccc32)CC1] |
| 6 | 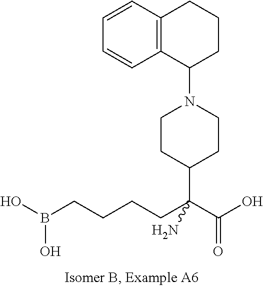 [SMILES: NC(CCCCB(O)O)(C(=O)O)C1CCN(C2CCCc3ccccc32)CC1] |
| 7 | 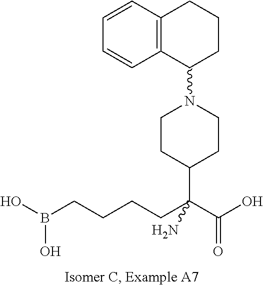 [SMILES: NC(CCCCB(O)O)(C(=O)O)C1CCN(C2CCCc3ccccc32)CC1] |
| 8 | 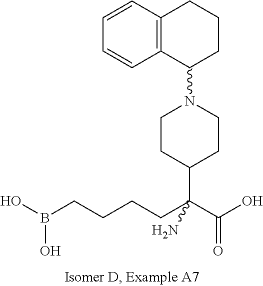 [SMILES: NC(CCCCB(O)O)(C(=O)O)C1CCN(C2CCCc3ccccc32)CC1] |
| 9 | 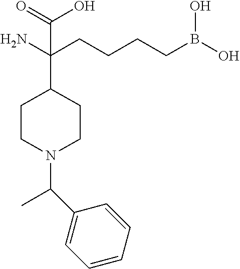 [SMILES: CC(c1ccccc1)N1CCC(C(N)(CCCCB(O)O)C(=O)O)CC1] |
| 10 | 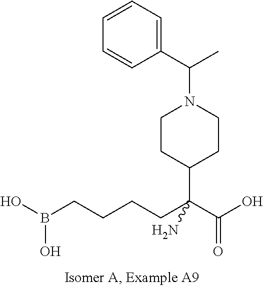 [SMILES: CC(c1ccccc1)N1CCC(C(N)(CCCCB(O)O)C(=O)O)CC1] |
| 11 | 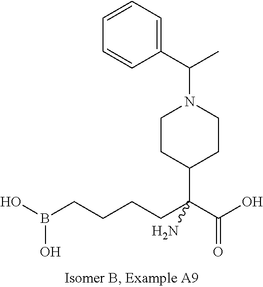 [SMILES: CC(c1ccccc1)N1CCC(C(N)(CCCCB(O)O)C(=O)O)CC1] |
| 12 | 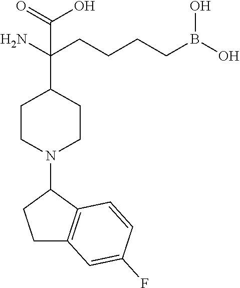 [SMILES: NC(CCCCB(O)O)(C(=O)O)C1CCN(C2CCc3cc(F)ccc32)CC1] |
| 13 | 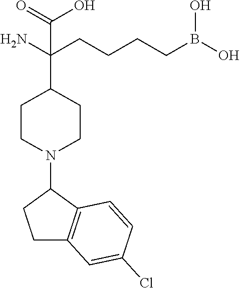 [SMILES: NC(CCCCB(O)O)(C(=O)O)C1CCN(C2CCc3cc(Cl)ccc32)CC1] |
| 14 | 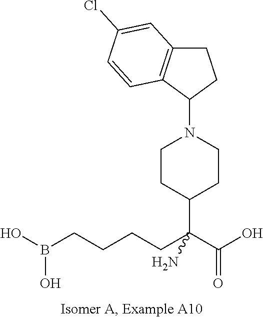 [SMILES: NC(CCCCB(O)O)(C(=O)O)C1CCN(C2CCc3cc(Cl)ccc32)CC1] |
| 15 | 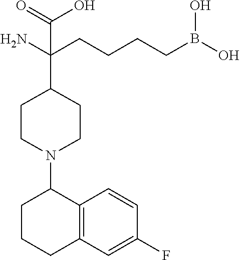 [SMILES: NC(CCCCB(O)O)(C(=O)O)C1CCN(C2CCCc3cc(F)ccc32)CC1] |
| 16 | 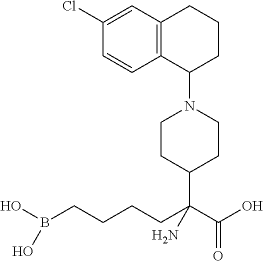 [SMILES: NC(CCCCB(O)O)(C(=O)O)C1CCN(C2CCCc3cc(Cl)ccc32)CC1] |
| 17 | 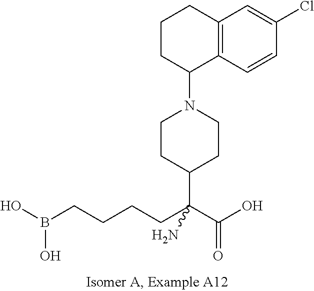 [SMILES: NC(CCCCB(O)O)(C(=O)O)C1CCN(C2CCCc3cc(Cl)ccc32)CC1] |
| 18 | 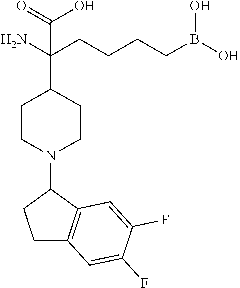 [SMILES: NC(CCCCB(O)O)(C(=O)O)C1CCN(C2CCc3cc(F)c(F)cc32)CC1] |
| 19 | 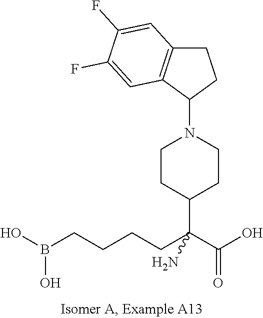 [SMILES: NC(CCCCB(O)O)(C(=O)O)C1CCN(C2CCc3cc(F)c(F)cc32)CC1] |
| 20 | 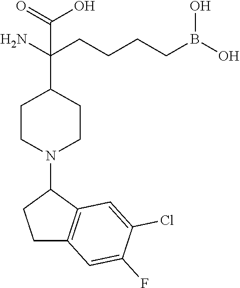 [SMILES: NC(CCCCB(O)O)(C(=O)O)C1CCN(C2CCc3cc(F)c(Cl)cc32)CC1] |
| 21 | 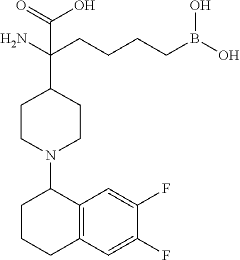 [SMILES: NC(CCCCB(O)O)(C(=O)O)C1CCN(C2CCCc3cc(F)c(F)cc32)CC1] |
| 22 | 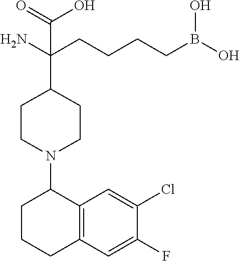 [SMILES: NC(CCCCB(O)O)(C(=O)O)C1CCN(C2CCCc3cc(F)c(Cl)cc32)CC1] |
| 23 | 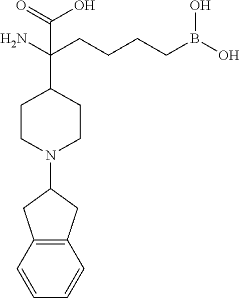 [SMILES: NC(CCCCB(O)O)(C(=O)O)C1CCN(C2Cc3ccccc3C2)CC1] |
| 24 | 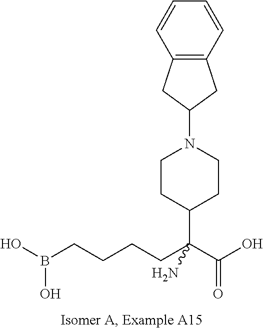 [SMILES: NC(CCCCB(O)O)(C(=O)O)C1CCN(C2Cc3ccccc3C2)CC1] |
| 25 | 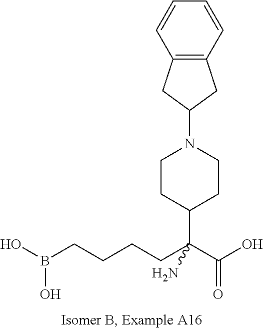 [SMILES: NC(CCCCB(O)O)(C(=O)O)C1CCN(C2Cc3ccccc3C2)CC1] |
| 26 | 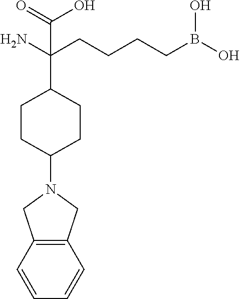 [SMILES: NC(CCCCB(O)O)(C(=O)O)C1CCC(N2Cc3ccccc3C2)CC1] |
| 27 | 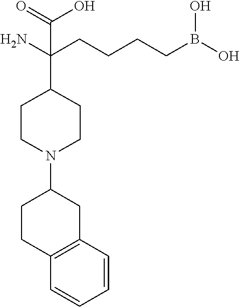 [SMILES: NC(CCCCB(O)O)(C(=O)O)C1CCN(C2CCc3ccccc3C2)CC1] |
| 28 | 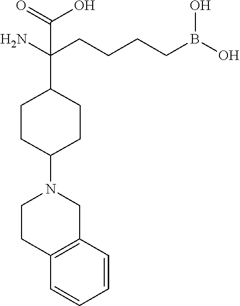 [SMILES: NC(CCCCB(O)O)(C(=O)O)C1CCC(N2CCc3ccccc3C2)CC1] |
| 29 | 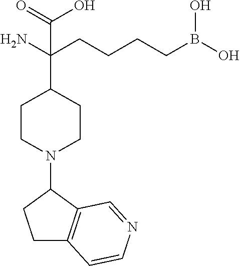 [SMILES: NC(CCCCB(O)O)(C(=O)O)C1CCN(C2CCc3ccncc32)CC1] |
| 30 | 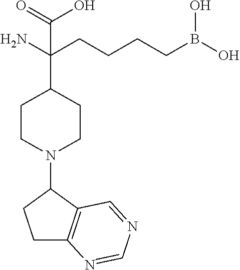 [SMILES: NC(CCCCB(O)O)(C(=O)O)C1CCN(C2CCc3ncncc32)CC1] |
| 31 | 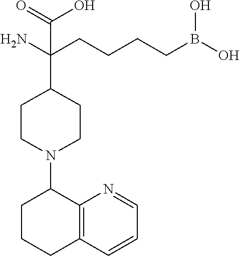 [SMILES: NC(CCCCB(O)O)(C(=O)O)C1CCN(C2CCCc3cccnc32)CC1] |
| 32 | 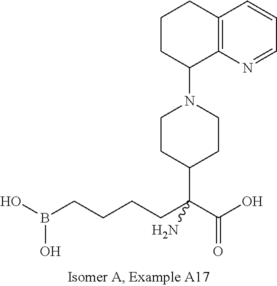 [SMILES: NC(CCCCB(O)O)(C(=O)O)C1CCN(C2CCCc3cccnc32)CC1] |
| 33 | 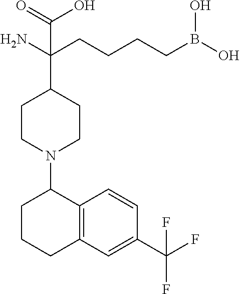 [SMILES: NC(CCCCB(O)O)(C(=O)O)C1CCN(C2CCCc3cc(C(F)(F)F)ccc32)CC1] |
| 34 | 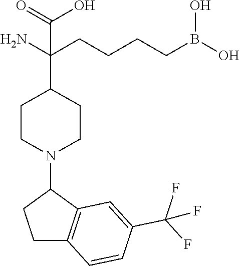 [SMILES: NC(CCCCB(O)O)(C(=O)O)C1CCN(C2CCc3ccc(C(F)(F)F)cc32)CC1] |
| 35 | 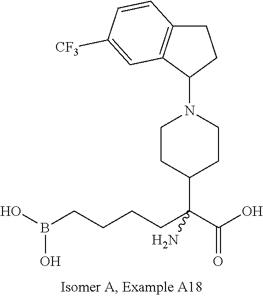 [SMILES: NC(CCCCB(O)O)(C(=O)O)C1CCN(C2CCc3ccc(C(F)(F)F)cc32)CC1] |
| 36 | 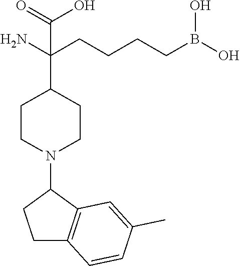 [SMILES: Cc1ccc2c(c1)C(N1CCC(C(N)(CCCCB(O)O)C(=O)O)CC1)CC2] |
| 37 | 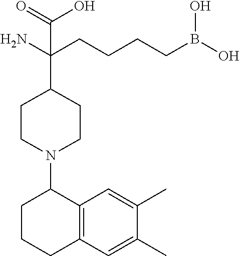 [SMILES: Cc1cc2c(cc1C)C(N1CCC(C(N)(CCCCB(O)O)C(=O)O)CC1)CCC2] |
| 38 | 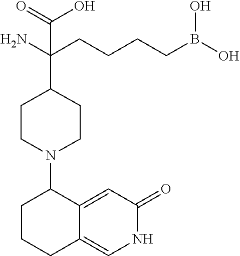 [SMILES: NC(CCCCB(O)O)(C(=O)O)C1CCN(C2CCCc3c[nH]c(=O)cc32)CC1] |
| 39 | 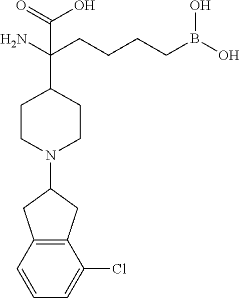 [SMILES: NC(CCCCB(O)O)(C(=O)O)C1CCN(C2Cc3cccc(Cl)c3C2)CC1] |
| 40 | 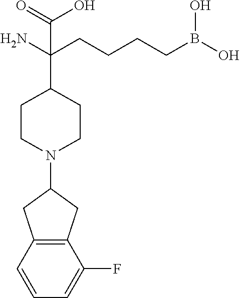 [SMILES: NC(CCCCB(O)O)(C(=O)O)C1CCN(C2Cc3cccc(F)c3C2)CC1] |
| 41 | 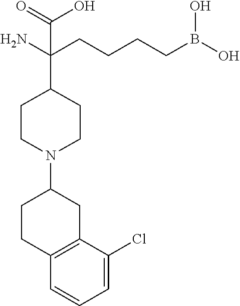 [SMILES: NC(CCCCB(O)O)(C(=O)O)C1CCN(C2CCc3cccc(Cl)c3C2)CC1] |
| 42 | 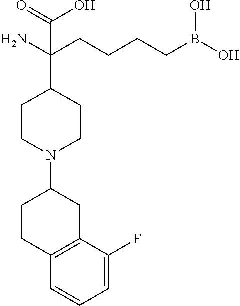 [SMILES: NC(CCCCB(O)O)(C(=O)O)C1CCN(C2CCc3cccc(F)c3C2)CC1] |
| 43 | 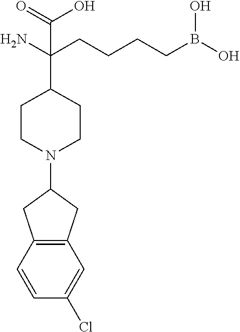 [SMILES: NC(CCCCB(O)O)(C(=O)O)C1CCN(C2Cc3ccc(Cl)cc3C2)CC1] |
| 44 | 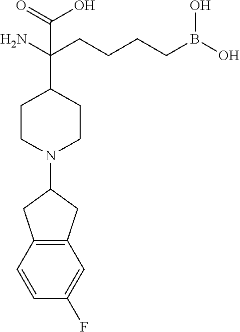 [SMILES: NC(CCCCB(O)O)(C(=O)O)C1CCN(C2Cc3ccc(F)cc3C2)CC1] |
| 45 | 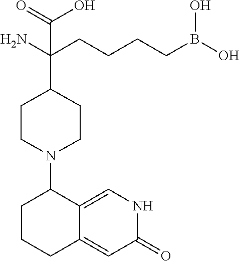 [SMILES: NC(CCCCB(O)O)(C(=O)O)C1CCN(C2CCCc3cc(=O)[nH]cc32)CC1] |
| 46 | 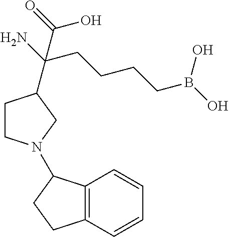 [SMILES: NC(CCCCB(O)O)(C(=O)O)C1CCN(C2CCc3ccccc32)C1] |
| 47 | 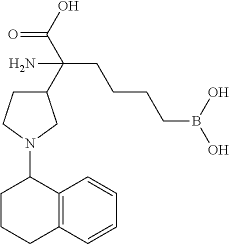 [SMILES: NC(CCCCB(O)O)(C(=O)O)C1CCN(C2CCCc3ccccc32)C1] |
| 48 | 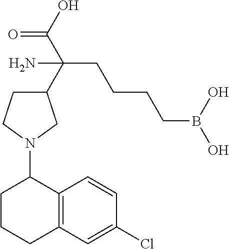 [SMILES: NC(CCCCB(O)O)(C(=O)O)C1CCN(C2CCCc3cc(Cl)ccc32)C1] |
| 49 | 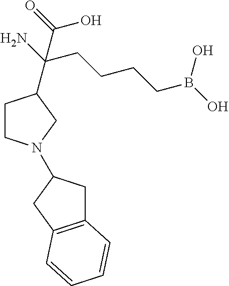 [SMILES: NC(CCCCB(O)O)(C(=O)O)C1CCN(C2Cc3ccccc3C2)C1] |
| 50 | 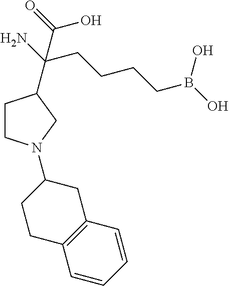 [SMILES: NC(CCCCB(O)O)(C(=O)O)C1CCN(C2CCc3ccccc3C2)C1] |
| 51 | 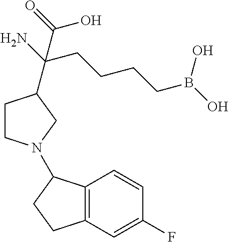 [SMILES: NC(CCCCB(O)O)(C(=O)O)C1CCN(C2CCc3cc(F)ccc32)C1] |
| 52 | 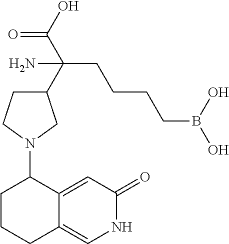 [SMILES: NC(CCCCB(O)O)(C(=O)O)C1CCN(C2CCCc3c[nH]c(=O)cc32)C1] |
| 53 | 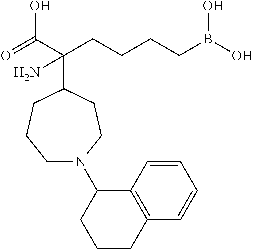 [SMILES: NC(CCCCB(O)O)(C(=O)O)C1CCCN(C2CCCc3ccccc32)CC1] |
| 54 | 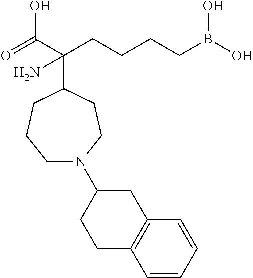 [SMILES: NC(CCCCB(O)O)(C(=O)O)C1CCCN(C2CCc3ccccc3C2)CC1] |
| 55 | 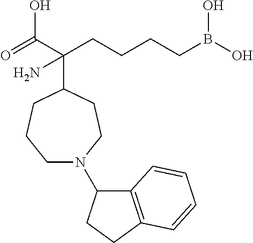 [SMILES: NC(CCCCB(O)O)(C(=O)O)C1CCCN(C2CCc3ccccc32)CC1] |
| 56 | 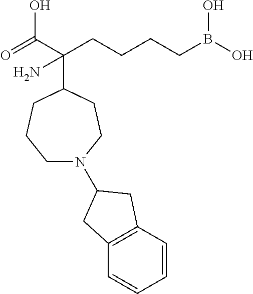 [SMILES: NC(CCCCB(O)O)(C(=O)O)C1CCCN(C2Cc3ccccc3C2)CC1] |
| 57 | 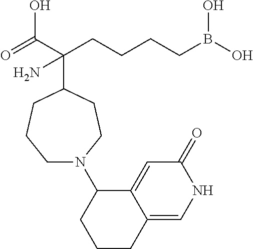 [SMILES: NC(CCCCB(O)O)(C(=O)O)C1CCCN(C2CCCc3c[nH]c(=O)cc32)CC1] |
| 58 | 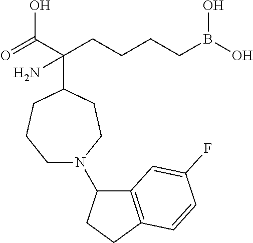 [SMILES: NC(CCCCB(O)O)(C(=O)O)C1CCCN(C2CCc3ccc(F)cc32)CC1] |
| 59 | 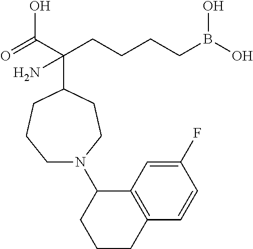 [SMILES: NC(CCCCB(O)O)(C(=O)O)C1CCCN(C2CCCc3ccc(F)cc32)CC1] |
| 60 | 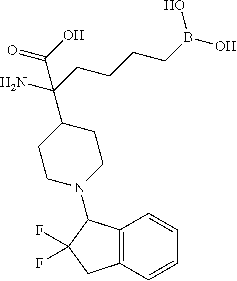 [SMILES: NC(CCCCB(O)O)(C(=O)O)C1CCN(C2c3ccccc3CC2(F)F)CC1] |
| 61 | 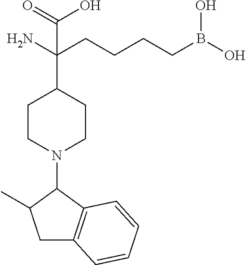 [SMILES: CC1Cc2ccccc2C1N1CCC(C(N)(CCCCB(O)O)C(=O)O)CC1] |
| 62 | 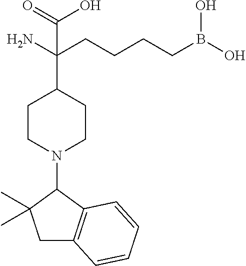 [SMILES: CC1(C)Cc2ccccc2C1N1CCC(C(N)(CCCCB(O)O)C(=O)O)CC1] |
| 63 | 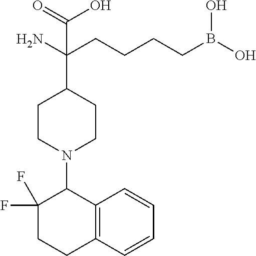 [SMILES: NC(CCCCB(O)O)(C(=O)O)C1CCN(C2c3ccccc3CCC2(F)F)CC1] |
| 64 | 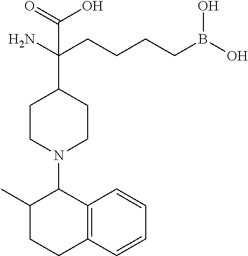 [SMILES: CC1CCc2ccccc2C1N1CCC(C(N)(CCCCB(O)O)C(=O)O)CC1] |
| 65 | 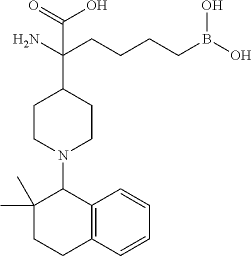 [SMILES: CC1(C)CCc2ccccc2C1N1CCC(C(N)(CCCCB(O)O)C(=O)O)CC1] |
| 66 | 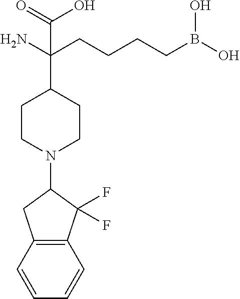 [SMILES: NC(CCCCB(O)O)(C(=O)O)C1CCN(C2Cc3ccccc3C2(F)F)CC1] |
| 67 | 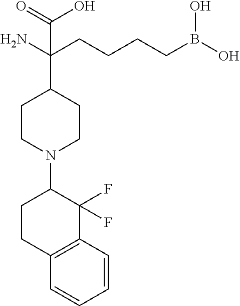 [SMILES: NC(CCCCB(O)O)(C(=O)O)C1CCN(C2CCc3ccccc3C2(F)F)CC1] |
| 68 | 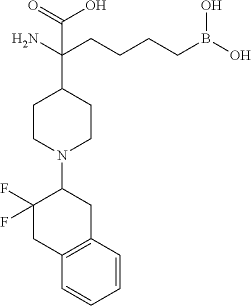 [SMILES: NC(CCCCB(O)O)(C(=O)O)C1CCN(C2Cc3ccccc3CC2(F)F)CC1] |
| 69 | 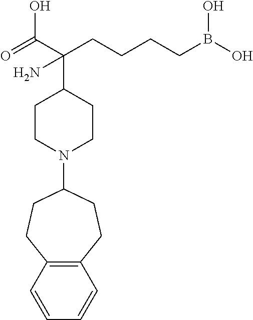 [SMILES: NC(CCCCB(O)O)(C(=O)O)C1CCN(C2CCc3ccccc3CC2)CC1] |
| 70 | 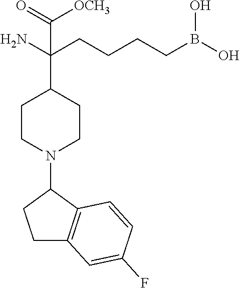 [SMILES: COC(=O)C(N)(CCCCB(O)O)C1CCN(C2CCc3cc(F)ccc32)CC1] |
| 71 | 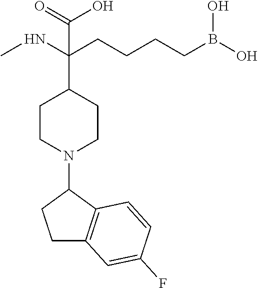 [SMILES: CNC(CCCCB(O)O)(C(=O)O)C1CCN(C2CCc3cc(F)ccc32)CC1] |
| 72 | 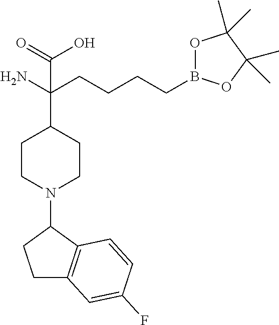 [SMILES: CC1(C)OB(CCCCC(N)(C(=O)O)C2CCN(C3CCc4cc(F)ccc43)CC2)OC1(C)C] |
| 73 | 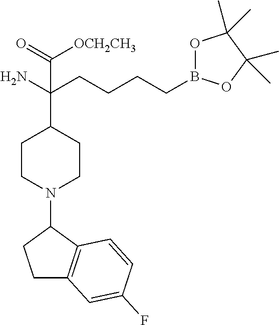 [SMILES: CCOC(=O)C(N)(CCCCB1OC(C)(C)C(C)(C)O1)C1CCN(C2CCc3cc(F)ccc32)CC1] |
| 74 | 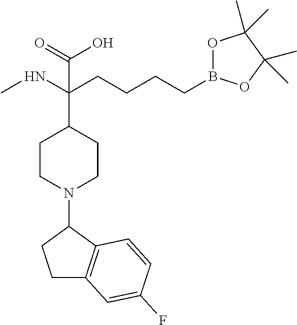 |
| 75 | 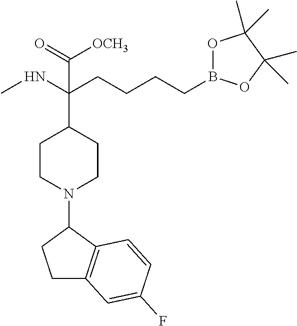 |
| 76 | 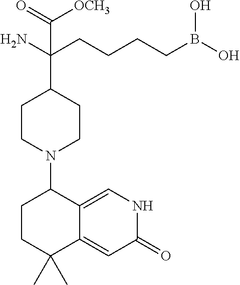 [SMILES: COC(=O)C(N)(CCCCB(O)O)C1CCN(C2CCC(C)(C)c3cc(=O)[nH]cc32)CC1] |
| 77 | 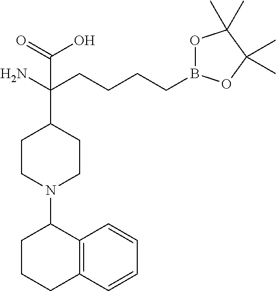 [SMILES: CC1(C)OB(CCCCC(N)(C(=O)O)C2CCN(C3CCCc4ccccc43)CC2)OC1(C)C] |
| 78 | 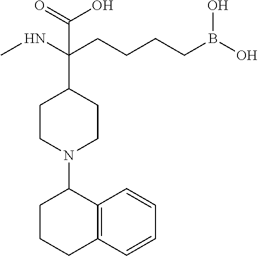 [SMILES: CNC(CCCCB(O)O)(C(=O)O)C1CCN(C2CCCc3ccccc32)CC1] |
| 79 | 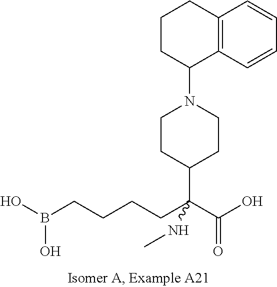 [SMILES: CNC(CCCCB(O)O)(C(=O)O)C1CCN(C2CCCc3ccccc32)CC1] |
| 80 | 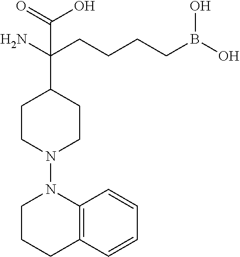 [SMILES: NC(CCCCB(O)O)(C(=O)O)C1CCN(N2CCCc3ccccc32)CC1] |
| 81 | 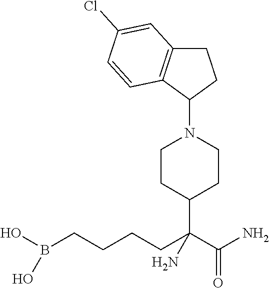 [SMILES: NC(=O)C(N)(CCCCB(O)O)C1CCN(C2CCc3cc(Cl)ccc32)CC1] |
| 82 | 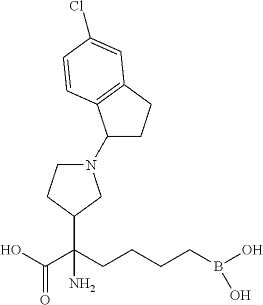 [SMILES: NC(CCCCB(O)O)(C(=O)O)C1CCN(C2CCc3cc(Cl)ccc32)C1] |
| 83 | 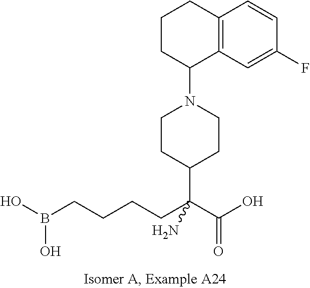 [SMILES: NC(CCCCB(O)O)(C(=O)O)C1CCN(C2CCCc3ccc(F)cc32)CC1] |
| 84 | 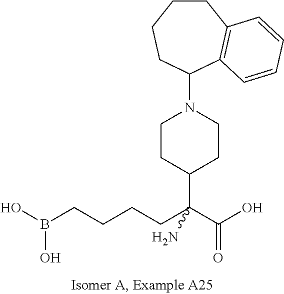 [SMILES: NC(CCCCB(O)O)(C(=O)O)C1CCN(C2CCCCc3ccccc32)CC1] |
| 85 | 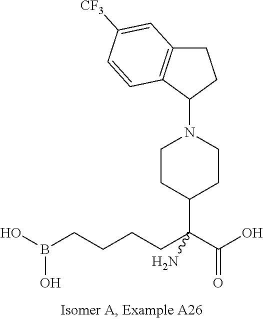 [SMILES: NC(CCCCB(O)O)(C(=O)O)C1CCN(C2CCc3cc(C(F)(F)F)ccc32)CC1] |
| 86 | 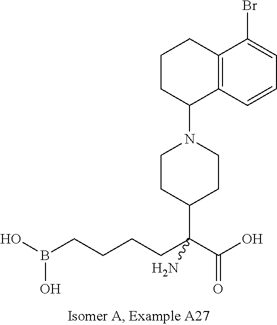 [SMILES: NC(CCCCB(O)O)(C(=O)O)C1CCN(C2CCCc3c(Br)cccc32)CC1] |
| 87 | 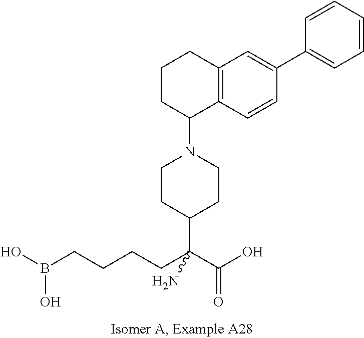 [SMILES: NC(CCCCB(O)O)(C(=O)O)C1CCN(C2CCCc3cc(-c4ccccc4)ccc32)CC1] |
| 88 | 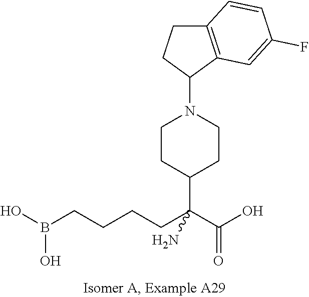 [SMILES: NC(CCCCB(O)O)(C(=O)O)C1CCN(C2CCc3ccc(F)cc32)CC1] |
| 89 | 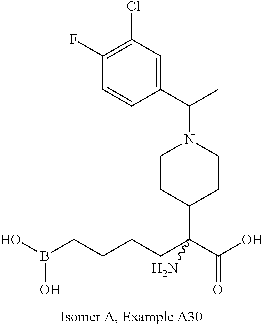 [SMILES: CC(c1ccc(F)c(Cl)c1)N1CCC(C(N)(CCCCB(O)O)C(=O)O)CC1] |
| 90 | 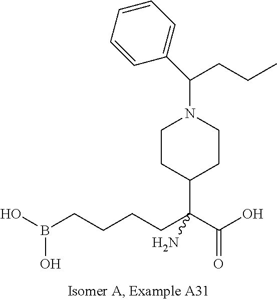 [SMILES: CCCC(c1ccccc1)N1CCC(C(N)(CCCCB(O)O)C(=O)O)CC1] |
| 91 | 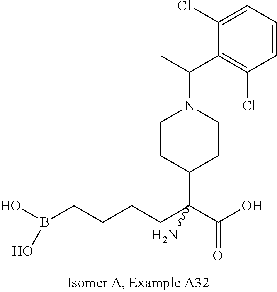 [SMILES: CC(c1c(Cl)cccc1Cl)N1CCC(C(N)(CCCCB(O)O)C(=O)O)CC1] |
| 92 | 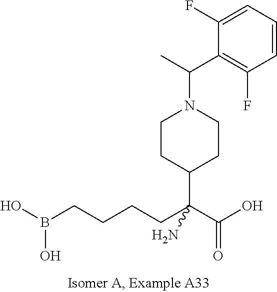 [SMILES: CC(c1c(F)cccc1F)N1CCC(C(N)(CCCCB(O)O)C(=O)O)CC1] |

In some embodiments, provided herein is a compound selected from the group consisting of
2-amino-6-borono-2-(1-(2,3-dihydro-1H-inden-1-yl)piperidin-4-yl)hexanoic acid;
2-amino-6-borono-2-(1-(1,2,3,4-tetrahydronaphthalen-1-yl)piperidin-4-yl)hexanoic acid;
2-amino-6-borono-2-(1-(1-phenylethyl)piperidin-4-yl)hexanoic acid;
2-amino-6-borono-2-(1-(5-fluoro-2,3-dihydro-1H-inden-1-yl)piperidin-4-yl)hexanoic acid;
2-amino-6-borono-2-(1-(5-chloro-2,3-dihydro-1H-inden-1-yl)piperidin-4-yl)hexanoic acid;
2-amino-6-borono-2-(1-(6-fluoro-1,2,3,4-tetrahydronaphthalen-1-yl)piperidin-4-yl)hexanoic acid;
2-amino-6-borono-2-(1-(6-chloro-1,2,3,4-tetrahydronaphthalen-1-yl)piperidin-4-yl)hexanoic acid;
2-amino-6-borono-2-(1-(5,6-difluoro-2,3-dihydro-1H-inden-1-yl)piperidin-4-yl)hexanoic acid;
2-amino-6-borono-2-(1-(6-chloro-5-fluoro-2,3-dihydro-1H-inden-1-yl)piperidin-4-yl)hexanoic acid;
2-amino-6-borono-2-(1-(6,7-difluoro-1,2,3,4-tetrahydronaphthalen-1-yl)piperidin-4-yl)hexanoic acid;
2-amino-6-borono-2-(1-(7-chloro-6-fluoro-1,2,3,4-tetrahydronaphthalen-1-yl)piperidin-4-yl)hexanoic acid;
2-amino-6-borono-2-(1-(2,3-dihydro-1H-inden-2-yl)piperidin-4-yl)hexanoic acid;
2-amino-6-borono-2-(4-(isoindolin-2-yl)cyclohexyl)hexanoic acid;
2-amino-6-borono-2-(1-(1,2,3,4-tetrahydronaphthalen-2-yl)piperidin-4-yl)hexanoic acid;
2-amino-6-borono-2-(4-(3,4-dihydroisoquinolin-2(1H)-yl)cyclohexyl)hexanoic acid;
2-amino-6-borono-2-(1-(6,7-dihydro-5H-cyclopenta[c]pyridin-7-yl)piperidin-4-yl)hexanoic acid;
2-amino-6-borono-2-(1-(6,7-dihydro-5H-cyclopenta[d]pyrimidin-5-yl)piperidin-4-yl)hexanoic acid;
2-amino-6-borono-2-(1-(5,6,7,8-tetrahydroquinolin-8-yl)piperidin-4-yl)hexanoic acid;
2-amino-6-borono-2-(1-(6-(trifluoromethyl)-1,2,3,4-tetrahydronaphthalen-1-yl)piperidin-4-yl)hexanoic acid;
2-amino-6-borono-2-(1-(6-(trifluoromethyl)-2,3-dihydro-1H-inden-1-yl)piperidin-4-yl)hexanoic acid;
2-amino-6-borono-2-(1-(6-methyl-2,3-dihydro-1H-inden-1-yl)piperidin-4-yl)hexanoic acid;
2-amino-6-borono-2-(1-(6,7-dimethyl-1,2,3,4-tetrahydronaphthalen-1-yl)piperidin-4-yl)hexanoic acid;
2-amino-6-borono-2-(1-(3-oxo-2,3,5,6,7,8-hexahydroisoquinolin-5-yl)piperidin-4-yl)hexanoic acid;
2-amino-6-borono-2-(1-(4-chloro-2,3-dihydro-1H-inden-2-yl)piperidin-4-yl)hexanoic acid;
2-amino-6-borono-2-(1-(4-fluoro-2,3-dihydro-1H-inden-2-yl)piperidin-4-yl)hexanoic acid;
2-amino-6-borono-2-(1-(8-chloro-1,2,3,4-tetrahydronaphthalen-2-yl)piperidin-4-yl)hexanoic acid;
2-amino-6-borono-2-(1-(8-fluoro-1,2,3,4-tetrahydronaphthalen-2-yl)piperidin-4-yl)hexanoic acid;
2-amino-6-borono-2-(1-(5-chloro-2,3-dihydro-1H-inden-2-yl)piperidin-4-yl)hexanoic acid;
2-amino-6-borono-2-(1-(5-fluoro-2,3-dihydro-1H-inden-2-yl)piperidin-4-yl)hexanoic acid;
2-amino-6-borono-2-(1-(3-oxo-2,3,5,6,7,8-hexahydroisoquinolin-8-yl)piperidin-4-yl)hexanoic acid;
2-amino-6-borono-2-(1-(2,3-dihydro-1H-inden-1-yl)pyrrolidin-3-yl)hexanoic acid;
2-amino-6-borono-2-(1-(1,2,3,4-tetrahydronaphthalen-1-yl)pyrrolidin-3-yl)hexanoic acid;
2-amino-6-borono-2-(1-(6-chloro-1,2,3,4-tetrahydronaphthalen-1-yl)pyrrolidin-3-yl)hexanoic acid;
2-amino-6-borono-2-(1-(2,3-dihydro-1H-inden-2-yl)pyrrolidin-3-yl)hexanoic acid;
2-amino-6-borono-2-(1-(1,2,3,4-tetrahydronaphthalen-2-yl)pyrrolidin-3-yl)hexanoic acid;
2-amino-6-borono-2-(1-(5-fluoro-2,3-dihydro-1H-inden-1-yl)pyrrolidin-3-yl)hexanoic acid;
2-amino-6-borono-2-(1-(3-oxo-2,3,5,6,7,8-hexahydroisoquinolin-5-yl)pyrrolidin-3-yl)hexanoic acid;
2-amino-6-borono-2-(1-(1,2,3,4-tetrahydronaphthalen-1-yl)azepan-4-yl)hexanoic acid;
2-amino-6-borono-2-(1-(1,2,3,4-tetrahydronaphthalen-2-yl)azepan-4-yl)hexanoic acid;
2-amino-6-borono-2-(1-(2,3-dihydro-1H-inden-1-yl)azepan-4-yl)hexanoic acid;
2-amino-6-borono-2-(1-(2,3-dihydro-1H-inden-2-yl)azepan-4-yl)hexanoic acid;
2-amino-6-borono-2-(1-(3-oxo-2,3,5,6,7,8-hexahydroisoquinolin-5-yl)azepan-4-yl)hexanoic acid;
2-amino-6-borono-2-(1-(6-fluoro-2,3-dihydro-1H-inden-1-yl)azepan-4-yl)hexanoic acid;
2-amino-6-borono-2-(1-(7-fluoro-1,2,3,4-tetrahydronaphthalen-1-yl)azepan-4-yl)hexanoic acid;
2-amino-6-borono-2-(1-(2,2-difluoro-2,3-dihydro-1H-inden-1-yl)piperidin-4-yl)hexanoic acid;
2-amino-6-borono-2-(1-(2-methyl-2,3-dihydro-1H-inden-1-yl)piperidin-4-yl)hexanoic acid;
2-amino-6-borono-2-(1-(2,2-dimethyl-2,3-dihydro-1H-inden-1-yl)piperidin-4-yl)hexanoic acid;
2-amino-6-borono-2-(1-(2,2-difluoro-1,2,3,4-tetrahydronaphthalen-1-yl)piperidin-4-yl)hexanoic acid;
2-amino-6-borono-2-(1-(2-methyl-1,2,3,4-tetrahydronaphthalen-1-yl)piperidin-4-yl)hexanoic acid;
2-amino-6-borono-2-(1-(2,2-dimethyl-1,2,3,4-tetrahydronaphthalen-1-yl)piperidin-4-yl)hexanoic acid;
2-amino-6-borono-2-(1-(1,1-difluoro-2,3-dihydro-1H-inden-2-yl)piperidin-4-yl)hexanoic acid;
2-amino-6-borono-2-(1-(1,1-difluoro-1,2,3,4-tetrahydronaphthalen-2-yl)piperidin-4-yl)hexanoic acid;
2-amino-6-borono-2-(1-(3,3-difluoro-1,2,3,4-tetrahydronaphthalen-2-yl)piperidin-4-yl)hexanoic acid;
2-amino-6-borono-2-(1-(6,7,8,9-tetrahydro-5H-benzo[7]annulen-7-yl)piperidin-4-yl)hexanoic acid;
(5-amino-5-(1-(5-fluoro-2,3-dihydro-1H-inden-1-yl)piperidin-4-yl)-6-methoxy-6-oxohexyl)boronic acid;
6-borono-2-(1-(5-fluoro-2,3-dihydro-1H-inden-1-yl)piperidin-4-yl)-2-(methylamino)hexanoic acid;
2-amino-2-(1-(5-fluoro-2,3-dihydro-1H-inden-1-yl)piperidin-4-yl)-6-(4,4,5,5-tetramethyl-1,3,2-dioxaborolan-2-yl)hexanoic acid;
ethyl 2-amino-2-(1-(5-fluoro-2,3-dihydro-1H-inden-1-yl)piperidin-4-yl)-6-(4,4,5,5-tetramethyl-1,3,2-dioxaborolan-2-yl)hexanoate;
2-(1-(5-fluoro-2,3-dihydro-1H-inden-1-yl)piperidin-4-yl)-2-(methylamino)-6-(4,4,5,5-tetramethyl-1,3,2-dioxaborolan-2-yl)hexanoic acid;
methyl 2-(1-(5-fluoro-2,3-dihydro-1H-inden-1-yl)piperidin-4-yl)-2-(methylamino)-6-(4,4,5,5-tetramethyl-1,3,2-dioxaborolan-2-yl)hexanoate;
2-amino-6-borono-2-(1-(5,5-dimethyl-3-oxo-2,3,5,6,7,8-hexahydroisoquinolin-8-yl)piperidin-4-yl)hexanoic acid;
2-amino-2-(1-(1,2,3,4-tetrahydronaphthalen-1-yl)piperidin-4-yl)-6-(4,4,5,5-tetramethyl-1,3,2-dioxaborolan-2-yl)hexanoic acid;
6-borono-2-(methylamino)-2-(1-(1,2,3,4-tetrahydronaphthalen-1-yl)piperidin-4-yl)hexanoic acid;
2-amino-6-borono-2-(1-(3,4-dihydroquinolin-1(2H)-yl)piperidin-4-yl)hexanoic acid;
(5,6-diamino-5-(1-(5-chloro-2,3-dihydro-1H-inden-1-yl)piperidin-4-yl)-6-oxohexyl)boronic acid;
2-amino-6-borono-2-(1-(5-chloro-2,3-dihydro-1H-inden-1-yl)pyrrolidin-3-yl)hexanoic acid;
2-amino-6-borono-2-(1-(7-fluoro-1,2,3,4-tetrahydronaphthalen-1-yl)piperidin-4-yl)hexanoic acid;
2-amino-6-borono-2-(1-(6,7,8,9-tetrahydro-5H-benzo[7]annulen-5-yl)piperidin-4-yl)hexanoic acid;
2-amino-6-borono-2-(1-(5-(trifluoromethyl)-2,3-dihydro-1H-inden-1-yl)piperidin-4-yl)hexanoic acid;
2-amino-6-borono-2-(1-(5-bromo-1,2,3,4-tetrahydronaphthalen-1-yl)piperidin-4-yl)hexanoic acid;
2-amino-6-borono-2-(1-(6-phenyl-1,2,3,4-tetrahydronaphthalen-1-yl)piperidin-4-yl)hexanoic acid;
2-amino-6-borono-2-(1-(6-fluoro-2,3-dihydro-1H-inden-1-yl)piperidin-4-yl)hexanoic acid;
2-amino-6-borono-2-(1-(1-(3-chloro-4-fluorophenyl)ethyl)piperidin-4-yl)hexanoic acid;
2-amino-6-borono-2-(1-(1-phenylbutyl)piperidin-4-yl)hexanoic acid;
2-amino-6-borono-2-(1-(1-(2,6-dichlorophenyl)ethyl)piperidin-4-yl)hexanoic acid; and
2-amino-6-borono-2-(1-(1-(2,6-difluorophenyl)ethyl)piperidin-4-yl)hexanoic acid,
   or a pharmaceutically acceptable salt thereof. Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

Representative stereoisomers include, but are not limited to:

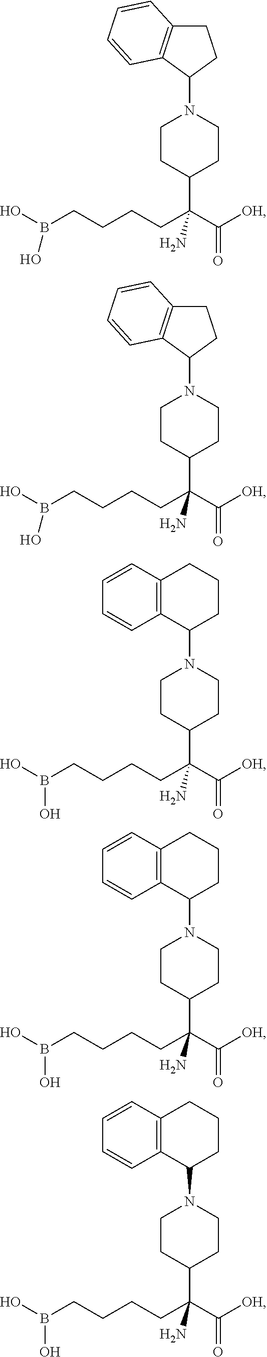

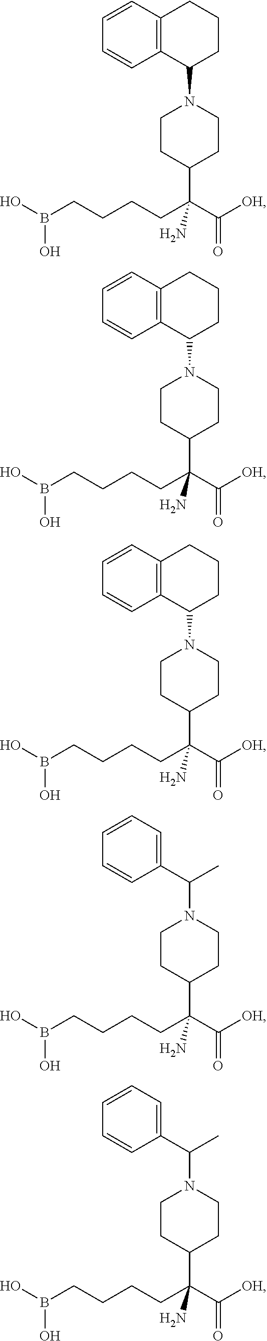

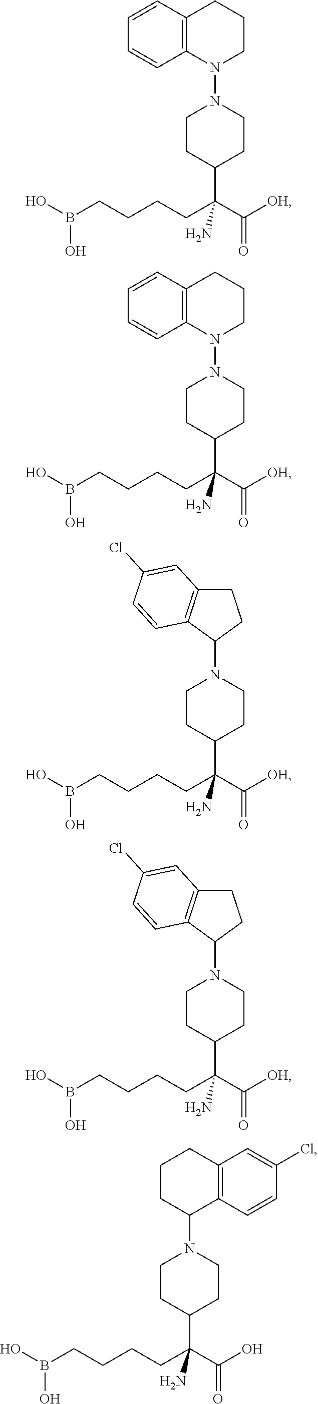

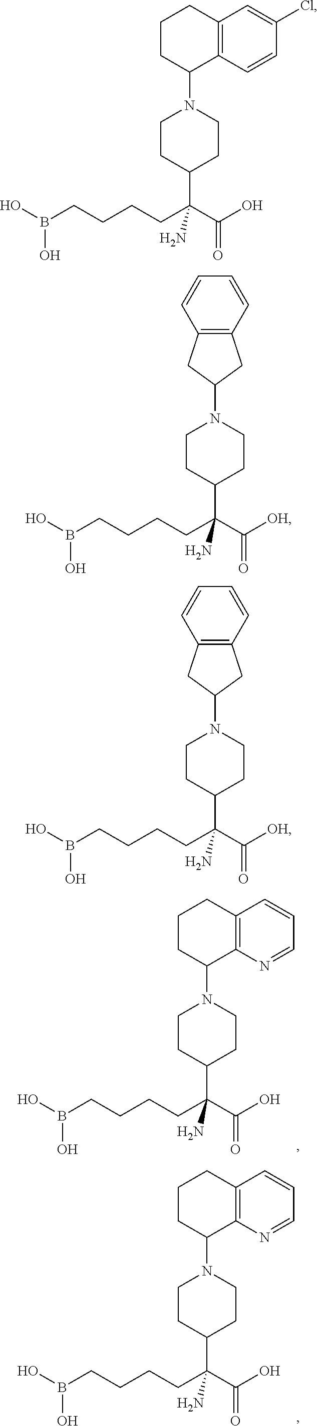

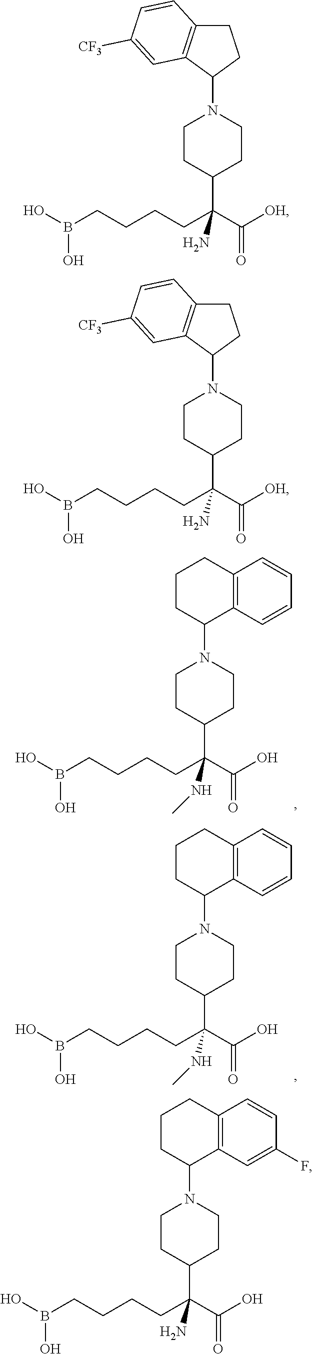

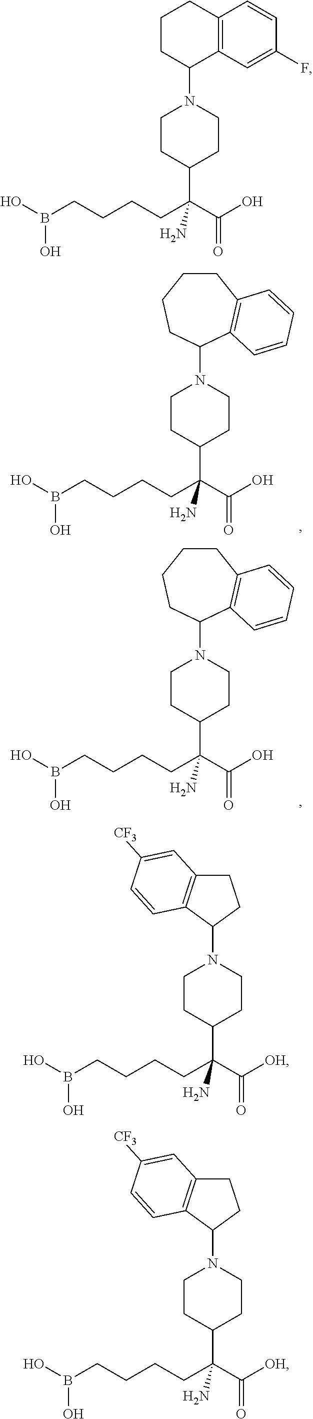

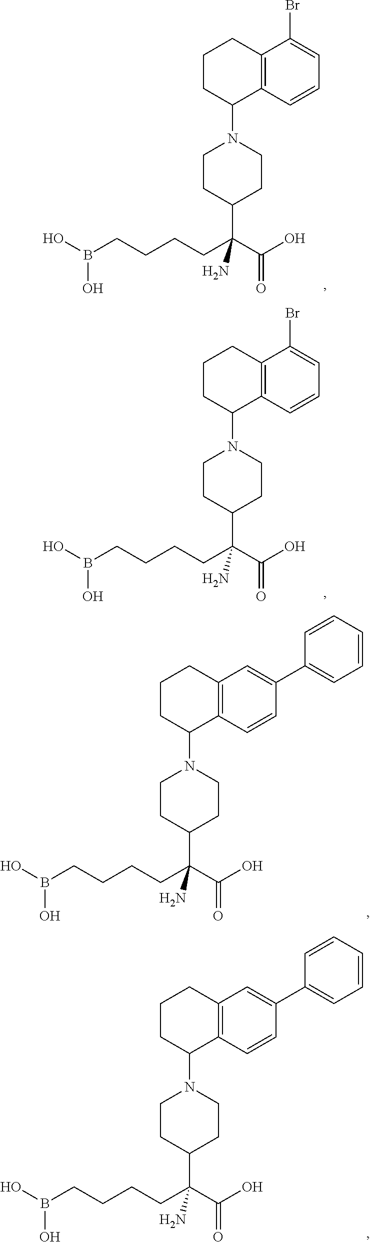

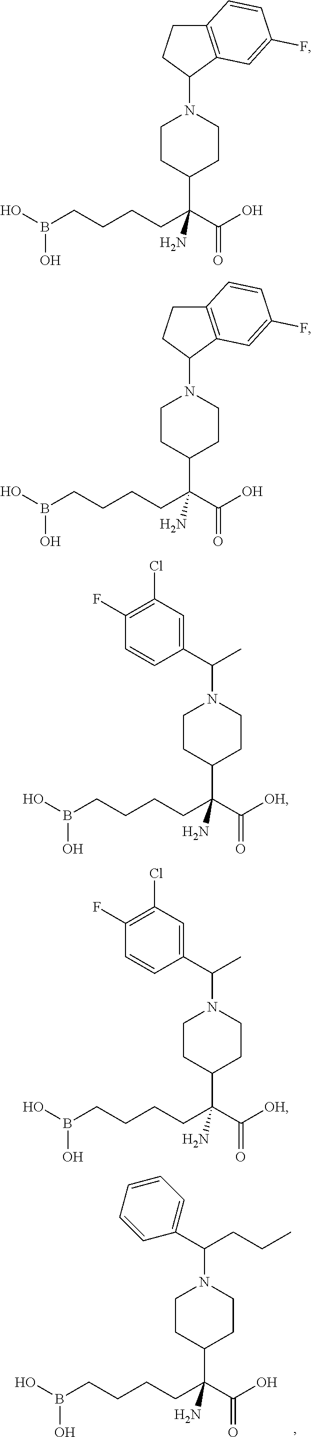

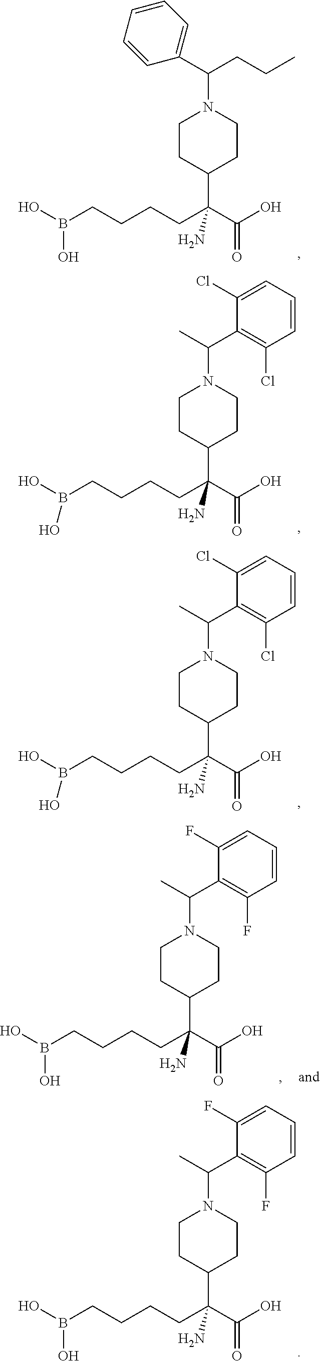

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

As detailed herein, including in the synthetic examples, certain compounds bear one or more stereocenters. If a compound is present as a single stereoisomer (such as when separated from a corresponding alternate stereoisomer or prepared in a stereospecific manner), the one or more stereocenters in the compound are indicated by a wavy bond ( [symbol] ) to a substituent. The presence of a wavy bond in the specific compound species of the compounds presented herein indicates a single stereoisomer (for example, a single enantiomer). Thus, it will be appreciated that a compound in Table A may exist as a single stereoisomer or as a mixture of stereoisomers. For example, compound 2 from Table A represents a single stereoisomer (see synthetic Example A2), and compound 3 from Table A represents a different single stereoisomer (see synthetic Example A3). Thus, although Table A illustrates compounds 2 and 3 by the same chemical structure that bears a wavy bond, it is appreciated that compound 2, as illustrated in Table A, corresponds to the stereoisomer (Isomer A) prepared according to synthetic Example A2 and compound 3, as illustrated in Table A, corresponds to the stereoisomer (Isomer B) prepared according to synthetic Example A3. It is appreciated that the data tables presented herein, for example, Tables 1, 2, and 3, will provide the data associated with a particular stereoisomer by using compound-number designations and, where applicable, synthetic-example designations. For example, reference to “Compound 2 (Isomer A, Example A2)” in the biological-data Tables 1-3 will indicate that the data were obtained with the isomer prepared according to synthetic Example A2 (Isomer A); reference to “Compound 3 (Isomer B, Example A3)” in the biological-data Tables 1-3 will indicate that the data were obtained with the isomer prepared according to synthetic Example A3 (Isomer B).

In some embodiments, a pair of stereoisomers (including, for example, a pair of enantiomers or diastereomers) may be separated by any suitable method, including, but not limited to, chiral HPLC. When a pair of stereoisomers is separated by HPLC, it is to be appreciated that the resultant individual stereoisomers will be assigned sequential labels (e.g., A, B, etc.), the order of which implies the order in which the isomers eluted from the HPLC column. For example, when a pair of stereoisomers is separated by HPLC, it is to be appreciated that the first-eluting isomer is labeled “Isomer A,” and the second-eluting isomer is labeled “Isomer B.” The absolute stereochemistry for “Isomer A” and “Isomer B” may be obtained by known methods. In other embodiments, more than one stereocenter is present in a molecule, such as when a first compound containing a first stereocenter is separated into Isomer A and Isomer B, and a second stereocenter is then introduced into one or both of Isomer A and Isomer B to produce a mixture of diastereomers. In such instances, individual stereoisomers may be separated and designated with sequential labels following the designations previously provided. For example, introducing a second stereocenter into Isomer A may produce a mixture of diastereomers, which may then be separated into individual stereoisomers, the first-eluting stereoisomer being labeled “Isomer C,” and the second-eluting isomer being labeled “Isomer D.” See Example A7, which is illustrative. The absolute stereochemistry for “Isomer C” and “Isomer D” may be obtained by known methods. It should also be appreciated that, in Tables A, 1, 2, and 3, references to Isomer A, Isomer B, Isomer C, or Isomer D indicate a final-product stereoisomer from the corresponding synthetic example. For example, in Tables A, 1, 2, and 3, a reference to “Compound 2 (Isomer A, Example A2)” indicates the Isomer A final product of synthetic Example A2.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. Compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table A is depicted with a particular stereochemical configuration, also provided herein is any alternative stereochemical configuration of the compound, as well as a mixture of stereoisomers of the compound in any ratio. For example, where a compound of Table A has a stereocenter that is in an “S” stereochemical configuration, also provided herein is the enantiomer of the compound wherein that stereocenter is in an “R” stereochemical configuration. Likewise, when a compound of Table A has a stereocenter that is in an “R” configuration, also provided herein is enantiomer of the compound in an “S” stereochemical configuration. Also provided are mixtures of the compound with both the “S” and the “R” stereochemical configuration. Furthermore, certain structures may exist as geometric isomers (i.e., cis and trans isomers), as tautomers, or as atropisomers. For example, compounds of any formula given herein may contain bonds with restricted rotation and therefore exist in different geometric configurations. Where a compound is depicted as a particular geometric isomer (e.g., E or Z isomer, or cis or trans isomer), also provided herein is any alternative geometric configuration of the compound, as well as a mixture of geometric isomers of the compound in any ratio. For example, where a compound is depicted as a “Z” isomer, also provided herein is the “E” isomer of the compound. Likewise, where a compound is depicted as an “E” isomer, also provided herein is the “Z” isomer of the compound. Also provided are mixtures of the compound with both the “E” and the “Z” stereochemical configuration, wherein the mixtures are in any ratio. Similarly, where a compound is depicted as a “cis” isomer, also provided herein is the “trans” isomer of the compound; and where a compound is depicted as a “trans” isomer, also provided herein is the “cis” isomer of the compound. Also provided are mixtures of the compound with both the “cis” and the “trans” stereochemical configuration, wherein the mixtures are in any ratio. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a pharmaceutically acceptable salt, stereoisomer or tautomer thereof, in a suitable container are provided. The container may be a vial, jar, ampoule, preloaded syringe, I.V. bag, and the like.

Preferably, the compounds detailed herein are orally bioavailable. However, the compounds may also be formulated for parenteral (e.g., intravenous) administration.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

Particular examples are provided in the Example section below. It is understood that the schemes above may be modified to arrive at various compounds of the invention by selection of appropriate reagents and starting materials. For a general description of protecting groups and their use, see P. G. M. Wuts and T. W. Greene, Greene's Protective Groups in Organic Synthesis 4^{th} edition, Wiley-Interscience, New York, 2006.

### Pharmaceutical Compositions and Formulations

Pharmaceutical compositions of any of the compounds detailed herein or a pharmaceutically acceptable salt, stereoisomer or tautomer thereof, are embraced by this disclosure. Thus, the present disclosure includes pharmaceutical compositions comprising a compound as detailed herein or a pharmaceutically acceptable salt, stereoisomer or tautomer thereof, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein or a pharmaceutically acceptable salt, stereoisomer or tautomer thereof, may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein or a pharmaceutically acceptable salt, stereoisomer or tautomer thereof, and a pharmaceutically acceptable carrier. In another variation, methods of administering a compound are provided. The purified forms, pharmaceutical compositions and methods of administering the compounds are suitable for any compound or form thereof detailed herein.

A compound detailed herein or a pharmaceutically acceptable salt, stereoisomer or tautomer thereof, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a pharmaceutically acceptable salt, stereoisomer or tautomer thereof, can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds or a pharmaceutically acceptable salt, stereoisomer or tautomer thereof, as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein or a pharmaceutically acceptable salt, stereoisomer or tautomer thereof, can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use and Uses

Compounds and compositions detailed herein, such as a pharmaceutical composition containing a compound of any formula provided herein or a pharmaceutically acceptable salt, stereoisomer or tautomer thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided is a method of treating a disease in an individual in need thereof comprising administering to the individual a therapeutically effective amount of a compound of formula (I), or any variation thereof, e.g., a compound of formula (I), (IIa), (IIb), (IIIa), (IIIb), (IVa), or (IVb), a compound selected from the compounds depicted in Table A, or a pharmaceutically acceptable salt, stereoisomer or tautomer thereof. In one aspect, the individual is a human. In some variations, a compound is a stereoisomer thereof.

Arginase is implicated in various pathological states. In some embodiments, a compound of formula (I), (IIa), (IIb), (IIIa), (IIIb), (IVa), or (IVb), a compound selected from the compounds depicted in Table A, or a pharmaceutically acceptable salt, stereoisomer or tautomer thereof can be used in a number of therapeutic applications, including but not limited to; psoriasis, septic shock, vascular diseases, airway hyper-responsiveness and rheumatoid arthritis, pulmonary hypertension, hypertension, T cell dysfunction, erectile dysfunction, atherosclerosis, renal disease, ischemia and reperfusion injury, neurodegenerative disease, wound healing, inflammatory disease and fibrotic disease. In some embodiments, given the role of arginase in chronic inflammation and suppression of anti-tumor immunity, a compound of formula (I), (IIa), (IIb), (IIIa), (IIIb), (IVa), or (IVb), a compound selected from the compounds depicted in Table A, or a pharmaceutically acceptable salt, stereoisomer or tautomer thereof can be used in combination with checkpoint blockade, adoptive T cell therapy, CAR-T cells, adoptive NK cell therapy, and the chemotherapy agent gemcitabine.

In some embodiments, the invention provides a method for the treatment or prevention of a disease or condition associated with expression or activity of arginase I, arginase II, or a combination thereof in an individual.

In certain embodiments, the disease or condition is selected from cardiovascular disorders, sexual disorders, wound healing disorders, gastrointestinal disorders, autoimmune disorders, immune disorders, infections, pulmonary disorders and hemolytic disorders.

In certain embodiments, the disease or condition is a cardiovascular disorder selected from systemic hypertension, interstitial lung disease, pulmonary arterial hypertension (PAH), pulmonary arterial hypertension in high altitude, ischemia reperfusion (IR) injury, myocardial infarction, and atherosclerosis. In certain embodiments, the disease or condition is pulmonary arterial hypertension (PAH).

In certain embodiments, the disease or condition is myocardial infarction or atherosclerosis.

In certain embodiments, the disease or condition is a pulmonary disorder selected from chemically-induced lung fibrosis, idiopathic pulmonary fibrosis, cystic fibrosis, chronic obstructive pulmonary disease (COPD), and asthma.

In certain embodiments, the disease or condition is an autoimmune disorder selected from encephalomyelitis, multiple sclerosis, anti-phospholipid syndrome 1, autoimmune hemolytic anaemia, chronic inflammatory demyelinating polyradiculoneuropathy, dermatitis herpetiformis, dermatomyositis, myasthenia gravis, pemphigus, rheumatoid arthritis, stiff-person syndrome, type 1 diabetes, ankylosing spondylitis, paroxysmal nocturnal hemoglobinuria (PNH), paroxysmal cold hemoglobinuria, severe idiopathic autoimmune hemolytic anemia, and Goodpasture's syndrome.

In certain embodiments, the disease or condition is an immune disorder selected from myeloid-derived suppressor cell (MDSC) mediated T-cell dysfunction, human immunodeficiency virus (HIV), autoimmune encephalomyelitis, and ABO mismatch transfusion reaction.

In certain embodiments, the disease or condition is myeloid-derived suppressor cell (MDSC) mediated T-cell dysfunction.

In certain embodiments, the disease or condition is a hemolytic disorder selected from sickle-cell disease, thalassemias, hereditary spherocytosis, stomatocytosis, microangiopathic hemolytic anemias pyruvate kinase deficiency, infection-induced anemia, cardiopulmonary bypass and mechanical heart valve-induced anemia, and chemical induced anemia.

In certain embodiments, the disease or condition is a gastrointestinal disorder selected from gastrointestinal motility disorders, gastric cancer, inflammatory bowel disease, Crohn's disease, ulcerative colitis, and gastric ulcer.

In certain embodiments, the disease or condition is a sexual disorder selected from Peyronie's Disease and erectile dysfunction.

In certain embodiments, the disease or condition is an infection selected from a parasitic infection, a viral infection, and a bacterial infection. In certain embodiments, the bacterial infection is tuberculosis. In certain embodiments, the disease or condition is ischemia reperfusion (IR) injury selected from liver IR, kidney IR, and myocardial IR.

In certain embodiments, the disease or condition is selected from renal disease inflammation, psoriasis, leishmaniasis, neurodegenerative diseases, wound healing, human immunodeficiency virus (HIV), hepatitis B virus (HBV), H. pylori infections, fibrotic disorders, arthritis, candidiasis, periodontal disease, keloids, adenotonsillar disease, African sleeping sickness and Chagas' disease.

In certain embodiments, the disease or condition is a wound healing disorder selected from infected and uninfected wound healing.

Without being bound to any particular theory, arginase inhibitors, such as compounds and compositions detailed herein may promote an anti-tumor immune response by restoring arginine levels, thereby allowing activation of the body's cytotoxic T-cells. In some embodiments, compounds and compositions detailed herein can be used in treating or preventing cancer.

In certain embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Extracranial Germ Cell Tumor, Extragonadal Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is a variety of acute myeloid leukemia (AML), bladder cancer, breast cancer, colorectal cancer, chronic myelogenous leukemia (CML), esophageal cancer, gastric cancer, lung cancer, melanoma, mesothelioma, non-small cell lung carcinoma (NSCLC), ovarian cancer, pancreatic cancer, prostate cancer, renal cancer or skin cancer.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, compounds and compositions detailed herein can be used in treating or preventing an immunological disease.

In certain embodiments, the immunological disease is selected from ankylosing spondylitis, Crohn's disease, erythema nodosum leprosum (ENL), graft versus host disease (GVHD), HIV-associated wasting syndrome, lupus erythematosus, organ transplant rejection, post-poly cythemia, psoriasis, psoriatic arthritis, recurrent aphthous ulcers, rheumatoid arthritis (RA), severe recurrent aphthous stomatitis, systemic sclerosis, and tuberous sclerosis.

In certain embodiments, the method for treating or preventing an immunological disease further comprises conjointly administering an immuno-oncology therapeutic agent.

In some embodiments, compounds and compositions detailed herein can be used in treating or preventing a chronic infection.

In certain embodiments, the chronic infection is selected from bladder infection, chronic fatigue syndrome, cytomegalovirus/epstein barr virus, fibromyalgia, hepatitis B virus (HBV), hepatitis C virus (HCV), HIV/AIDS virus, mycoplasma infection, and urinary tract infections.

In certain embodiments, the method for treating or preventing a chronic infection further comprises conjointly administering an immuno-oncology therapeutic agent.

In some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease associated with arginase.

### Combination Therapy

As provided herein, compounds or pharmaceutically acceptable salt, stereoisomer or tautomer thereof described herein and compositions described herein may be administered with an agent to treat any of the diseases and disorders disclosed herein.

In certain embodiments, the method of treatment provided herein further comprises administering to the subject a therapeutically effective amount of an anti-viral agent, a chemotherapeutic agent including alkylating antineoplastic agents, antimetabolites, anti-microtubule agents (including but not limited to oxaliplatin, gemcitabine, dacarbazine, temozolomide, doxorubicin, 5-fluorouracil), an immunosuppressant (including but not limited to everolimus), immunodulators (including but not limited to check-point inhibitors: anti-PD-1, anti_PD-L1, anti-CTLA4-a antibodies and IDO/TDO inhibitors), radiation, photodynamic therapy, anti-tumor vaccines, oncolytic viruses, antiviral vaccines, cytokine and chemokine therapy or a tyrosine kinase inhibitor, agents affecting interleukins, topoisomerase inhibitors, cytotoxic antibiotic, targeted therapies such as antibodies, antibodies drug conjugates, cell-based immunotherapies, nanoparticles, prior to, simultaneously with, or after administration of at least one compound of the invention, or a stereoisomer, tautomer, pharmaceutically acceptable salt, solvate, or a produg thereof.

Provided herein are methods for treating or preventing a disease (e.g., cancer or a viral infection) by administering to an individual a combination therapy.

In some embodiments, provided are methods of co-administering compounds and compositions detailed herein with an adoptive immunotherapy.

In certain embodiments, compounds and compositions detailed herein are co-administered with an adoptive T-cell immunotherapy or an adoptive NK cell immunotherapy to enhance the efficacy of the adoptive T-cell or NK cell immunotherapy. In some embodiments, the adoptive T-cell immunotherapy involves transfer of cytotoxic T cells (CTLs) such as CD8+ T cells to an individual. In some embodiments, the adoptive T-cell immunotherapy involves transfer of both CD4+ T cells. In some embodiments, the adoptive T-cell immunotherapy involves transfer of both CD8+ T cells and CD4+ T cells to the subject. In some embodiments, the adoptive immunotherapy involves transfer of both T cells and NK cells.

Compounds and compositions detailed herein may enhance the efficacy of the adoptive immunotherapy when administered to an individual (e.g., human) with cancer. In some such embodiments, the cancer is melanoma. In other embodiments, the cancer is multiple myeloma. In other embodiments, the cancer is lung cancer. In other embodiments, the cancer is breast cancer.

The adoptive immunotherapy and compounds and compositions detailed herein may be administered with chemotherapeutic agents. In one such embodiment, a standard-of-care chemotherapeutic agent is gemcitabine. In another such embodiment, the chemotherapeutic agent is cyclophosphamide. In another such embodiment, the chemotherapeutic reagent is fludarabine. The chemotherapeutic reagent(s) can be administered prior to, after and/or concurrently with the adoptive immunotherapy/arginase inhibitor. In all of these embodiments, the adoptive immunotherapy and compounds and compositions detailed herein may be administered with one or more cytokines (e.g., IL-2 or IL-5).

The adoptive immunotherapy and compounds and compositions detailed herein may be administered with one or more immune-modulating agents. For instance, they may be administered with an immune checkpoint inhibitor such as a PD-1 inhibitor, PD-L1 inhibitor or a CTLA-4 inhibitor to enhance the efficacy of the adoptive immunotherapy. In such embodiments, the checkpoint inhibitor can be administered prior to, after and/or concurrently with the adoptive immunotherapy/arginase inhibitor. In all of these embodiments, the adoptive immunotherapy and compounds and compositions detailed herein may be administered with one or more cytokines (e.g., IL-2 or IL-5).

The adoptive immunotherapy and compounds and compositions detailed herein may be administered with one or more inhibitors of the enzyme IDO-1. In certain such embodiments, the IDO-1 inhibitor is epacadostat.

In certain embodiments, the method of treatment further comprises administering to the subject a therapeutically effective amount of PD-1, PD-L1 and/or CTLA-4 antibodies.

In some embodiments, provided are methods of co-administering compounds and compositions detailed herein with an adoptive cell transfer.

In certain embodiments, the adoptive cell transfer involves transferring immune cells (e.g., T cells, such as cytotoxic T cells (CTLs), or natural killer (NK) cells, such as NK-92 cells) to a subject with a disease (e.g., cancer or a viral infection). In some embodiments, the immune cells express chimeric antigen receptors. In some embodiments, the immune cells express a receptor specific for a disease associated peptide. The immune cells may be autologous (i.e., from the subject) or allogenic (i.e., from a donor or from a cell bank). Such immune cells (e.g., T cells, such as CTLs) may be expanded in the presence of antigen presenting cells (APCs) that present one or more disease-specific peptide(s) prior to administration to the subject. The APCs may be B cells, dendritic cells, or artificial antigen-presenting T-cells (aK562 T cells). In some embodiments, the immune cells are not enriched.

In some embodiments, provided are methods of co-administering compounds and compositions detailed herein with immune cells. The composition comprising immune cells may further comprise one or more cytokines (e.g., IL-2 or IL-15). In certain embodiments, about 1×10⁶ cells/kg cells to about 1×10⁹ cells/kg cells are administered to the subject.

In some embodiments, provided are methods of co-administering compounds and compositions detailed herein with an antibody (e.g., an antibody that targets tumor cells). The antibody may be a monoclonal, polyclonal, or a chimeric antibody.

In some embodiments, (a) a compound described herein, or pharmaceutically acceptable salt, stereoisomer or tautomer thereof, or a pharmaceutical composition described herein and (b) an agent are sequentially administered, concurrently administered or simultaneously administered. In certain embodiments, (a) a compound described herein, or a pharmaceutically acceptable salt, stereoisomer or tautomer thereof, or a pharmaceutical composition described herein and (b) an agent are administered with a time separation of about 15 minutes or less, such as about any of 10, 5, or 1 minutes or less. In certain embodiments, (a) a compound described herein, or pharmaceutically acceptable salt, stereoisomer or tautomer thereof, or a pharmaceutical composition described herein and (b) an agent are administered with a time separation of about 15 minutes or more, such as about any of 20, 30, 40, 50, 60, or more minutes. Either (a) a compound described herein, or pharmaceutically acceptable salt, stereoisomer or tautomer thereof, or a pharmaceutical composition described herein and (b) an agent may be administered first. In certain embodiments, (a) a compound described herein, or pharmaceutically acceptable salt, stereoisomer or tautomer thereof, or a pharmaceutical composition described herein and (b) an agent are administered simultaneously.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account factors, e.g., the mode or route of administration or drug delivery, the pharmacokinetics of the agent, the severity and course of the disease to be treated, the subject's health status, condition, and weight. An exemplary dose is in the range of about from about 0.1 mg to 10 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a pharmaceutically acceptable salt, stereoisomer or tautomer thereof may be administered to an individual via various routes, including, e.g., intravenous, intramuscular, subcutaneous, oral, and transdermal. In some embodiments, the compound or composition is administered orally. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

Also provided herein are compositions (including pharmaceutical compositions) as described herein for the use in treating, preventing, and/or delaying the onset and/or development of a disease described herein and other methods described herein. In certain embodiments, the composition comprises a pharmaceutical formulation which is present in a unit dosage form.

### Articles of Manufacture and Kits

The present disclosure further provides articles of manufacture comprising a compound of the disclosure or a salt thereof, composition, and unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the disclosure, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of disease described herein, such as cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein (e.g., hypertension) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Synthetic Examples

The following examples are offered to illustrate but not to limit the present disclosure. One of skill in the art will recognize that the following synthetic reactions and schemes may be modified by choice of suitable starting materials and reagents in order to access other compounds of Formula (I), (IIa), (IIb), (IIIa), (IIIb), (IVa), or (IVb), or pharmaceutically acceptable salt, stereoisomer or tautomer thereof. The compounds are prepared using the general methods described above.

In some embodiments, a pair of stereoisomers (including, for example, a pair of enantiomers or diastereomers) may be separated by any suitable method, including, but not limited to, chiral HPLC. When a pair of stereoisomers is separated by HPLC, it is to be appreciated that the resultant individual stereoisomers will be assigned sequential labels (e.g., A, B, etc.), the order of which implies the order in which the isomers eluted from the HPLC column. For example, when a pair of stereoisomers is separated by HPLC, it is to be appreciated that the first-eluting isomer is labeled “Isomer A,” and the second-eluting isomer is labeled “Isomer B.” The absolute stereochemistry for “Isomer A” and “Isomer B” may be obtained by known methods. In other embodiments, more than one stereocenter is present in a molecule, such as when a first compound containing a first stereocenter is separated into Isomer A and Isomer B, and a second stereocenter is then introduced into one or both of Isomer A and Isomer B to produce a mixture of diastereomers. In such instances, individual stereoisomers may be separated and designated with sequential labels following the designations previously provided. For example, introducing a second stereocenter into Isomer A may produce a mixture of diastereomers, which may then be separated into individual stereoisomers, the first-eluting stereoisomer being labeled “Isomer C,” and the second-eluting isomer being labeled “Isomer D.” See Example A7, which is illustrative. The absolute stereochemistry for “Isomer C” and “Isomer D” may be obtained by known methods. It should also be appreciated that, in Tables A, 1, 2, and 3, references to Isomer A, Isomer B, Isomer C, or Isomer D indicate a final-product stereoisomer from the corresponding synthetic example. For example, in Tables A, 1, 2, and 3, a reference to “Compound 2 (Isomer A, Example A2)” indicates the Isomer A final product of synthetic Example A2.

### Example A1: Synthesis of Compound 1

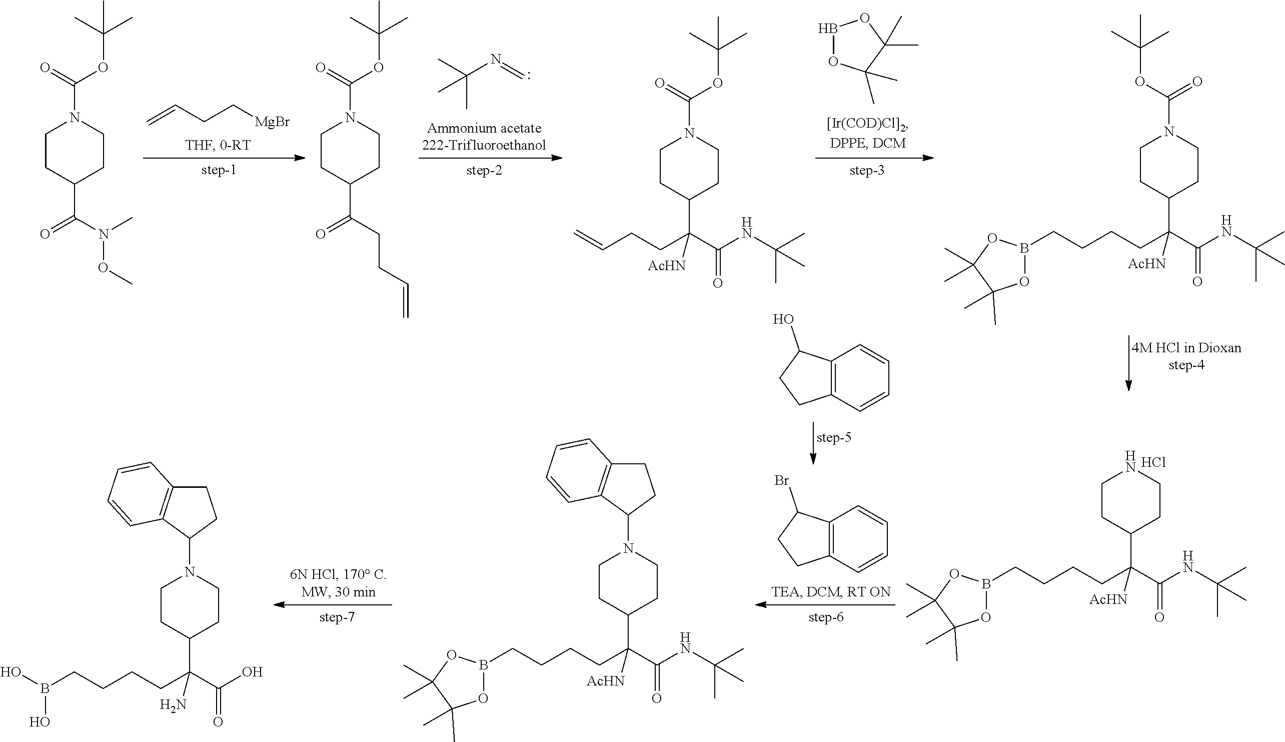

### Step-1: Synthesis of tert-butyl 4-(pent-4-enoyl)piperidine-1-carboxylate

To the stirred solution of tert-butyl 4-(methoxy(methyl)carbamoyl)piperidine-1-carboxylate (1000 mg, 3.67 mmol) in THF (10 ml) was added but-3-enylmagnesium bromide (22 ml, 22.02 mmol). The reaction mixture was allowed to stir at RT for 8 h. Product formation was monitored by TLC and NMR. After completion of reaction the reaction mixture was quenched by 1 N citric acid (50 mL) and extracted with ethyl acetate (100 mL×3). Combined organic layer was washed with brine (100 mL), dried over anhydrous Na₂SO₄ and concentrated under vacuo. The crude product thus obtained was purified by flash chromatography (0-40% ethyl acetate in hexane as an eluent) to obtain tert-butyl 4-(pent-4-enoyl)piperidine-1-carboxylate (950 mg, 92% yield) as a transparent oil. This reaction was performed in 2 batches of 1 g each resulting in a total yield of (0.95 g+0.95 g=1.8 g). LCMS: 268.3 [M+H]⁺.

¹H NMR (400 MHz, CDCl₃): δ 5.83-5.73 (m, 1H), 5.04-4.95 (m, 2H), 4.13-4.07 (m, 2H), 2.76 (t, J=12 Hz, 2H), 2.54 (t, J=8 Hz, 2H), 2.49-2.41 (m, 1H), 2.34-2.28 (m, 2H), 1.85-1.76 (m, 2H), 1.56-1.49 (m, 1H), 1.44 (s, 9H), 1.28-1.24 (m, 1H).

### Step-2: Synthesis of tert-butyl 4-(2-acetamido-1-(tert-butylamino)-1-oxohex-5-en-2-yl)piperidine-1-carboxylate

To a solution of tert-butyl 4-pent-4-enoylpiperidine-1-carboxylate (1800 mg, 6.74 mmol) in trifluoroethanol (15 mL) was added ammonium acetate (2075 mg, 26.96 mmol) and tert-butyl isocyanide (1.5 mL, 13.48 mmol). The resultant reaction mixture was stirred for 4 days at room temperature. The reaction progress was monitored by TLC & NMR. After completion of reaction, the mixture was concentrated under vacuo to afford crude reaction mixture. The reaction mixture was diluted with water (150 mL) and extracted using ethyl acetate (400 mL). Organic layer was dried over anhydrous Na₂SO₄ and concentrated under reduced pressure. The crude product was purified by flash chromatography (0-50% ethyl acetate in hexane as an eluent) to obtain of tert-butyl 4-(2-acetamido-1-(tert-butylamino)-1-oxohex-5-en-2-yl)piperidine-1-carboxylate (1320 mg, 47.7% Yield) as a transparent oil. LCMS: 410.5 [M+H]⁺.

¹H NMR (400 MHz, DMSO-d₆): δ 7.26 (s, 1H), 6.93 (s, 1H), 5.80-5.68 (m, 1H), 5.01-4.87 (m, 2H), 4.08-3.92 (m, 2H), 2.56-2.55 (m, 2H), 2.09-1.92 (m, 2H), 1.89 (s, 3H), 1.86-1.79 (m, 1H), 1.75-1.69 (m, 3H), 1.37 (s, 9H), 1.25 (s, 9H), 1.10-1.07 (m, 3H).

### Step-3: Synthesis of tert-butyl 4-(2-acetamido-1-(tert-butylamino)-1-oxo-6-(4,4,5,5-tetramethyl-1,3,2-dioxaborolan-2-yl)hexan-2-yl)piperidine-1-carboxylate

To a stirred solution of tert-butyl 4-(2-acetamido-1-(tert-butylamino)-1-oxohex-5-en-2-yl)piperidine-1-carboxylate (1300 mg, 3.17 mmol, 1.0 eq.) in DCM (15 mL) was added [Ir(COD)Cl]₂ (64 mg, 0.095 mmol, 0.03 eq.) and DPPE (76 mg, 0.19 mmol, 0.06 eq.). The reaction mixture was allowed to stir at RT for 15 min then cooled to 0° C. 4,4,5,5-tetramethyl-1,3,2-dioxaborolane (0.7 mL, 4.76 mmol, 1.5 eq.) was added drop wise at 0° C. After completion of addition the mixture was allowed to stir at RT for overnight. Reaction progress was monitored by TLC and NMR. The reaction mixture was diluted with ethyl acetate (100 mL) and washed with brine (50 mL). Organic layer was separated and dried over anhydrous Na₂SO₄ and concentrated under reduced pressure. The crude product obtained was purified by flash chromatography (0-50% ethyl acetate in hexane as an eluent) to obtain tert-butyl 4-(2-acetamido-1-(tert-butylamino)-1-oxo-6-(4,4,5,5-tetramethyl-1,3,2-dioxaborolan-2-yl)hexan-2-yl)piperidine-1-carboxylate (1600 mg, 94% yield) as a yellow semi solid. LCMS: 538.5 [M+H]⁺.

### Step-4: Synthesis of 2-acetamido-N-(tert-butyl)-2-(piperidin-4-yl)-6-(4,4,5,5-tetramethyl-1,3,2-dioxaborolan-2-yl)hexanamide hydrochloride

To a stirred solution of tert-butyl 4-(2-acetamido-1-(tert-butylamino)-1-oxo-6-(4,4,5,5-tetramethyl-1,3,2-dioxaborolan-2-yl)hexan-2-yl)piperidine-1-carboxylate (200 mg, 0.48 mmol) in DCM (5 mL) was added 4 N HCl in dioxane (5 mL). The reaction mixture was allowed to stir at RT overnight. Product formation was confirmed by LCMS and NMR. The reaction mixture was concentrated under reduced pressure to obtain 2-acetamido-N-(tert-butyl)-2-(piperidin-4-yl)-6-(4,4,5,5-tetramethyl-1,3,2-dioxaborolan-2-yl)hexanamide hydrochloride which was directly used for next step without any purification. LCMS: 438.5 [M+H]⁺.

### Step-5: Synthesis of 1-bromo-2,3-dihydro-1H-indene

To a solution of 2,3-dihydro-1H-inden-1-ol (1000 mg, 7.46 mmol, 1.0 eq.) in DCM (12 mL) was added phosphorous tribromide (1.6 mL, 16.88 mmol, 2 eq.) in DCM (3 mL) drop wise at 0° C. After completion of addition the mixture was allowed to stir at RT for 3 h. Product formation was confirmed by NMR. After completion of reaction the reaction mixture was quenched by 5% solution of Na₂CO₃ in water and extracted with DCM (50 mL×3). Combined organic layer was dried over anhydrous Na₂SO₄ and concentrated under reduced pressure to obtain 1-bromo-2,3-dihydro-1H-indene (800 mg, 54.2% yield).

¹H NMR (400 MHz, CDCl₃-d): δ 7.32-7.57 (m, 1H), 7.11-7.32 (m, 3H), 5.60 (dd, J=2.19, 6.14 Hz, 1H), 3.18 (s, 1H), 2.69-2.42 (m, 1H), 2.57 (dd, J=6.80, 14.69 Hz, 2H).

### Step-6: Synthesis of 2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide

To a stirred solution of 2-acetamido-N-(tert-butyl)-2-(piperidin-4-yl)-6-(4,4,5,5-tetramethyl-1,3,2-dioxaborolan-2-yl)hexanamide hydrochloride (200 mg, 0.42 mmol, 1.0 eq.) in DCM (5 mL) was added triethylamine (170 mg, 1.68 mmol, 4.0 eq.) at 0° C. The reaction mixture was allowed to stir at RT for 10 min. 1-bromo-2,3-dihydro-1H-indene (83 mg, 0.42 mmol, 1.0 eq.) was added and the reaction mixture was stirred at RT for 16 h. Reaction progress was monitored by LCMS. Reaction was quenched by adding water and extracted with DCM (40 mL×3). Combined organic layer was dried over anhydrous Na₂SO₄ and concentrated under reduced pressure to obtain 2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide (200 mg as a crude) which was directly used for next step without any purification. LCMS: 554.6 [M+H]⁺.

### Step-7: Synthesis of 2-amino-6-borono-2-(1-(2,3-dihydro-1H-inden-1-yl)piperidin-4-yl)hexanoic acid

2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide (200 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous layer was separated and freeze dried using a lyophilizer. The crude product was purified by reverse phase HPLC to obtain 2-amino-6-borono-2-(1-(2,3-dihydro-1H-inden-1-yl)piperidin-4-yl)hexanoic acid TFA salt (10 mg) as an off-white solid. LCMS: 375.4 [M+H]⁺ & 357.3 [M−H₂O]⁺.

¹H NMR (400 MHz, D₂O) δ 7.57 (d, J=7.5 Hz, 1H), 7.54-7.42 (m, 2H), 7.40-7.32 (m, 1H), 4.94 (d, J=7.9 Hz, 1H), 3.57 (s, 1H), 3.42 (s, 1H), 3.35 (s, 2H), 3.18-3.09 (m, 1H), 3.06-2.93 (m, 2H), 2.59-2.37 (m, 3H), 2.18-2.00 (m, 2H), 1.97-1.76 (m, 3H), 1.39 (dt, J=7.0, 13.8 Hz, 2H), 1.28-1.11 (m, 2H), 0.86-0.70 (m, 2H).

### Example A2: Synthesis of Compound 2 (Isomer A)

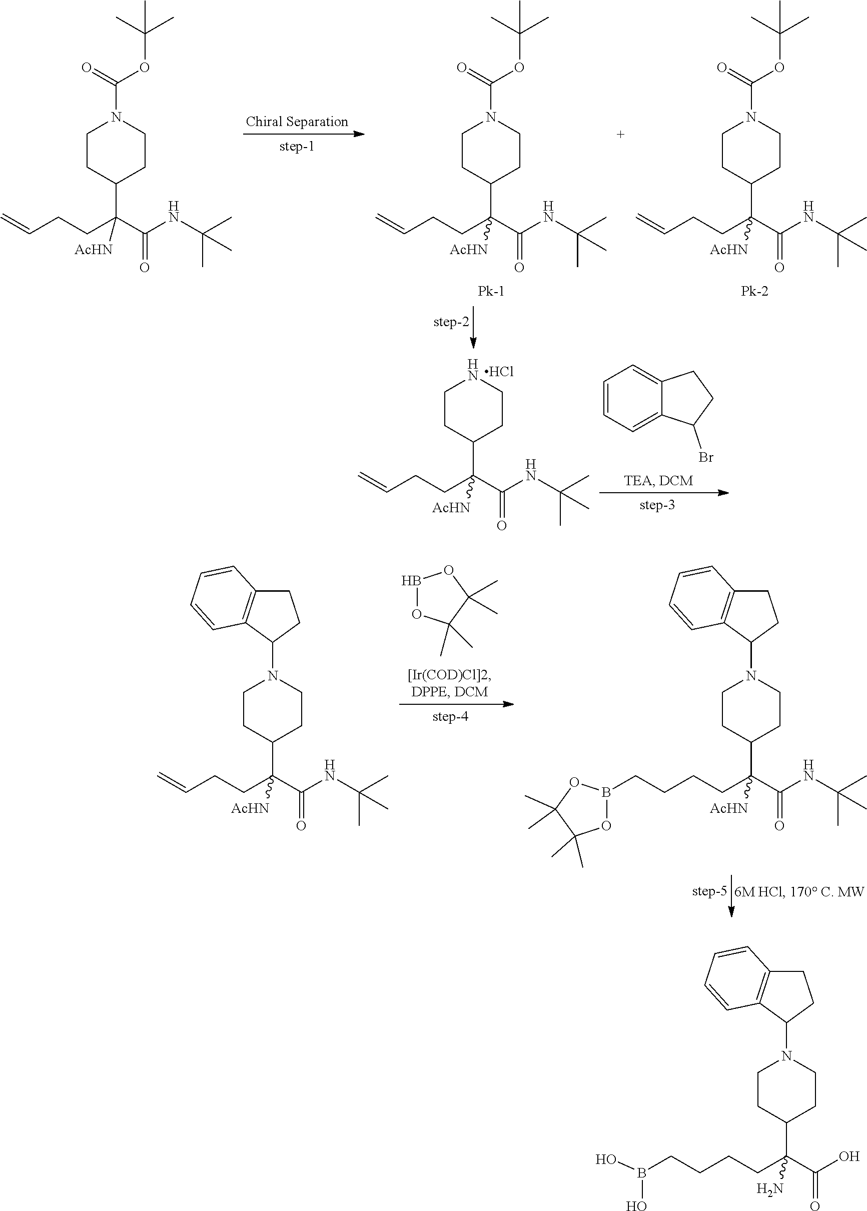

### Step-1: Chiral separation of tert-butyl-4-(2-acetamido-1-(tert-butylamino)-1-oxohex-5-en-2-yl)piperidine-1-carboxylate

Chiral resolution method: The enantiomers were separated by chiral SFC (Chiralcel-ODH, 250×4.6 mm, 5μ) Isocratic Program with analytical grade liquid carbon dioxide and HPLC grade EtOH (0.2% DEA).

### Step-2: 2-acetamido-N-(tert-butyl)-2-(piperidin-4-yl)hex-5-enamide hydrochloride Isomer A

To a stirred solution of tert-butyl-4-(2-acetamido-1-(tert-butylamino)-1-oxohex-5-en-2-yl)piperidine-1-carboxylate Isomer A (350 mg, 0.86 mmol) was added 4 N HCl in dioxane (10 mL). The reaction mixture was allowed to stir at RT for 2 h. Product formation was confirmed by LCMS and NMR. The reaction mixture was concentrated under reduced pressure and freeze dried to obtain 2-acetamido-N-(tert-butyl)-2-(piperidin-4-yl)hex-5-enamide hydrochloride Isomer A (290 mg 97% yield) which was directly used for next step. LCMS: 310.3 [M+H]±.

¹H NMR (400 MHz, DMSO-d₆) δ 8.82 (br.s, 1H), 8.44 (br.s, 1H), 7.38 (s, 1H), 6.96 (s, 1H), 5.83-5.70 (m, 1H), 4.99-4.90 (m, 2H), 3.27 (t, J=12 Hz, 2H), 2.84-2.60 (m, 2H), 2.40 (dd, J=8.6, 12 Hz, 1H), 2.15 (t, J=12 Hz, 2H), 2.06-1.93 (m, 1H), 1.91 (s, 3H), 1.88-1.80 (m, 1H), 1.78-1.65 (m, 1H), 1.63-1.52 (m, 1H), 1.52-1.38 (m, 2H), 1.27 (s, 9H).

### Step-3: 2-acetamido-N-(tert-butyl)-2-(1-(2,3-dihydro-1H-inden-1-yl)piperidin-4-yl)hex-5-enamide Isomer A

To a stirred solution of 2-acetamido-N-(tert-butyl)-2-(piperidin-4-yl)hex-5-enamide hydrochloride Isomer A (300 mg, 0.87 mmol, 1.0 eq.) in DCM (5 mL) was added triethylamine (264 mg, 2.61 mmol, 3.0 eq.) at 0° C. The reaction mixture was allowed to stir at RT for 10 min. 1-bromo-2,3-dihydro-1H-indene (514 mg, 2.61 mmol, 3.0 eq.) was added and the reaction mixture was stirred at RT for 16 h. The reaction progress was monitored by LCMS. Reaction was quenched by adding water and extracted with DCM (40 mL×3). Combined organic layer was dried over anhydrous Na₂SO₄ and concentrated under reduced pressure to obtain 2-acetamido-N-(tert-butyl)-2-(1-(2,3-dihydro-1H-inden-1-yl)piperidin-4-yl)hex-5-enamide Isomer A (300 mg) as an off-white solid. LCMS: 426.3 [M+H]±.

¹H NMR (400 MHz, DMSO-d₆) δ 7.24-7.14 (m, 5H), 6.94 (d, J=16 Hz, 1H), 5.79-5.7 (m, 1H), 5.00-4.85 (m, 2H), 4.24 (br. s., 1H), 2.91-2.79 (m, 2H), 2.78-2.63 (m, 1 H), 2.13 (br. s., 2H), 2.03-1.92 (m, 3H), 1.90 (s, 3H), 1.80 (br. s., 2H), 1.70 (br. s., 2H), 1.34-1.29 (m, 3H), 1.25 (s, 9H), 1.22-1.17 (m, 1H), 1.06 (d, J=8 Hz, 1H).

### Step-4: 2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide Isomer A

To a stirred solution of 2-acetamido-N-(tert-butyl)-2-(1-(2,3-dihydro-1H-inden-1-yl)piperidin-4-yl)hex-5-enamide Isomer A (300 mg, 0.71 mmol, 1.0 eq.) in DCM (5 mL) was added [Ir(COD)Cl]₂ (14 mg, 0.02 mmol, 0.03 eq.) and DPPE (16 mg, 0.04 mmol, 0.06 eq.). The reaction mixture was allowed to stir at RT for 15 min then cooled to 0° C. 4,4,5,5-tetramethyl-1,3,2-dioxaborolane (141 mg, 1.10 mmol, 1.5 eq.) was added drop wise at 0° C. After completion of addition the mixture was allowed to stir at RT for overnight. Reaction progress was monitored by LCMS. The reaction mixture was diluted with ethyl acetate (100 mL) and washed with brine (50 mL). Organic layer was separated and dried over anhydrous Na₂SO₄ and concentrated under reduced pressure to obtain 2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide Isomer A (250 mg, as a crude) which was directly used for next step. LCMS: 554.4 [M+H]⁺.

### Step-5: 2-amino-6-borono-2-(1-(2,3-dihydro-1H-inden-1-yl)piperidin-4-yl)hexanoic acid Isomer A

2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide Isomer A (250 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-(1-(2,3-dihydro-1H-inden-1-yl)piperidin-4-yl)hexanoic acid Isomer A TFA salt (18 mg) as an off-white solid. LCMS: 375.4 [M+H]⁺ & 357.3 [M+H−H₂O]⁺.

¹H NMR (400 MHz, D₂O) δ 7.57 (d, J=7.5 Hz, 1H), 7.54-7.42 (m, 2H), 7.40-7.32 (m, 1H), 4.94 (d, J=7.9 Hz, 1H), 3.57 (br. s., 1H), 3.42 (br. s., 1H), 3.18-3.09 (m, 2H), 3.06-2.93 (m, 2H), 2.59-2.37 (m, 3H), 2.08 (br. s., 2H), 1.97-1.76 (m, 3H), 1.65-1.46 (m, 1H), 1.44-1.26 (m, 3H), 1.16 (br.s., 1H), 0.75 (t, J=8 Hz, 2H).

### Example A3: Synthesis of Compound 3 (Isomer B)

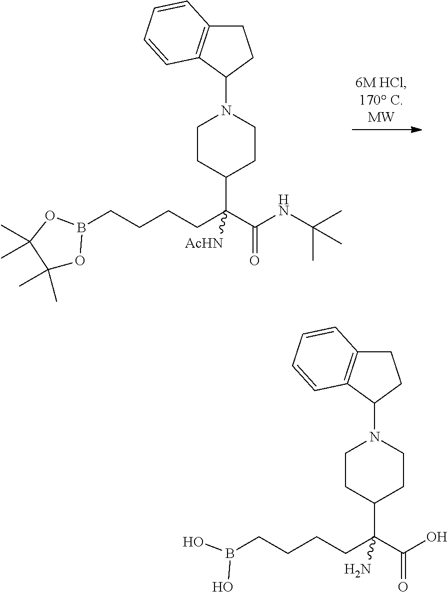 [SMILES: CC(=O)NC(CCCCB1OC(C)(C)C(C)(C)O1)(C(=O)NC(C)(C)C)C1CCN(C2CCc3ccccc32)CC1 ; NC(CCCCB(O)O)(C(=O)O)C1CCN(C2CCc3ccccc32)CC1]

### Synthesis of 2-amino-6-borono-2-(1-(2,3-dihydro-1H-inden-1-yl)piperidin-4-yl)hexanoic acid Isomer B

2-acetamido-N-(tert-butyl)-2-(1-(2,3-dihydro-1H-inden-1-yl)piperidin-4-yl)-6-(4,4,5,5-tetramethyl-1,3,2-dioxaborolan-2-yl)hexanamide Isomer B (300 mg as a crude, synthesized using same conditions as for Isomer A) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reversed phase HPLC to obtain 2-amino-6-borono-2-(1-(2,3-dihydro-1H-inden-1-yl)piperidin-4-yl)hexanoic acid TFA salt Isomer B (18 mg) as an off-white solid. LCMS: 375.4 [M+F1]+& 357.3 [M+H−H₂O]⁺.

¹H NMR (400 MHz, D₂O) δ 7.57 (d, J=8 Hz, 1H), 7.54-7.42 (m, 2H), 7.40-7.32 (m, 1H), 4.94 (d, J=8 Hz, 1H), 3.57 (br.s., 1H), 3.42 (br.s., 1H), 3.35 (s, 2H), 3.18-3.09 (m, 1H), 3.06-2.93 (m, 2H), 2.49-2.37 (m, 3H), 2.18-2.00 (m, 2H), 1.97-1.76 (m, 3H), 1.42-1.39 (m, 2H), 1.28-1.11 (m, 2H), 0.78 (t, J=8 Hz, 2H).

### Example A4: Synthesis of Compound 4

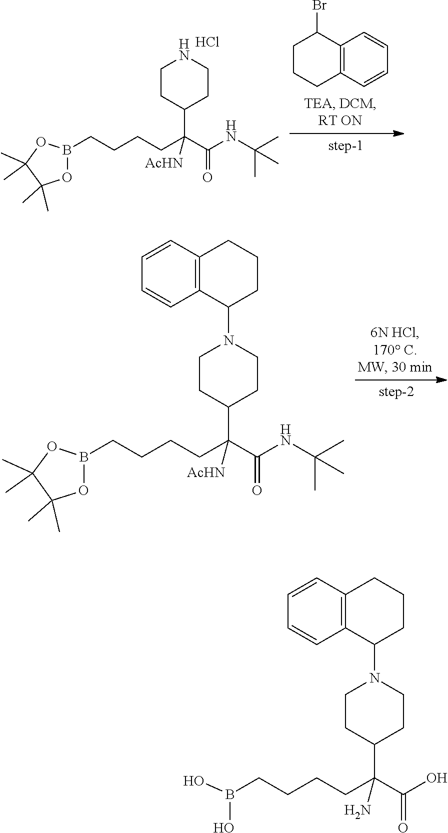

### Step-1: Synthesis of 2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide

To a stirred solution of 2-acetamido-N-(tert-butyl)-2-(piperidin-4-yl)-6-(4,4,5,5-tetramethyl-1,3,2-dioxaborolan-2-yl)hexanamide hydrochloride (200 mg, 0.42 mmol, 1.0 eq.) in DCM (10 mL) was added triethylamine (238 mg, 2.34 mmol, 5.0 eq.) at 0° C. The reaction mixture was allowed to stir at RT for 10 min. 1-bromo-1,2,3,4-tetrahydronaphthalene (146 mg, 0.69 mmol, 1.5 eq.) was added and the reaction mixture was stirred at RT for 16 h. The reaction progress was monitored by LCMS. After completion of reaction it was quenched by adding water and extracted with DCM (40 mL×3). Combined organic layer was dried over anhydrous Na₂SO₄ and concentrated under reduced pressure to obtain 2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide (300 mg as a crude) which was directly used for next step. LCMS: 568.1 [M+H]⁺.

### Step-2: Synthesis of 2-amino-6-borono-2-(1-(1,2,3,4-tetrahydronaphthalen-1-yl)piperidin-4-yl)hexanoic acid

2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide (300 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-(1-(1,2,3,4-tetrahydronaphthalen-1-yl)piperidin-4-yl)hexanoic acid TFA salt (10 mg) as an off-white solid. LCMS: 389.4 [M+H]⁺ & 371.3 [M+H−H₂O]⁺.

¹H NMR (400 MHz, D₂O) δ 7.46-7.40 (m, 2H), 7.32 (t, J=8 Hz, 2H), 3.68-3.63 (br.s., 1H), 3.54-3.51 (br. s., 1H), 3.35 (s, 1H), 3.20-3.03 (m, 2H), 2.9-2.82 (m, 2H), 2.27-2.17 (m, 2H), 2.16-2.07 (m, 2H), 1.88-1.80 (m, 6H), 1.56-1.52 (m, 1H), 1.46-1.38 (m, 3H), 1.22-1.08 (m, 1H), 0.79 (t, J=8 Hz, 2H).

### Example A5: Synthesis of Compound 5 (Isomer A)

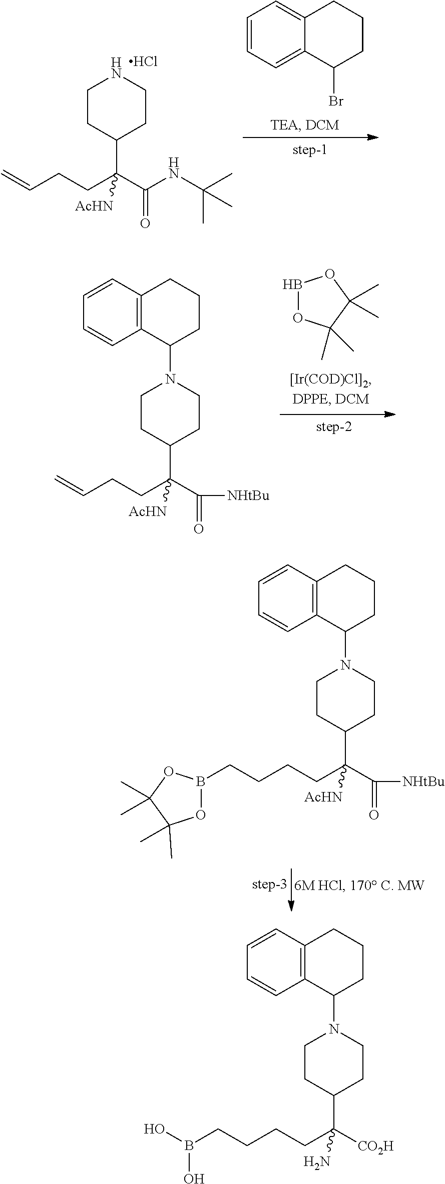

### Step-1: 2-acetamido-N-(tert-butyl)-2-(1-(1,2,3,4-tetrahydronaphthalen-1-yl)piperidin-4-yl)hex-5-enamide Isomer A

To a stirred solution of 2-acetamido-N-(tert-butyl)-2-(piperidin-4-yl)hex-5-enamide hydrochloride Isomer A (1000 mg, 3.20 mmol, 1.0 eq.) in DCM (15 mL) was added triethylamine (653 mg, 6.40 mmol, 2.0 eq.) at 0° C. The reaction mixture was allowed to stir at RT for 10 min. 1-bromo-1,2,3,4-tetrahydronaphthalene (1354 mg, 6.40 mmol, 2.0 eq.) was added and the reaction mixture was stirred at RT for 16 h. The reaction progress was monitored by LCMS. The reaction mixture was concentrated under vacuo, diluted with water (20 mL) extracted with EtOAc (3×100 mL). Combined organic layer was dried over anhydrous Na₂SO₄ and concentrated under vacuo to obtain 2-acetamido-N-(tert-butyl)-2-(1-(1,2,3,4-tetrahydronaphthalen-1-yl) piperidin-4-yl)hex-5-enamide Isomer A (365 mg) as an off-white solid. LCMS: 440.3 [M+H]⁺.

### Step-2: 2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide Isomer A

To a stirred solution of 2-acetamido-N-(tert-butyl)-2-(1-(1,2,3,4-tetrahydronaphthalen-1-yl)piperidin-4-yl)hex-5-enamide Isomer A (200 mg, 0.45 mmol, 1.0 eq.) in DCM (10 mL) was added [Ir(COD)Cl]₂ (9 mg, 0.013 mmol, 0.03 eq.) and DPPE (11 mg, 0.027 mmol, 0.06 eq.). The reaction mixture was allowed to stir at RT for 15 min then cooled to 0° C. 4,4,5,5-tetramethyl-1,3,2-dioxaborolane (0.01 ml, 0.67 mmol, 1.5 eq.) was added drop wise at 0° C. After completion of addition the mixture was allowed to stir at RT for 48 h. Reaction progress was monitored by LCMS. The reaction mixture concentrated up to dry and diluted with ethyl acetate (100 mL) and washed with brine (50 mL). Organic layer was separated and dried over anhydrous Na₂SO₄ and concentrated under reduced pressure to obtain 2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide Isomer A (100 mg, as a crude) which was directly used for next step. LCMS: 568.3 [M+H]⁺.

### Step-3: 2-amino-6-borono-2-(1-(1,2,3,4-tetrahydronaphthalen-1-yl)piperidin-4-yl)hexanoic acid Isomer A

2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide Isomer A (100 mg as a crude) from previous step was dissolved in 6 N HCl (6 mL) and the mixture was heated at 170° C. in microwave reactor for 30 min. Product formation was confirmed by LCMS. The mixture was diluted with water (25 mL) and washed with DCM (10 mL×2). Aqueous phase was separated and freeze dried over lyophilizer. The crude product was purified by reversed phase HPLC to obtain 2-amino-6-borono-2-(1-(1,2,3,4-tetrahydronaphthalen-1-yl)piperidin-4-yl)hexanoic acid Isomer A TFA salt (20 mg) as an off-white solid. LCMS: 389.4 [M+H]⁺ & 371.3 [M+H−H₂O]⁺.

¹H NMR (400 MHz, D₂O) δ 7.40 (d, J=7.5 Hz, 1H), 7.44 (d, J=8 Hz, 1H), 7.36-7.27 (m, 2H), 3.61 (br.s., 1H), 3.47 (br.s., 1H), 3.21-3.09 (m, 2H), 2.92-2.78 (m, 2H), 2.28-2.09 (m, 4H) 2.01-1.75 (m, 6H), 1.72-1.53 (m, 2H), 1.50-1.33 (m, 3H), 1.24-1.15 (m, 1H), 0.79 (t, J=8 Hz, 2H).

### Example A6: Synthesis of Compound 6 (Isomer B)

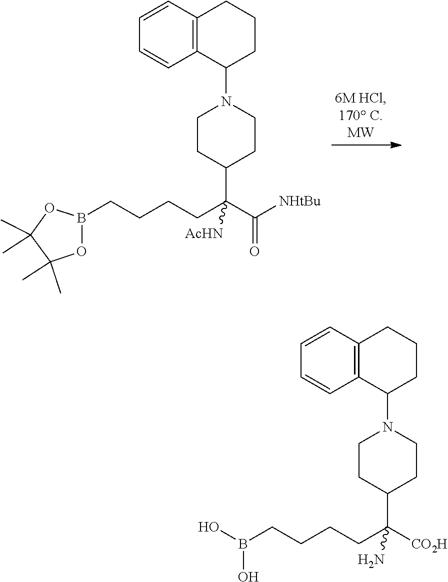

### Synthesis of 2-amino-6-borono-2-(1-(1,2,3,4-tetrahydronaphthalen-1-yl)piperidin-4-yl)hexanoic acid Isomer B

2-acetamido-N-(tert-butyl)-2-(1-(1,2,3,4-tetrahydronaphthalen-1-yl)piperidin-4-yl)-6-(4,4,5,5-tetramethyl-1,3,2-dioxaborolan-2-yl)hexanamide Isomer B (100 mg as a crude) from previous step (synthesized using the same conditions as Isomer B) was dissolved in 6 N HCl (6 mL) and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-(1-(1,2,3,4-tetrahydronaphthalen-1-yl)piperidin-4-yl)hexanoic acid TFA salt Isomer B (9 mg) as an off-white solid. LCMS: 389.4 [M+H]⁺ & 371.3 [M+H−H₂O]⁺.

¹H NMR (400 MHz, D₂O) δ 8.44 (s, 1H, formate), 7.46-7.36 (m, 2H), 7.31 (t, J=8 Hz, 2H), 3.56 (br.s., 1H), 3.42 (br.s., 1H), 3.2-2.98 (m, 2H), 2.93-2.75 (m, 2H), 2.3-2.02 (m, 4H), 2.0-1.73 (m, 6H), 1.67-1.51 (m, 2H), 1.47-1.35 (m, 3H), 1.25-1.11 (m, 1H), 0.83-0.75 (m, 2H).

### Example A7: Synthesis of Compound 7 (Isomer C) and Compound 8 (Isomer D)

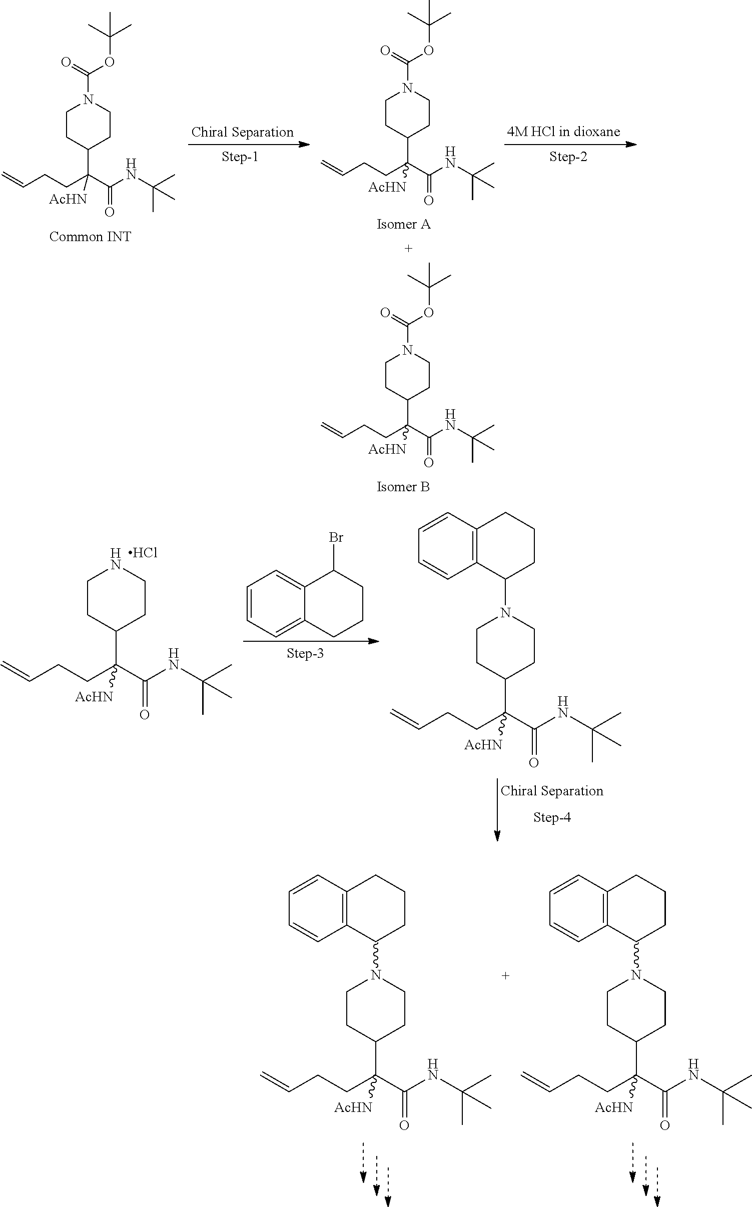

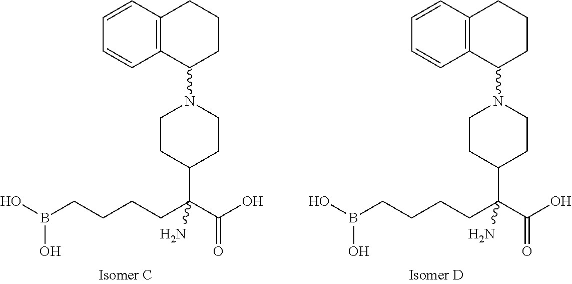 [SMILES: NC(CCCCB(O)O)(C(=O)O)C1CCN(C2CCCc3ccccc32)CC1 ; NC(CCCCB(O)O)(C(=O)O)C1CCN(C2CCCc3ccccc32)CC1]

### Step-1: Chiral Separation of tert-butyl4-(2-acetamido-1-(tert-butylamino)-1-oxohex-5-en-2-yl)piperidine-1-carboxylate

Chiral resolution method: The enantiomers were separated by chiral SFC (Chiralcel-ODH, 250×4.6 mm, 5μ) Isocratic Program with analytical grade liquid carbon dioxide and HPLC grade EtOH (0.2% DEA).

### Step-2: 2-acetamido-N-(tert-butyl)-2-(piperidin-4-yl)hex-5-enamide hydrochloride Isomer A

To a stirred solution of tert-butyl 4-(2-acetamido-1-(tert-butylamino)-1-oxohex-5-en-2-yl)piperidine-1-carboxylate Isomer A (325 mg, 0.8 mmol) was added 4 N HCl in dioxane (10 mL). The reaction mixture was allowed to stir at RT for 2 h. Product formation was confirmed by LCMS and NMR. The reaction mixture was concentrated under reduced pressure and freeze dried to obtain 2-acetamido-N-(tert-butyl)-2-(piperidin-4-yl)hex-5-enamide hydrochloride Isomer A (282 mg) which was directly used for next step. LCMS: 310.3 [M+H]⁺.

### Step-3: 2-acetamido-N-(tert-butyl)-2-(1-(2,3-dihydro-1H-inden-1-yl)piperidin-4-yl)hex-5-enamide Isomer A

To a stirred solution of 2-acetamido-N-tert-butyl-2-(piperidin-4-yl)hex-5-enamide hydrochloride Isomer A (274 mg, 0.79 mmol, 1.0 eq.) in DCM (5 mL) was added triethylamine (240 mg, 2.38 mmol, 3.0 eq.) at 0° C. The reaction mixture was allowed to stir at RT for 10 min. 1-bromo-1,2,3,4-tetrahydronaphthalene (250 mg, 1.19 mmol, 1.5 eq.) was added and the reaction mixture was stirred at RT for 16 h. The reaction progress was monitored by LCMS. Reaction was quenched by adding water and extracted with DCM (40 mL×3). Combined organic layer was dried over anhydrous Na₂SO₄ and concentrated under reduced pressure to obtain 2-acetamido-N-tert-butyl-2-(1-(1,2,3,4-tetrahydronaphthalen-1-yl)piperidin-4-yl)hex-5-enamide Isomer A (160 mg) as an off-white solid. LCMS: 440.3 [M+H]⁺.

### Step-4: Chiral separation of 2-acetamido-N-tert-butyl-2-(1-(1,2,3,4-tetrahydronaphthalen-1-yl)piperidin-4-yl)hex-5-enamide

Chiral resolution method: The diastereomers were separated by chiral SFC (Chiralpak-IA, 250×4.6 mm, 5μ) Isocratic Program with analytical grade liquid carbon dioxide and HPLC grade MeOH (0.2% DEA). Total Flow: 3 gm/min Co-solvent Percentage: 15%, ABPR:100 Bar, Wavelength: 207 nm.

The separated enantiomers were converted to the desired products using the same conditions as for previous examples.

Isomer C ¹H NMR: (400 MHz, D₂O) δ 7.47-7.37 (m, 2H), 7.32 (t, J=8 Hz, 2H), 3.59 (d, J=12 Hz, 1H), 3.47 (d, J=12 Hz, 1H), 3.23-3.06 (m, 2H), 2.95-2.77 (m 2H), 2.26-2.02 (m, 5H), 2.02-1.84 (m, 5H), 1.84-1.74 (m, 1H), 1.65-1.48 (m, 1H), 1.48-1.32 (m, 3H), 1.25-1.15 (m, 1H), 0.76 (t, J=8 Hz, 2H).

Isomer D ¹H NMR: (400 MHz, D₂O) δ 7.40 (d, J=8 Hz, 1H), 7.39-7.31 (m, 1H), 7.25 (t, J=8 Hz, 2H), 3.46 (t, J=12 Hz, 1H), 3.09-2.72 (m, 4H), 2.18-2.08 (br.s., 2H), 2.08-1.87 (m, 4H), 1.84-1.66 (m, 5H), 1.62-1.25 (m, 4H), 1.23-1.08 (m, 2H), 0.75 (t, J=8 Hz, 2H).

### Example A8: Synthesis of Compound 9

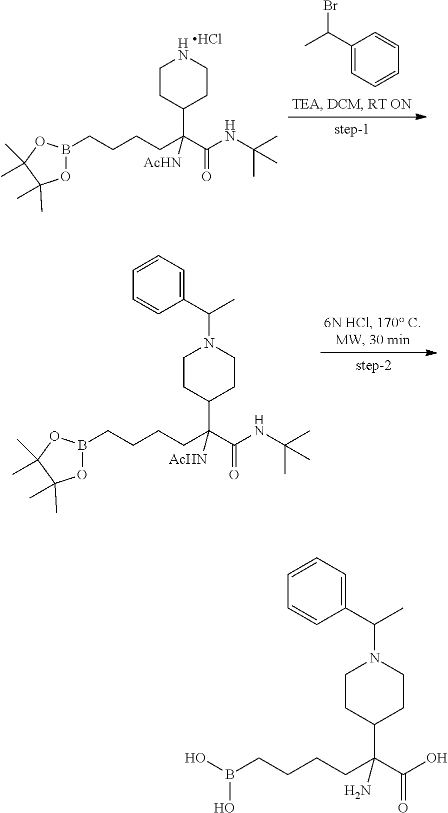

### Step-1: Synthesis of 2-acetamido-N-(tert-butyl)-2-(1-(1-phenylethyl)piperidin-4-yl)-6-(4,4,5,5-tetramethyl-1,3,2-dioxaborolan-2-yl)hexanamide

To a stirred solution of 2-acetamido-N-(tert-butyl)-2-(piperidin-4-yl)-6-(4,4,5,5-tetramethyl-1,3,2-dioxaborolan-2-yl)hexanamide hydrochloride (200 mg, 0.42 mmol, 1.0 eq.) in DCM (4 mL) was added triethylamine (170 mg, 1.68 mmol, 4.0 eq.) at 0° C. The reaction mixture was allowed to stir at RT for 10 min. (1-bromoethyl)benzene (56 mg, 0.3 mmol, 0.7 eq.) was added and the reaction mixture was stirred at RT for 16 h and the reaction progress was monitored by LCMS. The reaction was quenched by adding water and was extracted with DCM (40 mL×3). The organic layer was separated and dried over anhydrous Na₂SO₄ and concentrated under reduced pressure to obtain 2-acetamido-N-(tert-butyl)-2-(1-(1-phenylethyl)piperidin-4-yl)-6-(4,4,5,5-tetramethyl-1,3,2-dioxaborolan-2-yl)hexanamide (200 mg as a crude) which was directly used for next step without any further purification. LCMS: 542.3 [M+H]⁺.

### Step-2: Synthesis of 2-amino-6-borono-2-(1-(1-phenylethyl)piperidin-4-yl)hexanoic acid

2-acetamido-N-(tert-butyl)-2-(1-(1-phenylethyl)piperidin-4-yl)-6-(4,4,5,5-tetramethyl-1,3,2-dioxaborolan-2-yl)hexanamide (260 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-(1-(1-phenylethyl)piperidin-4-yl)hexanoic acid TFA salt (25 mg) as an off-white solid. LCMS: 363.3 [M+H]⁺ & 345.3 [M+H−H₂O]⁺

¹H NMR (400 MHz, D₂O) δ 7.51 (m, 5H), 3.84-3.82 (m, 2H), 3.46-3.43 (m, 1H), 3.01 (s, 1H), 2.93 (s, 2H), 2.86-2.84 (m, 1H), 2.06 (s, 1H), 1.82 (d, J=5.3 Hz, 3H), 1.74 (d, J=7.0 Hz, 2H), 1.44-1.42 (m, 2H), 1.24-1.22 (m, 2H), 1.18-1.86 (m, 2H), 0.81 (t, J=8 Hz, 2H).

### Example A9: Synthesis of Compound 10 (Isomer A) and Compound 11 (Isomer B)

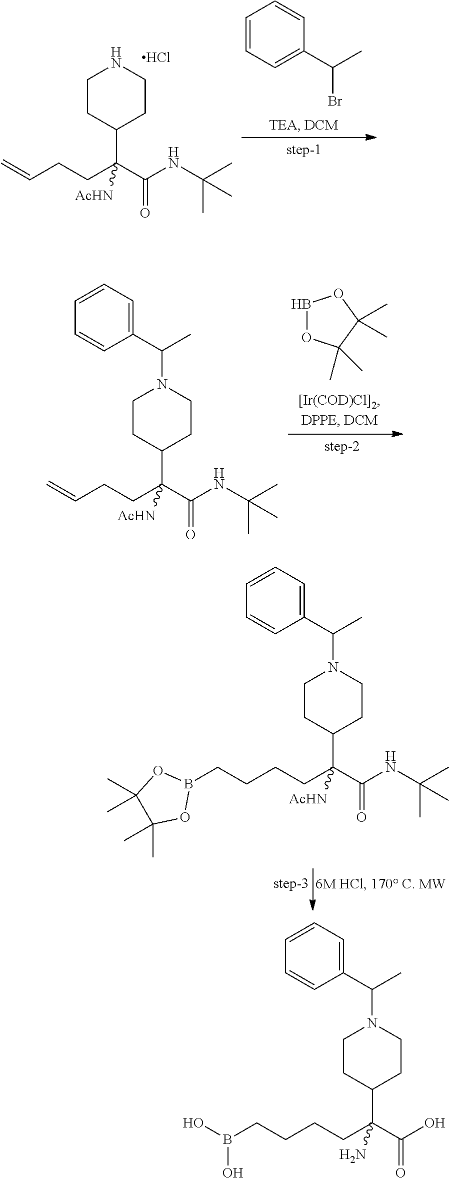

### Step-1: 2-acetamido-N-(tert-butyl)-2-(1-(1-phenylethyl)piperidin-4-yl)hex-5-enamide Isomer A

To a stirred solution of 2-acetamido-N-(tert-butyl)-2-(piperidin-4-yl)hex-5-enamide hydrochloride Isomer A (800 mg, 2.58 mmol, 1.0 eq.) in DCM (20 mL) was added triethylamine (1.0 mL, 7.764 mmol, 3.0 eq.) at 0° C. The reaction mixture was allowed to stir at RT for 10 min. Then (1-bromoethyl)benzene (527 mg, 2.847 mmol, 1.1 eq.) was added and the reaction mixture was stirred at RT for 16 h. The reaction progress was monitored by LCMS. After completion of reaction it was concentrated under vacuo and then diluted with water (20 mL) and extracted with EtOAc (3×100 mL). Combined organic layer was dried over anhydrous Na₂SO₄ and concentrated under vacuo to obtain 2-acetamido-N-(tert-butyl)-2-(1-(1-phenylethyl)piperidin-4-yl)hex-5-enamide Isomer A (365 mg) as an off-white solid. LCMS: 414.3 [M+H]⁺.

### Step-2: 2-acetamido-N-(tert-butyl)-2-(1-(1-phenylethyl)piperidin-4-yl)-6-(4,4,5,5-tetramethyl-1,3,2-dioxaborolan-2-yl)hexanamide Isomer A

To a stirred solution of 2-acetamido-N-(tert-butyl)-2-(1-(1-phenylethyl)piperidin-4-yl)hex-5-enamide (320 mg, 0.77 mmol, 1.0 eq.) in DCM (25 mL) was added [Ir(COD)Cl]₂ (24 mg, 0.038 mmol, 0.05 eq.) and DPPE (31 mg, 0.077 mmol, 0.1 eq.). The reaction mixture was allowed to stir at RT for 15 min then cooled to 0° C. 4,4,5,5-tetramethyl-1,3,2-dioxaborolane (297 mg, 2.324 mmol, 3.0 eq.) was added drop wise at 0° C. After completion of addition the mixture was allowed to stir at RT for 48 h. Reaction progress was monitored by LCMS. The reaction mixture concentrated under vacuo and then diluted with ethyl acetate (100 mL) and washed with brine (50 mL). Organic layer was separated and dried over anhydrous Na₂SO₄ and concentrated under reduced pressure to obtain 2-acetamido-N-(tert-butyl)-2-(1-(1-phenylethyl)piperidin-4-yl)-6-(4,4,5,5-tetramethyl-1,3,2-dioxaborolan-2-yl)hexanamide Isomer A (680 mg, as a crude) which was directly used for next step without any further purification. LCMS: 542.3 [M+H]⁺.

### Step-3: 2-amino-6-borono-2-(1-(1-phenylethyl)piperidin-4-yl)hexanoic acid Isomer A

2-acetamido-N-(tert-butyl)-2-(1-(1-phenylethyl)piperidin-4-yl)-6-(4,4,5,5-tetramethyl-1,3,2-dioxaborolan-2-yl)hexanamide Isomer A (680 mg as a crude) from previous step was dissolved in 6 N HCl (6 mL) and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-(1-(1-phenylethyl)piperidin-4-yl)hexanoic acid Isomer A (TFA salt) (65 mg) as an off-white solid. LCMS: 363.3 [M+H]⁺ & 345.3 [M−H₂O]⁺

¹H NMR (400 MHz, D₂O) δ 7.51 (d, J=7.0 Hz, 5H), 4.45-4.42 (m, 2H), 3.79 (br.s., 1H), 3.44 (br.s., 1H), 3.05-2.8 (m, 2H), 2.22-2.12 (m, 5H), 1.82 (d, J=5.3 Hz, 3H), 1.74-1.42 (m, 4H), 1.18 (br.s., 1H), 0.78 (t, J=8 Hz, 2H).

### Synthesis of 2-amino-6-borono-2-(1-(1-phenylethyl)piperidin-4-yl)hexanoic acid Isomer B

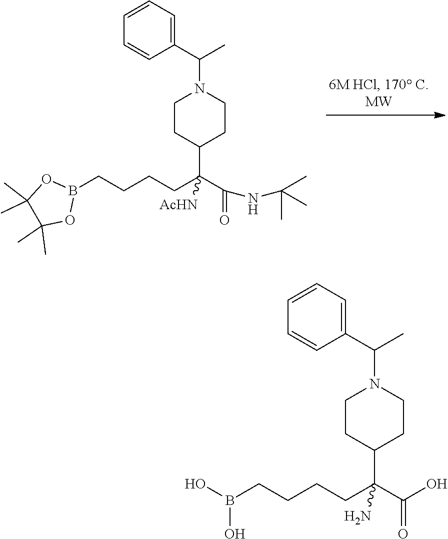 [SMILES: CC(=O)NC(CCCCB1OC(C)(C)C(C)(C)O1)(C(=O)NC(C)(C)C)C1CCN(C(C)c2ccccc2)CC1 ; CC(c1ccccc1)N1CCC(C(N)(CCCCB(O)O)C(=O)O)CC1]

2-acetamido-N-(tert-butyl)-2-(1-(1-phenylethyl)piperidin-4-yl)-6-(4,4,5,5-tetramethyl-1,3,2-dioxaborolan-2-yl)hexanamide Isomer B (600 mg as a crude) from previous step (synthesize using the same conditions as for Isomer A) was dissolved in 6N HCl and the mixture was heated at 170° C. in microwave reactor for 30 min. Product formation was confirmed by LCMS. The mixture was diluted with water (25 mL) and washed with DCM (20 mL×3). Aqueous phase was separated and freeze dried over lyophilizer. The crude product was purified by reversed phase HPLC to obtain 2-amino-6-borono-2-(1-(1-phenylethyl)piperidin-4-yl)hexanoic acid TFA salt Isomer B (5 mg) as an off-white solid. LCMS: 363.3 [M+H]⁺ & 345.3 [M−H₂O]⁺.

¹H NMR (400 MHz, D₂O) δ 7.53-7.51 (m, 5H), 4.48-4.42 (m, 2H), 3.82 (br.s., 1H), 3.47 (br.s., 1H), 3.04-2.9 (m, 1H), 2.9-2.76 (m, 1H), 2.21-1.95 (m, 2H), 1.95-1.78 (m, 3H), 1.76 (d, J=8 Hz, 3H), 1.49-1.29 (m, 4H), 1.26-1.12 (m, 1H), 0.79 (t, J=8 Hz, 2H).

### Example A10: Synthesis of Compound 14 (Isomer A)

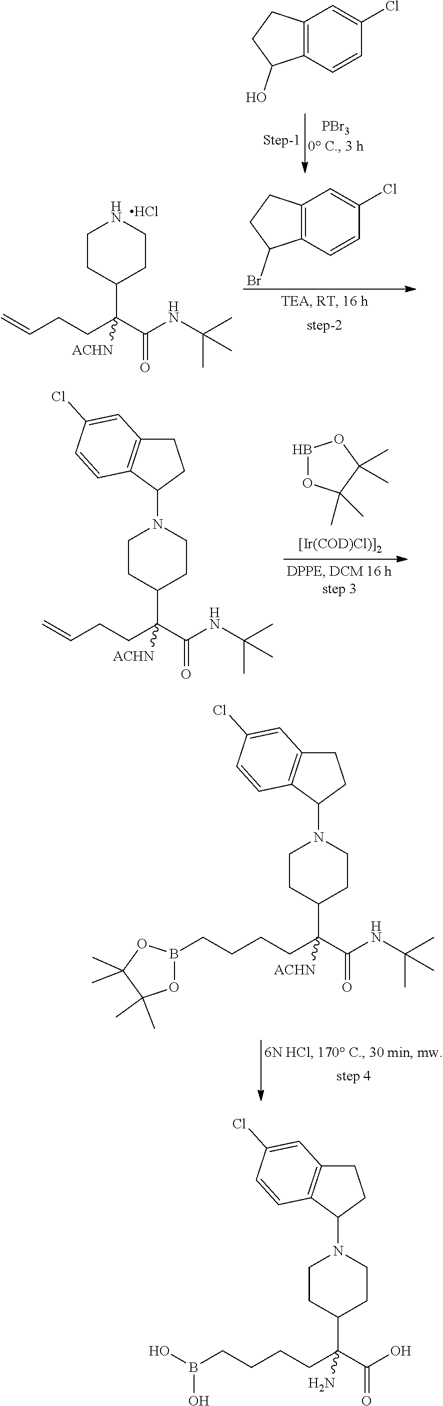

### Step-1: Synthesis of 1-bromo-5-chloro-indane

To a solution of 5-chloroindan-1-ol (600 mg, 3.5 mmol, 1.0 eq.) in DCM (5 mL) was added phosphorous tribromide (0.43 mL, 4.64 mmol, 1.3 eq.) in DCM (3 mL) drop wise at 0° C. After completion of addition the mixture was allowed to stir at RT for 3 h. Product formation was confirmed by NMR. After completion of reaction the reaction mixture was quenched by 5% solution of Na₂CO₃ in water and extracted with DCM (50 mL×3). Combined organic layer was dried over anhydrous Na₂SO₄ and concentrated under reduced pressure to obtain 1-bromo-5-chloro-indane.

¹H NMR: (400 MHz, CHLOROFORM-d) δ ppm 7.33 (d, J=8 Hz, 1H) 7.27-7.18 (m, 2H), 5.51 (dd, J=4, 2 Hz, 1H), 3.14 (p, J=8 Hz, 1H), 2.84 (ddd, J=16, 8, 4 Hz, 1H), 2.67-2.47 (m, 2H).

### Step-2: Synthesis of 2-acetamido-N-tert-butyl-2-[1-(5-chloroindan-1-yl)-4-piperidyl]hex-5-enamide Isomer A

To a stirred solution of 2-acetamido-N-tert-butyl-2-(4-piperidyl)hex-5-enamide hydrochloride Isomer A (300 mg, 0.86 mmol, 1.0 eq.) in DCM (6 mL) was added triethylamine (300 mg, 1.72 mmol, 2 eq.) at 0° C. The reaction mixture was allowed to stir at RT for 10 min and then 1-bromo-5-chloro-indane (312 mg, 1.72 mmol, 1.3 eq.) was added and the reaction mixture was stirred at RT for 16 h. The progress of the reaction was monitored by LCMS. The reaction was quenched by addition of water and extracted with DCM (40 mL×3). Combined organic layer was dried over anhydrous Na₂SO₄ and concentrated under reduced pressure to obtain 2-acetamido-N-tert-butyl-2-[1-(5-chloroindan-1-yl)-4-piperidyl]hex-5-enamide Isomer A (440 mg as a crude) which was directly used for next step without any further purification. LCMS: 460.3 [M]⁺

Step-3: Synthesis of 2-acetamido-N-tert-butyl-2-[1-(5-chloroindan-1-yl)-4-piperidyl]-6-(4,4,5,5-tetramethyl-1,3,2-dioxaborolan-2-yl)hexanamide Isomer A

To a stirred solution of 2-acetamido-N-tert-butyl-2-[1-(5-chloroindan-1-yl)-4-piperidyl]hex-5-enamide Isomer A (350 mg, 0.76 mmol, 1.0 eq.) in DCM (6 mL) was added [Ir(COD)Cl]₂ (31 mg, 0.046 mmol, 0.06 eq.) and DPPE (9 mg, 0.022 mmol, 0.03 eq.). The reaction mixture was allowed to stir at RT for 15 min then cooled to 0° C. 4,4,5,5-tetramethyl-1,3,2-dioxaborolane (0.15 mL, 1.14 mmol, 1.5 eq.) was added drop wise at 0° C. After completion of addition the mixture was allowed to stir at RT for overnight. Reaction progress was monitored by TLC and NMR. After completion of reaction the reaction mixture was diluted with ethyl acetate (80 mL) and washed with brine (40 mL). Organic layer was separated and dried over anhydrous Na₂SO₄ and concentrated under reduced pressure to get 2-acetamido-N-tert-butyl-2-[1-(5-chloroindan-1-yl)-4-piperidyl]-6-(4,4,5,5-tetramethyl-1,3,2-dioxaborolan-2-yl)hexanamide Isomer A (190 mg, as crude) which was directly used for next step without any further purification. LCMS: 588.4 [M]⁺

### Step-4: Synthesis of 2-amino-6-borono-2-[1-(5-chloroindan-1-yl)-4-piperidyl]hexanoic acid Isomer A

To a stirred solution of 2-acetamido-N-tert-butyl-2-[1-(5-chloroindan-1-yl)-4-piperidyl]-6-(4,4,5,5-tetramethyl-1,3,2-dioxaborolan-2-yl)hexanamide Isomer A (190 mg as a crude) from previous step was dissolved in 6 N HCl and the mixture was heated at 170° C. in microwave reactor for 30 min. Product formation was confirmed by LCMS. After completion of reaction the mixture was diluted with water (25 mL) and washed with DCM (15 mL×2). Aqueous phase was separated and freeze dried using lyophilizer. The crude product was purified by reverse phase HPLC to obtain 2-amino-6-borono-2-[1-(5-chloroindan-1-yl)-4-piperidyl]hexanoic acid Isomer A TFA salt (40 mg) as an off-white solid. LCMS: [M+H−H₂O]⁺391.2, [M+H−2H₂O]⁺373.2.

¹H NMR: (400 MHz, D₂O) δ 7.52 (d, J=8.2 Hz, 1H), 7.47 (d, J=2.1 Hz, 1H), 7.38 (dd, J=8.1, 2.1 Hz, 1H), 3.57 (t, J=8 Hz, 1H), 3.42 (t, J=8 Hz, 1H), 3.19-3.06 (m, 2H), 3.05-2.9 (m, 2H), 2.64-2.5 (m, 1H), 2.49-2.39 (m, 1H), 2.17-2.04 (m, 3H), 1.99-1.77 (m, 4H), 1.67-1.47 (m, 1H), 1.47-1.29 (m, 3H), 1.27-1.12 (m, 1H), 0.79 (t, J=8 Hz, 2H).

### Example A11: Synthesis of Compound 16

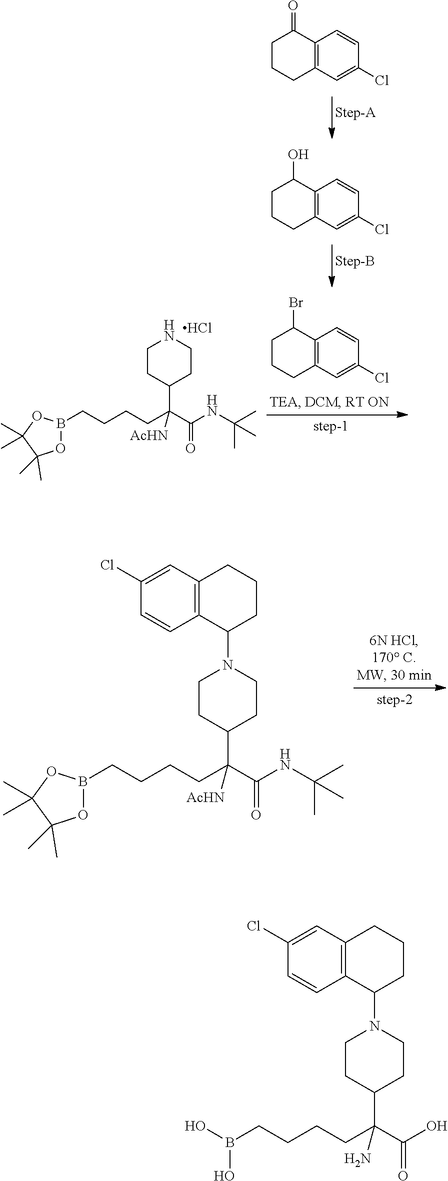

### Step-A: Synthesis of 6-chloro-1-tetralol

To the stirred solution of 6-chloro-tetralone (1 g, 5.53 mmol, 1 eq.) in methanol (10 mL) was added NaBH₄ (315 mg, 8.29 mmol, 1.5 eq.) portion wise at 0° C. under nitrogen atmosphere. The reaction mixture was stirred for 1 h, at RT under nitrogen atmosphere. After the completion of reaction (TLC monitoring) the reaction mass was evaporated under reduced pressure to give crude reaction mixture. The crude reaction mixture was then further diluted with water (150 mL) and extracted with ethyl acetate (150 mL). The organic layer was separated, dried over anhydrous sodium sulphate, filtered and evaporated to give product as yellow oil. LCMS: 183.7 [M+H]⁺.

### Step-B: Synthesis of 1-bromo-6-chloro-tetralin

To the stirred solution of 6-chloro-1-tetranol (1.2 g, 6.9 mmol, 1 eq.) in DCM (10 mL) was added PBr₃ (1 mL, 7.9 mmol, 1.2 eq.) drop wise at 0° C. under nitrogen atmosphere. The reaction mixture was stirred at RT for 3 h under same conditions. After the completion of reaction (TLC monitoring) the reaction mass was evaporated under reduced pressure to give crude reaction mixture. The crude reaction mixture was then diluted with water (100 mL) and extracted with ethyl acetate (100 mL) the organic layer was separated, dried over anhydrous sodium sulphate, filtered and evaporated under reduced pressure to yield the desired product which was used in the next step without any further purification. LCMS: 245.4 [M]⁺.

### Step-1: Synthesis of 2-acetamido-N-tert-butyl-2-(1-(6-chloro-1,2,3,4-tetrahydronaphthalen-1-yl)piperidin-4-yl)-6-(4,4,5,5-tetramethyl-1,3,2-dioxaborolan-2-yl)hexanamide

a stirred solution of 2-acetamido-N-tert-butyl-2-(piperidin-4-yl)-6-(4,4,5,5-tetramethyl-1,3,2-dioxaborolan-2-yl)hexanamide hydrochloride (200 mg, 0.422 mmol) in DCM (4 mL) was added triethylamine (0.11 mL, 0.844 mmol) at 0° C., the reaction mixture was allowed to stir at RT for 10 min then added 1-bromo-6-chloro-1,2,3,4-tetrahydronaphthalene (125 mg, 0.506 mmol) and the reaction mixture was stirred at RT for 16 h. The reaction progress was monitored by LCMS. Reaction was quenched by diluting it with water (50 mL) and extracted with DCM (50 mL×2). The organic layer was separated, dried over anhydrous Na₂SO₄ and concentrated under reduced pressure to obtain 2-acetamido-N-tert-butyl-2-(1-(6-chloro-1,2,3,4-tetrahydronaphthalen-1-yl)piperidin-4-yl)-6-(4,4,5,5-tetramethyl-1,3,2-dioxaborolan-2-yl)hexanamide (200 mg as crude) which was directly taken for next step without any purification. LCMS: 602.4 [M+H]⁺.

The crude from last step 2-acetamido-N-tert-butyl-2-(1-(6-chloro-1,2,3,4-tetrahydronaphthalen-1-yl)piperidin-4-yl)-6-(4,4,5,5-tetramethyl-1,3,2-dioxaborolan-2-yl)hexanamide (200 mg) was dissolved in 6N HCl and subjected to microwave irradiation for 30 minutes at 170° C. The progress of the reaction was monitored by LCMS. The mixture was diluted with water (25 mL) and washed with DCM (10 mL×2). The aqueous layer was separated and lyophilized to give crude product which was purified through reverse phase HPLC to obtain final product 2-amino-6-borono-2-(1-(6-chloro-1,2,3,4-tetrahydronaphthalen-1-yl)piperidin-4-yl)hexanoic acid as free base (off white solid, 4.5 mg). LCMS: 405.2 [M+H−H₂O]⁺.

¹H NMR (400 MHz, D₂O) δ 7.42 (d, J=8.0 Hz, 1H), 7.32-7.29 (m, 2H), 3.62-3.52 (m, 1H), 3.50-3.41 (m, 1H), 3.19-3.01 (m, 2H), 2.94-2.78 (m, 2H), 2.3-2.05 (m, 4H), 2.02-1.9 (m, 3H), 1.89-1.74 (m, 4H), 1.64-1.49 (m, 1H), 1.48-1.29 (m, 3H), 1.25-1.18 (m, 1H), 0.80 (t, J=8 Hz, 2H).

### Example A12: Synthesis of Compound 17 (Isomer A)

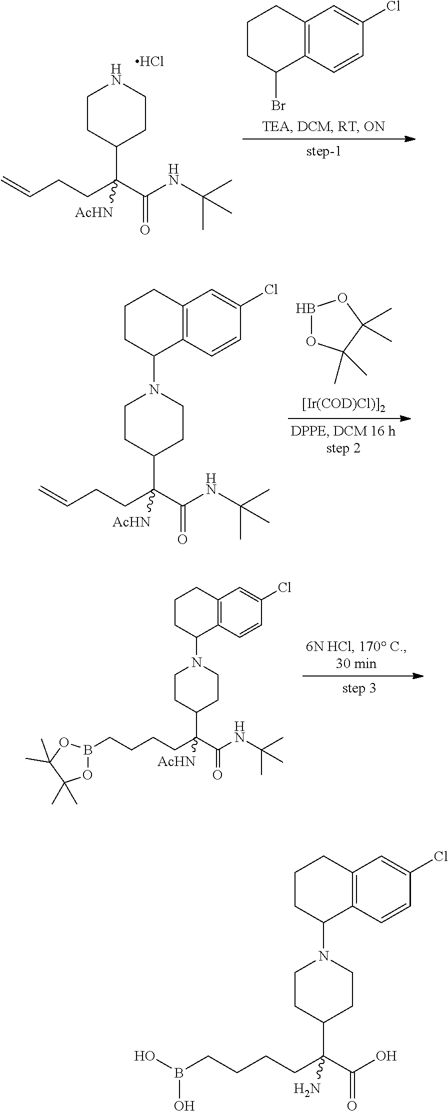

### Step-1: 2-Acetamido-N-tert-butyl-2-(1-(6-chloro-1,2,3,4-tetrahydronaphthalen-1-yl)piperidin-4-yl)hex-5-enamide) Isomer A

To the stirred solution of 1-bromo-6-chloro-1,2,3,4-tetrahydronaphthalene (300 mg, 0.86 mmol) in DCM (5 mL) was added TEA (0.4 mL, 2.58 mmol) and the reaction mixture was stirred for 10 minutes at RT. 2-acetamido-N-tert-butyl-2-(piperidin-4-yl)hex-5-enamide hydrochloride Isomer A (320 mg, 1.30 mmol) was then added to the reaction mixture. After the completion of reaction (LCMS monitoring) the reaction mass was diluted with water (50 mL) and extracted with ethyl acetate (50 mL). The organic layer was separated, dried over anhydrous Na₂SO₄, filtered and evaporated under reduce pressure to get crude product which was used as such in next step without any purification. This reaction was performed in 2 batches of 300 mg each resulting in a total yield of (500 mg+150 mg=650 mg). LCMS: 474.3 [M]⁺

### Step-2: 2-Acetamido-N-tert-butyl-2-(1-(6-chloro-1,2,3,4-tetrahydronaphthalen-1-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(6-chloro-1,2,3,4-tetrahydronaphthalen-1-yl)piperidin-4-yl)hex-5-enamide) Isomer A (500 mg, 1.06 mmol) in DCM (10 mL) was added [Ir(COD)Cl]₂ (42 mg, 0, 0.064 mmol) and DPPE (50 mg, 0.127 mmol) at RT under nitrogen atmosphere and the reaction mass was stirred for 10 minutes. After 10 minutes of stirring at RT the reaction mass was cooled to 0° C. and pinacolborane (0.18 mL, 1.272 mmol) was then added drop wise to the reaction mixture. After the completion of reaction mixture (LCMS monitoring), the reaction mixture was diluted with water (25 mL) and extracted with DCM (25 mL). The organic layer was separated, dried over Na₂SO₄, filtered and evaporated under reduced pressure to get crude residue which was taken as such for next step without any further purification. LCMS: 602.5 [M]⁺

### Step-3: 2-Amino-6-borono-2-(1-(6-chloro-1,2,3,4-tetrahydronaphthalen-1-yl)piperidin-4-yl)hexanoic acid Isomer A

To the stirred solution of 2-acetamido-N-tert-butyl-2-(1-(6-chloro-1,2,3,4-tetrahydronaphthalen-1-yl)piperidin-4-yl)-6-(4,4,5,5-tetramethyl-1,3,2-dioxaborolan-2-yl)hexanamide Isomer A (800 mg, 1.32 mmol) in 6 M HCl (3 mL) was stirred in microwave for 30 minutes at 170° C. After the completion of reaction (LCMS monitoring) the reaction mixture was washed with DCM (50 mL) and the aqueous layer was lyophilized to get crude compound which was purified through reverse phase chromatography to get the desired compound as an off white solid (35 mg). LCMS: 405.2 [M+H−H₂O]⁺

¹H NMR: (400 MHz, D₂O) δ 7.41 (d, J=8 Hz, 1H), 7.39-7.27 (m, 2H), 3.65-3.54 (m, 1H), 3.54-3.45 (m, 1H), 3.22-3.04 (m, 2H), 2.94-2.76 (m, 2H), 2.30-2.07 (m, 4H), 2.0-1.72 (m, 6H), 1.67-1.49 (m, 2H), 1.47-1.29 (m, 3H), 1.26-1.12 (m, 1H), 0.79 (t, J=8 Hz, 2H).

### Example A13: Synthesis of Compound 19 (Isomer A)

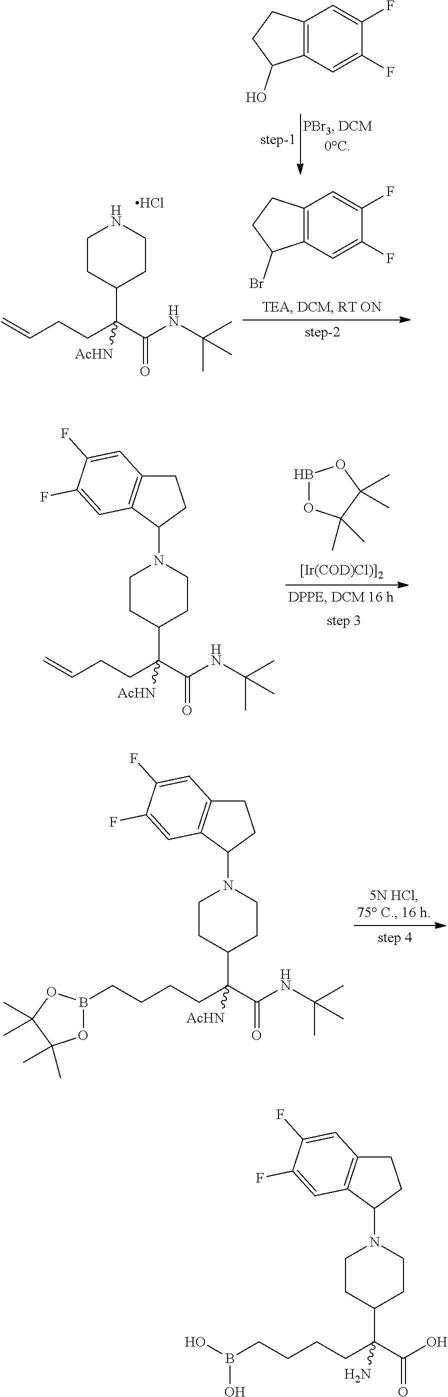

### Step-1: Synthesis of 1-bromo-5,6-difluoro-2,3-dihydro-1H-indene

To a solution of 5,6-difluoro-2,3-dihydro-1H-inden-1-ol (400 mg, 2.35 mmol, 1.0 eq.) in DCM (12 mL) was added phosphorous tribromide (634 mg, 3.52 mmol, 1.5 eq.) in DCM (3 mL) drop wise at 0° C. After completion of addition the mixture was allowed to stir at RT for 3 h. Product formation was confirmed by NMR. After completion of reaction, the reaction mixture was quenched by 5% aqueous solution of Na₂CO₃ and extracted with DCM (50 mL×3). Combined organic layer was dried over anhydrous Na₂SO₄ and concentrated under reduced pressure to yield 1-bromo-5,6-difluoro-2,3-dihydro-1H-indene (600 mg as crude) which was directly taken for next step without any further purification. LCMS: 232.1[M]⁺

### Step-2: Synthesis of 2-acetamido-N-tert-butyl-2-[1-(5,6-difluoroindan-1-yl)-4-piperidyl]hex-5-enamide Isomer A

To a stirred solution of 2-acetamido-N-tert-butyl-2-(4-piperidyl)hex-5-enamide hydrochloride Isomer A (200 mg, 0.579 mmol, 1.0 eq.) in DCM (4 mL) was added triethylamine (117 mg, 1.15 mmol, 2.0 eq.) at 0° C. and the reaction mixture was allowed to stir at RT for 10 min. Then 1-bromo-5,6-difluoro-2,3-dihydro-1H-indene (404 mg, 1.73 mmol, 3.0 eq.) was added to the reaction mixture and it was stirred at RT for 16 h. The reaction progress was monitored by LCMS. Reaction was quenched by diluting it with water (10 mL) and extracted with DCM (50 mL×2). The organic layer was separated, dried over anhydrous Na₂SO₄ and concentrated under reduced pressure to yield 2-acetamido-N-tert-butyl-2-[1-(5,6-difluoroindan-1-yl)-4-piperidyl]hex-5-enamide Isomer A (200 mg as crude) which was directly taken for next step. LCMS: 462.3 [M+H]⁺

### Step 3: Synthesis of 2-acetamido-N-tert-butyl-2-[1-(5,6-difluoroindan-1-yl)-4-piperidyl]-6-(4,4,5,5-tetramethyl-1,3,2-dioxaborolan-2-yl)hexanamide Isomer A

To a stirred solution of 2-acetamido-N-tert-butyl-2-[1-(5,6-difluoroindan-1-yl)-4-piperidyl]hex-5-enamide Isomer A (200 mg, 0.432 mmol, 1.0 eq.) in DCM (10 mL) was added [Ir(COD)Cl]₂ (34 mg, 0.0518 mmol, 0.12 eq.) and DPPE (34 mg, 0.103 mmol 0.24 eq.) and the reaction mixture was stirred for 15 minutes at RT. The reaction mixture was then cooled to 0° C. and 4,4,5,5-tetramethyl-1,3,2-dioxaborolane (165 mg, 1.29 mmol, 3.0 eq.) was added to it. The reaction mixture was allowed to stir overnight at RT. The reaction progress was monitored by LCMS. After completion of reaction, it was quenched by diluting it with water (10 mL) and extracted with DCM (50 mL×2). The organic layer was separated, dried over anhydrous Na₂SO₄ and concentrated under reduced pressure to obtain 2-acetamido-N-tert-butyl-2-[1-(5,6-difluoroindan-1-yl)-4-piperidyl]-6-(4,4,5,5-tetramethyl-1,3,2-dioxaborolan-2-yl)hexanamide Isomer A (300 mg as crude) which was directly taken for next step. LCMS: 590.5 [M+H]⁺

### Step 4: Synthesis of 2-amino-6-borono-2-[1-(5,6-difluoroindan-1-yl)-4-piperidyl]hexanoic acid Isomer A

2-Acetamido-N-tert-butyl-2-[1-(5,6-difluoroindan-1-yl)-4-piperidyl]-6-(4,4,5,5-tetramethyl-1,3,2-dioxaborolan-2-yl)hexanamide Isomer A (300 mg) was dissolved in 5N HCl and the reaction mixture was allowed to stir at 70° C. for 16 h. The progress of the reaction was monitored through LCMS. The mixture was diluted with water (100 mL) and washed with DCM (50 mL×2). The aqueous layer was separated and lyophilized to give crude product which was purified by reverse phase HPLC to obtain final product 2-amino-6-borono-2-[1-(5,6-difluoroindan-1-yl)-4-piperidyl]hexanoic acid Isomer A as formate salt (off white solid, 3 mg). LCMS: 393.2 [M+H−H₂O]⁺

¹H NMR: (400 MHz, D₂O) 7.47 (t, J=8.8 Hz, 1H), 7.30 (t, J=8.9 Hz, 1H), 3.53 (s, 1H), 3.37 (s, 1H), 3.15-2.93 (m, 4H), 2.57 (dd, J=16.1, 8.2 Hz, 1H), 2.07 (d, J=17.0 Hz, 2H), 1.90-1.78 (m, 4H), 1.56 (s, 3H), 1.42 (q, J=7.3 Hz, 1H), 1.34 (d, =9.9 Hz, 2H), 1.19 (d, J=8.2 Hz, 1H), 0.79 (t, J=7.8 Hz, 2H).

### Example A14: Synthesis of Compound 23

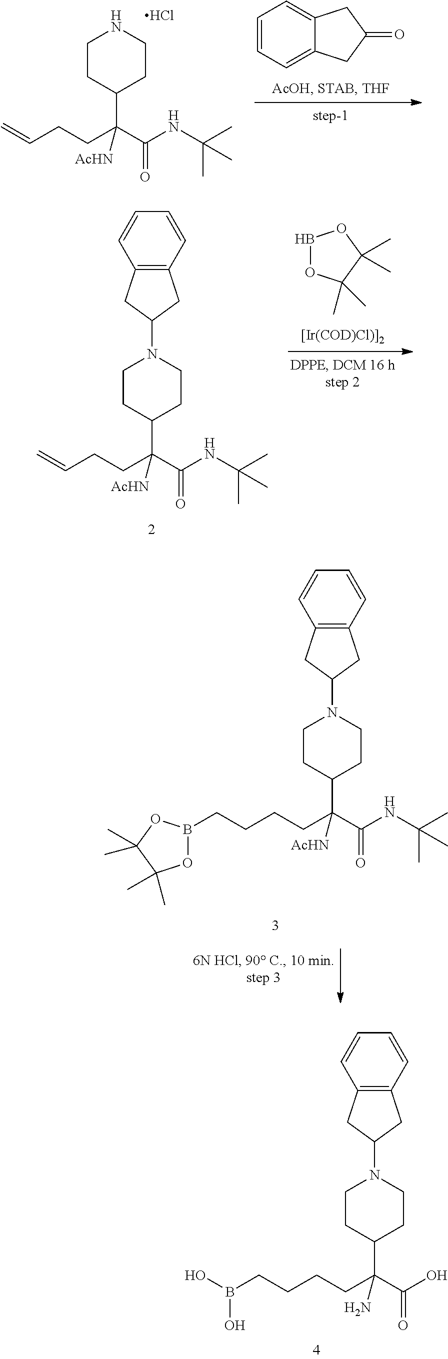

### Step 1: Synthesis of 2-acetamido-N-tert-butyl-2-(1-(2,3-dihydro-1H-inden-2-yl)piperidin-4-yl)hex-5-enamide

To the stirred solution of 2-acetamido-N-tert-butyl-2-(piperidin-4-yl)hex-5-enamide hydrochloride (1 g, 2.89 mmol) in THF (20 mL) was added 1H-inden-2(3H)-one (870 mg, 3.46 mmol) and catalytic amount of AcOH (1-2 drops) and the reaction mixture was allowed to stir for 3 h at RT under nitrogen atmosphere. Sodium triacetoxyborohydride (1.22 g, 5.78 mmol) was then added to the reaction mixture and the reaction mixture was stirred for 16 h at RT under nitrogen atmosphere. After the completion of reaction (LCMS monitoring) the reaction mixture was diluted with water (250 mL) and extracted with ethyl acetate (250 mL). The organic layer was separated, dried over Na₂SO₄, filtered and evaporated under reduced pressure to get crude product which was taken as such for next step without any further purification. LCMS: 426.3 [M+H]⁺

### Step 2: Synthesis of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide

To the stirred solution of 2-acetamido-N-tert-butyl-2-(1-(2,3-dihydro-1H-inden-2-yl)piperidin-4-yl)hex-5-enamide (3 g as crude) in DCM (30 mL) was added [Ir(COD)Cl]₂ (284 mg, 0.42 mmol) and DPPE (340 mg, 0.84 mmol) at RT under nitrogen atmosphere and the reaction mass was stirred for 10 minutes. After 10 minutes of stirring at RT the reaction mass was cooled to 0° C. then added 4,4,5,5-tetramethyl-1,3,2-dioxaborolane (1.23 mL, 8.47 mmol) was added dropwise to the reaction mixture. After the completion of reaction mixture (LCMS monitoring) the reaction mixture was diluted with water (250 mL) and extracted with DCM (250 mL). The organic layer was separated, dried over Na₂SO₄, filtered and evaporated under reduced pressure to get crude residue. The crude was taken as such for next step without any further purification. LCMS: 554.3 [M+H]⁺

### Step 3: Synthesis of 2-amino-6-borono-2-(1-(2,3-dihydro-1H-inden-2-yl)piperidin-4-yl)hexanoic acid

A stirred solution of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide (1 g, 1.79 mmol) in 6 M HCl (3 mL) was stirred in microwave for 10 minutes at 90° C. After the completion of reaction (LCMS monitoring) the reaction mixture was washed with DCM (50 mL) and the aqueous layer was lyophilized to get crude compound which was purified through reverse phase chromatography to get the desired compound as an off white solid (13.5 mg). LCMS: 374.2 [M]⁺

¹H NMR: (400 MHz, D₂O) δ 7.36-7.24 (m, 4H), 4.07 (p, J=7.6 Hz, 1H), 3.62 (d, J=12.7 Hz, 2H), 3.45 (dd, J=16.4, 8.0 Hz, 2H), 3.24-3.13 (m, 2H), 3.02 (t, J=12.8 Hz, 2H), 2.36 (t, J=12.3 Hz, 1H), 2.20-2.00 (m, 3H), 1.90 (dt, J=13.8, 12.9, 4.6 Hz, 1H), 1.71 (m, 1H), 1.60-1.40 (m, 1H), 1.39-1.23 (m, 2H), 1.23-0.99 (m, 2H), 0.84 (t, J=8 Hz, 2H).

### Example A15: Synthesis of Compound 24 (Isomer A1

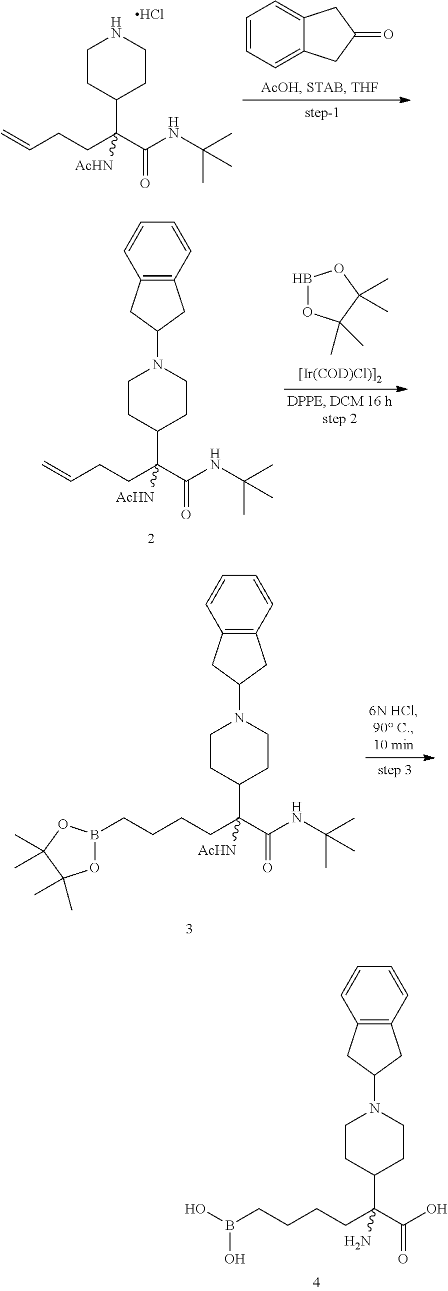

### Step-1: Synthesis of 2-acetamido-N-tert-butyl-2-(1-(2,3-dihydro-1H-inden-2-yl)piperidin-4-yl)hex-5-enamide Isomer A

To the stirred solution of 2-acetamido-N-tert-butyl-2-(piperidin-4-yl)hex-5-enamide hydrochloride Isomer A (150 mg, 0.43 mmol) in THF (5 mL) was added 1H-inden-2(3H)-one (70 mg, 0.52 mmol) and catalytic amount of AcOH (1-2 drops). The resultant reaction mixture was allowed to stir for 3 h at RT under nitrogen atmosphere. Then sodium triacetoxyborohydride (185 mg, 0.86 mmol) was added to the reaction mixture and it was stirred for 16 h at RT under nitrogen atmosphere. After the completion of reaction (LCMS monitoring) the reaction mixture was diluted with water (50 mL) and extracted with ethyl acetate (50 mL). The organic layer was separated, dried over Na₂SO₄, filtered and evaporated under reduced pressure to get crude residue which was used as such for next step without any further purification. LCMS: 426.3 [M+H]⁺

### Step-2: Synthesis of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(2,3-dihydro-1H-inden-2-yl)piperidin-4-yl)hex-5-enamide Isomer A (200 mg as crude) in DCM (3 mL) was added [Ir(COD)Cl]₂ (18 mg, 0.027 mmol) and DPPE (22 mg, 0.055 mmol) at RT under nitrogen atmosphere and the reaction mass was stirred for 10 minutes. After 10 minutes of stirring at RT the reaction mass was cooled to 0° C. and then 4,4,5,5-tetramethyl-1,3,2-dioxaborolane (0.08 mL, 0.56 mmol) was added drop wise to the reaction mixture. After the completion of reaction (LCMS monitoring) the reaction mixture was diluted with water (25 mL) and extracted with DCM (25 mL) the organic layer was separated, dried over Na₂SO₄, filtered and evaporated under reduced pressure to get crude product. The crude was taken as such for next step without any further purification. LCMS: 554.5 [M+H]⁺

### Step-3: Synthesis of 2-amino-6-borono-2-(1-(2,3-dihydro-1H-inden-2-yl)piperidin-4-yl)hexanoic acid Isomer A

A stirred solution of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide Isomer A (500 mg as crude) in 6 M HCl (3 mL) was stirred in microwave for 10 minutes at 90° C. After the completion of reaction (LCMS monitoring) the reaction mixture was diluted with DCM (50 mL) and water (25 mL). The aqueous layer was separated and lyophilized to get crude compound which was purified through reverse phase chromatography to get the desired compound (TFA salt) as an off white solid (15.0 mg). LCMS: 357.1[M+H−H₂O]⁺

¹H NMR: (400 MHz, D₂O) δ 7.38-7.25 (m, 4H), 4.12 (p, J=7.5 Hz, 1H), 3.68 (t, J=10.3 Hz, 2H), 3.47 (dd, J=16.4, 7.9 Hz, 2H), 3.22 (dd, J=16.3, 7.1 Hz, 2H), 3.16-2.97 (m, 2H), 2.17 (m, 2H), 2.02-1.78 (m, 4H), 1.72 (s, 1H), 1.63-1.48 (m, 1H), 1.47-1.29 (m, 2H), 1.25-1.15 (m, 1H), 0.80 (t, J=8 Hz, 2H).

### Example A16: Synthesis of Compound 25 (Isomer B)

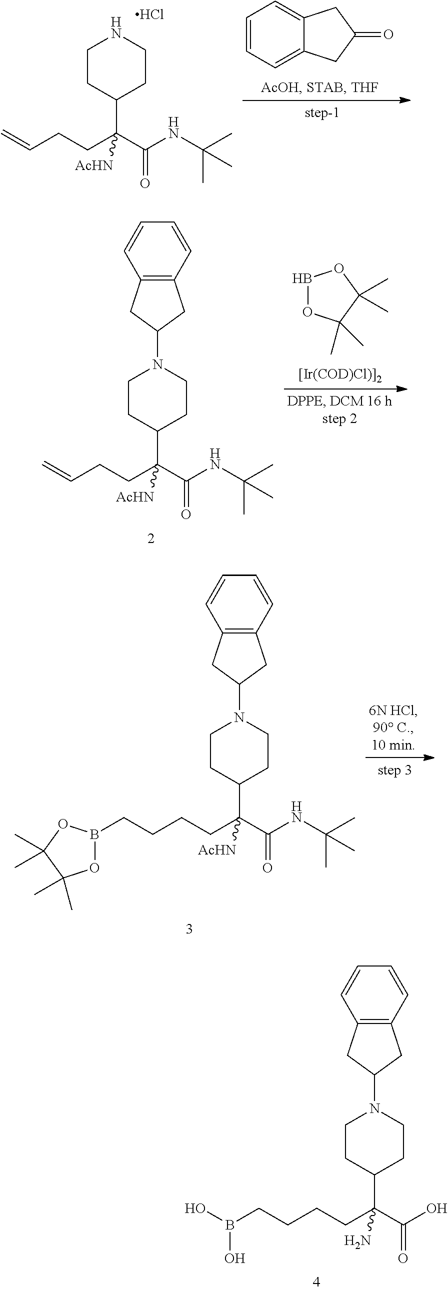

### Synthesis of 2-acetamido-N-tert-butyl-2-(1-(2,3-dihydro-1H-inden-2-yl)piperidin-4-yl)hex-5-enamide Isomer B

To the stirred solution of 2-acetamido-N-tert-butyl-2-(piperidin-4-yl)hex-5-enamide hydrochloride Isomer B (150 mg, 0.43 mmol) in THF (5 mL) was added 1H-inden-2(3H)-one (70 mg, 0.52 mmol) and catalytic amount of AcOH (1-2 drops) the reaction mixture was allowed to stir for 3 h at RT under nitrogen atmosphere. Sodium triacetoxyborohydride (185 mg, 0.86 mmol) was added to the reaction mixture and was stirred for 16 h at RT under nitrogen atmosphere. After the completion of reaction (LCMS monitoring) the reaction mixture was diluted with water (50 mL) and extracted with ethyl acetate (50 mL). The organic layer was separated, dried over Na₂SO₄, filtered and evaporated under reduced pressure to get crude product which was used as such in next step without any further purification. LCMS: 426.4 [M+H]⁺

### Synthesis of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide Isomer B

To the stirred solution of 2-acetamido-N-tert-butyl-2-(1-(2,3-dihydro-1H-inden-2-yl)piperidin-4-yl)hex-5-enamide Isomer B (200 mg as crude) in DCM (3 mL) was added [Ir(COD)Cl]₂ (18 mg, 0.027 mmol) and DPPE (22 mg, 0.055 mmol) at RT under nitrogen atmosphere and the reaction mass was stirred for 10 minutes. After 10 minutes of stirring at RT the reaction mixture was cooled to 0° C. and then 4,4,5,5-tetramethyl-1,3,2-dioxaborolane (0.08 mL, 0.56 mmol) was added drop wise to the reaction mixture. After the completion of reaction, (LCMS monitoring) the reaction mixture was diluted with water (25 mL) and extracted with DCM (25 mL). The organic layer was separated, dried over Na₂SO₄, filtered and evaporated under reduced pressure to get crude product which was taken as such for next step. LCMS: 554.6 [M+H]⁺

### Synthesis of 2-amino-6-borono-2-(1-(2,3-dihydro-1H-inden-2-yl)piperidin-4-yl)hexanoic acid Isomer B

The stirred solution of 2-acetamido-N-tert-butyl-2-(1-(2,3-dihydro-1H-inden-2-yl)piperidin-4-yl)-6-(4,4,5,5-tetramethyl-1,3,2-dioxaborolan-2-yl)hexanamide Isomer B (400 mg as crude) in 6 M HCl (3 mL) was stirred in microwave for 10 minutes at 90° C. After the completion of reaction (LCMS monitoring) the reaction mixture was washed with DCM (50 mL) and the aqueous layer was lyophilized to get crude compound which was purified through reverse phase chromatography to get compound (formate salt) as off white solid (4.34 mg). LCMS: 339.3 [M+H−2H₂O]⁺

¹H NMR: (400 MHz, D₂O) δ 7.74 (d, J=7.3 Hz, 1H), 7.62 (s, 1H), 7.59-7.51 (m, 1H), 7.49 (d, J=6.7 Hz, 1H), 3.64 (br.s., 1H), 3.48 (br.s., 1H), 3.28-3.05 (m, 2H), 3.03-2.85 (m, 2H), 2.33-2.27 (m, 2H), 2.20-2.07 (m, 2H), 2.06-1.74 (m, 4H), 1.72-1.5 (m, 2H), 1.49-1.31 (m, 3H), 1.30-1.12 (m, 1H), 0.81 (t, J=7.7 Hz, 2H).

### Example A17: Synthesis of Compound 32 (Isomer A)

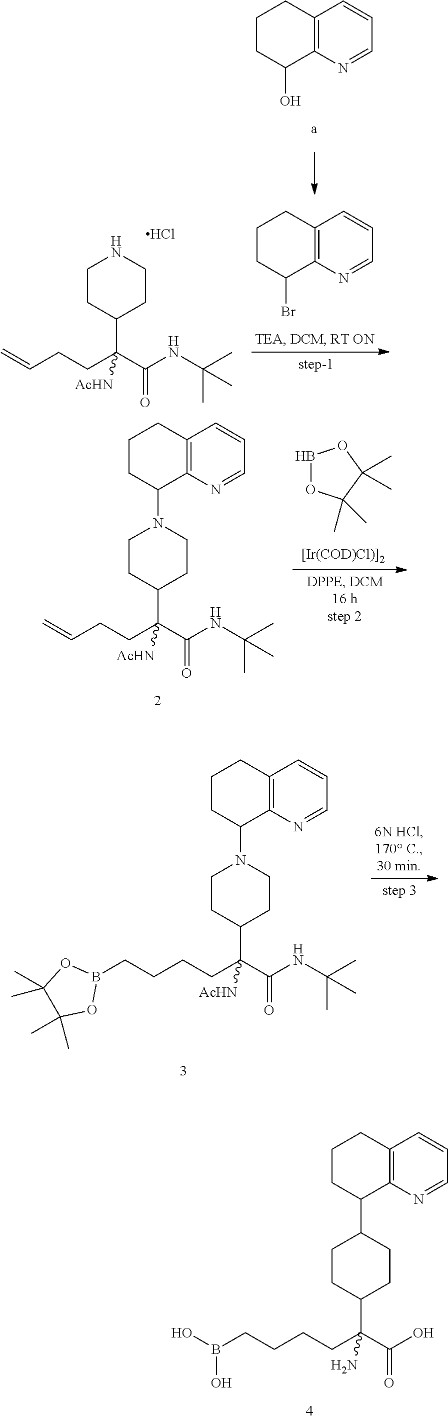

### Synthesis of 8-bromo-5,6,7,8-tetrahydroquinoline

To the stirred solution of 5,6,7,8-tetrahydroquinolin-8-ol (500 mg, 6.70 mmol) in DCM (10 mL) was added PBr₃ (0.45 mL, 8.04 mmol) drop wise at 0° C. under nitrogen atmosphere and the reaction was stirred for 3 h at RT. After the completion of reaction (TLC monitoring) the reaction mixture was evaporated under reduced pressure to get crude residue, the crude was then diluted with water (100 mL) and basified with K₂CO₃ (until basic) and then extracted with ethyl acetate (100 mL) the organic layer was separated, dried over Na₂SO₄, filtered and evaporated under reduced pressure to get crude residue which was used as such in next step without further purification.

¹H NMR: (400 MHz, DMSO-d₆) ppm 8.39 (d, J=4 Hz, 1H) 7.53 (d, J=8 Hz, 1H) 7.25-7.22 (m, 1H), 5.61 (s, 1H), 2.92-2.80 (m, 2H), 2.38-2.19 (m, 1H), 2.12-1.99 (m, 1H) 1.93-1.81 (m, 2H).

### Synthesis of 2-acetamido-N-tert-butyl-2-(1-(5,6,7,8-tetrahydroquinolin-8-yl)piperidin-4-yl)hex-5-enamide Isomer A

To the stirred solution of 8-bromo-5,6,7,8-tetrahydroquinoline (300 mg, 0.86 mmol) in DCM (10 mL) was added TEA (0.5 mL, 2.58 mmol) and the reaction mixture was stirred for 10 minutes at RT then added 2-acetamido-N-tert-butyl-2-(1-(5,6,7,8-tetrahydroquinolin-8-yl)piperidin-4-yl)hex-5-enamide Isomer A (220 mg, 1.03 mmol) to the reaction mixture. After the completion of reaction (LCMS monitoring) the reaction mass was diluted with water (150 mL) and extracted with ethyl acetate (150 mL). The organic layer was separated, dried over anhydrous Na₂SO₄, filtered and evaporated under reduce pressure to get crude residue (1 g as crude) which was used as such in next step without any further purification. LCMS: 441.4 [M+H]⁺

### Synthesis of 2-acetamido-N-tert-butyl-2-(1-(5,6,7,8-tetrahydroquinolin-8-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(5,6,7,8-tetrahydroquinolin-8-yl)piperidin-4-yl)hex-5-enamide Isomer A (990 mg as crude, 2.25 mmol) in DCM (10 mL) was added [Ir(COD)Cl]₂ (90 mg, 0.135 mmol) and DPPE (107 mg, 0.12 mmol) at RT under nitrogen atmosphere and the reaction mass was stirred for 10 minutes, after 10 minutes of stirring at RT the reaction mass was cooled to 0° C. then added 4,4,5,5-tetramethyl-1,3,2-dioxaborolane (0.40 mL, 2.70 mmol) drop wise to the reaction mixture. After the completion of reaction mixture (LCMS monitoring) the reaction mixture was diluted with water (250 mL) and extracted with DCM (250 mL). The organic layer was separated, dried over Na₂SO₄, filtered and evaporated under reduced pressure to get crude residue. The crude was taken as such for next step without any further purification. LCMS: 569.5 [M+H]⁺

### Synthesis of 2-amino-6-borono-2-(1-(5,6,7,8-tetrahydroquinolin-8-yl)piperidin-4-yl)hexanoic acid Isomer A

A stirred solution of 2-acetamido-N-tert-butyl-2-(1-(5,6,7,8-tetrahydroquinolin-8-yl)piperidin-4-yl)-6-(4,4,5,5-tetramethyl-1,3,2-dioxaborolan-2-yl)hexanamide Isomer A (1.5 g as crude, 2.78 mmol) in 6 M HCl (3 mL) was stirred in microwave for 30 minutes at 170° C. After the completion of reaction (LCMS monitoring) the reaction mixture was washed with DCM (50 mL) and the aqueous layer was lyophilized to get crude compound which was purified through reverse phase HPLC to get compound TFA salt as an off white solid (35 mg). LCMS: 372.4 [M+H−H₂O]⁺, 354.4 [M+H−2H₂O]⁺

¹H NMR: (400 MHz, D₂O) δ 8.44 (d, J=4 Hz, 1H), 7.67 (d, J=8 Hz, 1H), 7.33 (dd, J=8.4 Hz, 1H), 3.66 (t, J=8 Hz, 1H), 3.49-3.38 (m, 1H), 3.10 (t, J=12.0 Hz, 2H), 2.9-2.83 (m, 2H), 2.43-2.4 (m, 1H), 2.27-1.98 (m, 4H), 1.93-1.89 (m, 1H), 1.89-1.77 (m, 4H), 1.65-1.52 (m, 1H), 1.48-1.38 (m, 3H), 1.38-1.29 (m, 1H), 1.28-1.114 (m, 1H), 0.8 (t, J=8 Hz, 2H).

### Example A18: Synthesis of Compound 35 (Isomer A)

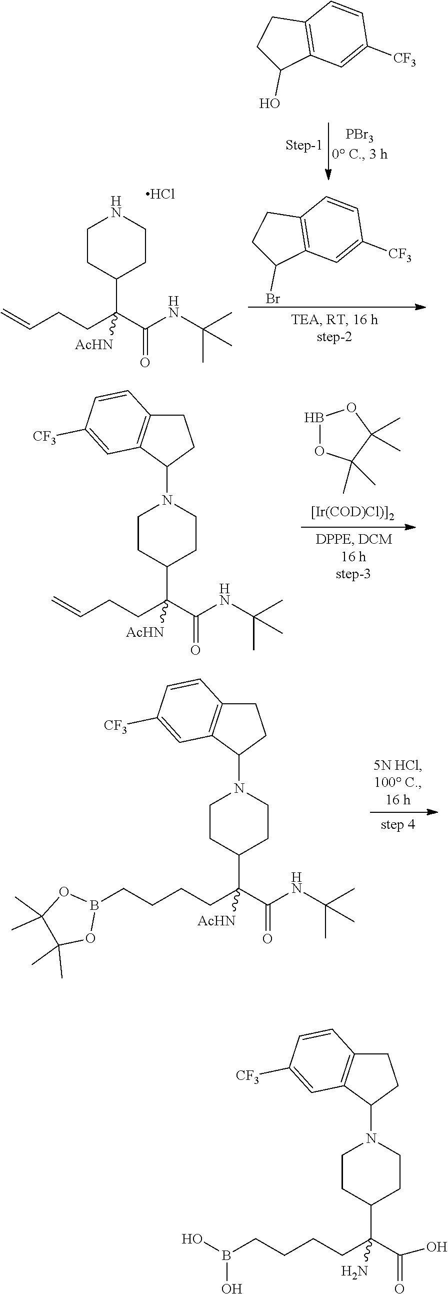

### Step-1: Synthesis of 1-bromo-6-(trifluoromethyl) indane

To a solution of 6-(trifluoromethyl)indan-1-ol (300 mg, 1.48 mmol, 1.0 eq.) in DCM (10 mL) was added phosphorous tribromide (0.24 mL, 1.93 mmol, 1.3 eq.) in DCM (3 mL) drop wise at 0° C. After completion of addition the mixture was allowed to stir at RT for 3 h. Product formation was confirmed by NMR. After completion of reaction the reaction mixture was quenched by 5% solution of Na₂CO₃ in water and extracted with DCM (50 mL×3). Combined organic layer was dried over anhydrous Na₂SO₄ and concentrated under reduced pressure to obtain 1-bromo-6-(trifluoromethyl) indane (250 mg, crude) which was used as such for next step without any further purification.

¹H NMR: (400 MHz, DMSO-d₆) δ ppm 7.68 (s, 1H), 7.52 (d, J=8.0 Hz, 1H), 7.38 (d, J=8 Hz, 1H), 5.04 (dd, J=4, 2 Hz, 1H), 3.19 (p, J=8 Hz, 1H), 2.98-2.89 (m, 1H), 2.72-2.51 (m, 2H).

### Step-2: Synthesis of 2-acetamido-N-tert-butyl-2-[1-[6-(trifluoromethyl)indan-1-yl]-4-piperidyl]hex-5-enamide Isomer A

To a stirred solution of 2-acetamido-N-tert-butyl-2-(4-piperidyl)hex-5-enamide hydrochloride Isomer A (150 mg, 0.43 mmol, 1.0 eq.) in DCM (5 mL) was added triethylamine (0.3 mL, 1.72 mmol, 2 eq.) at 0° C. The reaction mixture was allowed to stir at RT for 10 min. 1-Bromo-6-(trifluoromethyl) indane (250 mg, 0.903 mmol, 2 eq.) was added and the reaction mixture was stirred at RT for 16 h. Reaction progress was monitored by LCMS. Reaction was stopped by adding water and extracted with DCM (30 mL×3). Combined organic layer was dried over anhydrous Na₂SO₄ and concentrated under reduced pressure to obtain 2-acetamido-N-tert-butyl-2-[1-[5-(trifluoromethyl)indan-1-yl]-4-piperidyl]hex-5-enamide Isomer A (150 mg as a crude) which was directly used for next step without any further purification. LCMS: 494.4 [M+H]⁺

### Step-3: Synthesis of 2-acetamido-N-tert-butyl-6-(4,4,5,5-tetramethyl-1,3,2-dioxaborolan-2-yl)-2-[1-[6-(trifluoromethyl)indan-1-yl]-4-piperidyl]hexanamide Isomer A

To a stirred solution of 2-acetamido-N-tert-butyl-2-[1-[5-(trifluoromethyl)indan-1-yl]-4-piperidyl]hex-5-enamide Isomer A (200 mg as crude) in DCM (5 mL) was added [Ir(COD)Cl]₂ (18 mg, 0.024 mmol, 0.06 eq.) and DPPE (21 mg, 0.048 mmol, 0.012 eq.). The reaction mixture was allowed to stir at RT for 15 min then cooled to 0° C. 4,4,5,5-tetramethyl-1,3,2-dioxaborolane (0.12 mL, 0.81 mmol, 2 eq.) was added drop wise at 0° C. After completion of addition the mixture was allowed to stir at RT for overnight. Reaction progress was monitored by TLC and NMR. The reaction mixture was diluted with ethyl acetate (30 mL) and washed with brine (40 mL). Organic layer was separated and dried over anhydrous Na₂SO₄ and concentrated under reduced pressure to get 2-acetamido-N-tert-butyl-6-(4,4,5,5-tetramethyl-1,3,2-dioxaborolan-2-yl)-2-[1-[6-(trifluoromethyl)indan-1-yl]-4-piperidyl]hexanamide Isomer A (190 mg, as crude) which was taken as such for next step without any further purification. LCMS: 622.5 [M+H]⁺

### Step-4: Synthesis of 2-amino-6-borono-2-[1-[6-(trifluoromethyl)indan-1-yl]-4-piperidyl]hexanoic acid Isomer A

A stirred solution of 2-acetamido-N-tert-butyl-6-(4,4,5,5-tetramethyl-1,3,2-dioxaborolan-2-yl)-2-[1-[6-(trifluoromethyl)indan-1-yl]-4-piperidyl]hexanamide Isomer A (190 mg, crude) from previous step was dissolved in 4 M HCl/Dioxane (5 mL) and the mixture was heated at 100° C. for 16 h. Product formation was confirmed by LCMS. The mixture was concentrated under reduced pressure and then diluted with water (25 mL) and washed with DCM (15 mL×2). Aqueous phase was separated and freeze dried over lyophilizer. The crude product was purified by reversed phase HPLC to obtain 2-amino-6-borono-2-[1-[6-(trifluoromethyl)indan-1-yl]-4-piperidyl]hexanoic acid Isomer A (43.5 mg) as an off-white solid (formate salt).

¹H NMR: (400 MHz, D₂O) δ8.45 (s, 1H, formate), 7.90 (s, 1H), 7.80 (d, J=8.0 Hz, 1H), 7.61 (d, J=8.0 Hz, 1H), 5.02 (d, J=8.0 Hz, 1H), 3.62 (t, J=12 Hz, 1H), 3.43 (t, J=12 Hz, 1H), 3.3-2.96 (m, 4H), 2.69-2.46 (m, 2H), 2.21-2.04 (m, 2H), 1.96-1.77 (m, 4H), 1.71-1.39 (m, 4H), 1.28-1.12 (m, 1H), 0.79 (t, J=8 Hz, 2H).

### Example A19: Synthesis of Compound 48

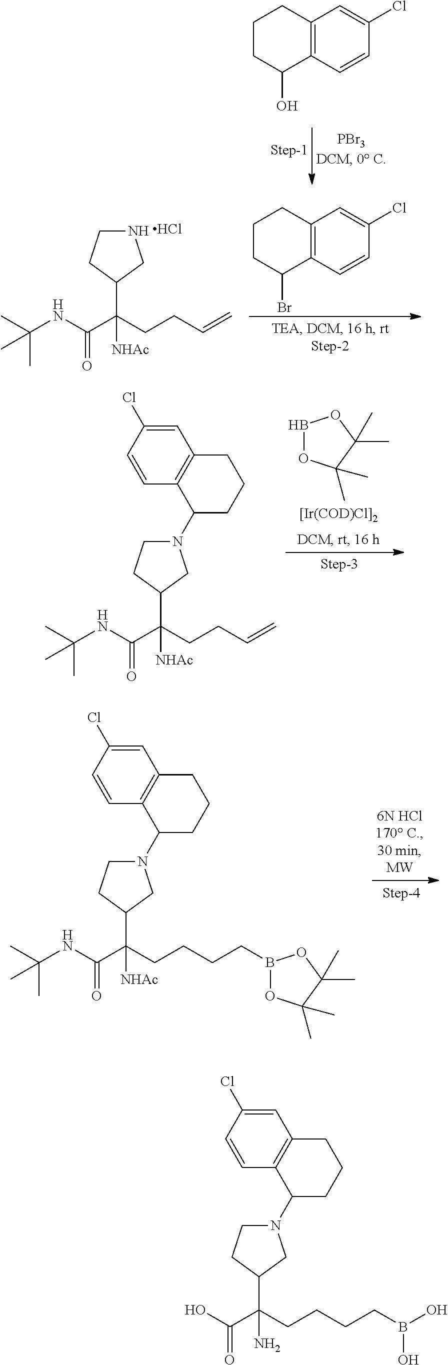

### Step-1: Synthesis of 1-bromo-6-chloro-tetralin

To a solution of 6-chlorotetralin-1-ol (800 mg, 4.39 mmol, 1.0 eq.) in DCM (6 mL) was added phosphorous tribromide (0.543 mL, 5.71 mmol, 1.3 eq.) in DCM (5 mL) drop wise at 0° C. After completion of addition the mixture was allowed to stir at RT for 3 h. Product formation was confirmed by NMR. After completion of reaction the reaction mixture was quenched by using 5% solution of Na₂CO₃ in water and extracted with DCM (50 mL×3). Combined organic layer was dried over anhydrous Na₂SO₄ and concentrated under reduced pressure to obtain 1-bromo-6-chloro-tetralin (600 mg) which was used as such in next step without any further purification.

¹H NMR: (400 MHz, CHLOROFORM-d) δ ppm 7.27 (d, J=8 Hz, 1H), 7.11 (dd, J=8, 4 Hz, 1H), 7.07 (br.s., 1H), 5.53 (t, J=4 Hz, 1H), 2.96-2.74 (m, 2H), 2.44-2.34 (d, J=16 Hz, 1H), 2.30-2.03 (m, 2H), 1.95-1.84 (m, 1H)

### Step-2: Synthesis of 2-acetamido-2-[1-(6-chlorotetralin-1-yl)pyrrolidin-3-yl]-N-isopropyl-hex-5-enamide

To a stirred solution of 2-acetamido-N-tert-butyl-2-pyrrolidin-3-yl-hex-5-enamide hydrochloride (500 mg, 1.51 mmol, 1.0 eq) in DCM (5 mL) was added triethylamine (412 mg, 1.81 mmol, 1.2 eq) at 0° C. The reaction mixture was allowed to stir at RT for 10 min. 1-bromo-6-chloro-tetralin (592 mg, 3.02 mmol, 2.0 eq.) was added and the reaction mixture was stirred at RT for 16 h. The progress of the reaction was monitored by LCMS. The reaction was quenched by addition of water and extracted with DCM (40 mL×3). Combined organic layer was dried over anhydrous Na₂SO₄ and concentrated under reduced pressure to obtain 2-acetamido-2-[1-(6-chlorotetralin-1-yl)pyrrolidin-3-yl]-N-isopropyl-hex-5-enamide (400 mg as a crude) which was directly used for next step. LCMS: 460.5 [M]⁺

### Step-3: Synthesis of 2-acetamido-2-[1-(6-chlorotetralin-1-yl)pyrrolidin-3-yl]-N-isopropyl-6-(4,4,5,5-tetramethyl-1,3,2-dioxaborolan-2-yl)hexanamide

To a stirred solution of 2-acetamido-2-[1-(6-chlorotetralin-1-yl)pyrrolidin-3-yl]-N-isopropyl-hex-5-enamide (400 mg, 0.86 mmol, 1.0 eq.) in DCM (15 mL) was added [Ir(COD)Cl]₂ (34 mg, 0.018 mmol, 0.06 eq.) and DPPE (10 mg, 0.025 mmol, 0.03 eq). The reaction mixture was allowed to stir at RT for 15 min then cooled to 0° C. 4,4,5,5-tetramethyl-1,3,2-dioxaborolane (0.7 mL, 4.76 mmol, 1.5 eq.) was added drop wise at 0° C. After completion of addition the mixture was allowed to stir at RT for overnight. Reaction progress was monitored by TLC and NMR. After completion of reaction, the reaction mixture was diluted with ethyl acetate (100 mL) and washed with brine (50 mL). Organic layer was separated and dried over anhydrous Na₂SO₄ and concentrated under reduced pressure to yield 2-acetamido-2-[1-(6-chlorotetralin-1-yl)pyrrolidin-3-yl]-N-isopropyl-6-(4,4,5,5-tetramethyl-1,3,2-dioxaborolan-2-yl)hexanamide (300 mg, as crude) which was used as such in next step. LCMS: 588.6 [M]⁺

### Step-4: Synthesis of 2-amino-6-borono-2-[1-(6-chlorotetralin-1-yl)pyrrolidin-3-yl]hexanoic acid

To a stirred solution of 2-acetamido-2-[1-(6-chlorotetralin-1-yl)pyrrolidin-3-yl]-N-isopropyl-6-(4,4,5,5-tetramethyl-1,3,2-dioxaborolan-2-yl)hexanamide (300 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (10 mL×2). Aqueous phase was separated and freeze dried using a lyophilizer. The crude product was purified by reverse phase HPLC to obtain 2-amino-6-borono-2-[1-(6-chlorotetralin-1-yl)pyrrolidin-3-yl]hexanoic acid TFA salt (60 mg) as an off-white solid. LCMS: 409.1 [M+H]⁺ & 391.1[M+H−H₂O]⁺

¹H NMR: (400 MHz, D₂O) δ 7.42-7.23 (m, 3H), 4.48 (br.s., 1H), 3.85-3.63 (m, 1H), 3.62-3.38 (m, 2H), 3.04-2.71 (m, 3H), 2.44-2.24 (m, 2H), 2.21-1.59 (m, 7H), 1.38 (br.s., 3H), 1.30-1.1 (m, 1H), 0.76 (t, J=8 Hz, 2H).

### Example A20: Synthesis of Compound 77

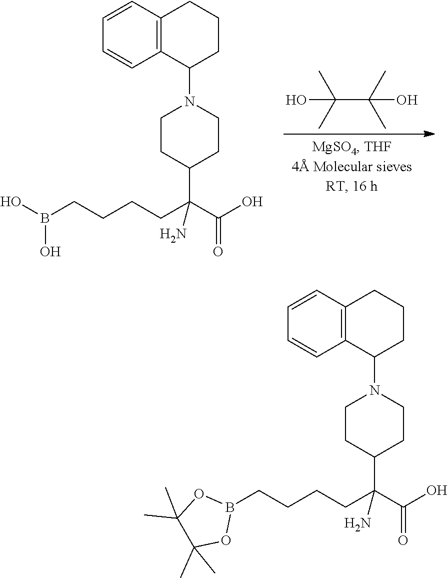

### Step-1: 2-amino-2-(1-(1,2,3,4-tetrahydronaphthalen-1-yl)piperidin-4-yl)-6-(4,4,5,5-tetramethyl-1,3,2-dioxaborolan-2-yl)hexanoic acid

To a stirred solution of 2-amino-6-borono-2-(1-(1,2,3,4-tetrahydronaphthalen-1-yl)piperidin-4-yl)hexanoic acid (100 mg, 0.25 mmol) in dry THF (5 mL) was added pinacol (30 mg, 0.25 mmol) followed by addition of MgSO₄ (34 mg, 0.28 mmol) and 4 Å molecular sieves (5 mg). The resultant reaction mixture was stirred at room temperature for 16 h. After that, solvent was evaporated in vacuo and the crude was purified by reversed phase HPLC to obtain 2-amino-2-(1-(1,2,3,4-tetrahydronaphthalen-1-yl)piperidin-4-yl)-6-(4,4,5,5-tetramethyl-1,3,2-dioxaborolan-2-yl)hexanoic acid (4 mg) as a white solid.

¹H NMR (400 MHz, D₂O) δ: 7.43-7.36 (m, 2H), 7.29 (t, J=8 Hz, 2H), 3.59 (d, J=12 Hz, 1H), 3.47 (d, J=12 Hz, 1H), 3.19-3.05 (m, 2H), 2.88-2.76 (m, 2H), 2.26-2.02 (m, 3H), 1.85-1.69 (m, 6H), 1.58-1.28 (m, 4H), 1.24 (s, 12H), 1.22-1.15 (m, 3H), 0.78 (t, J=8 Hz, 2H).

### Example A21: Synthesis of Compound 79 (Isomer A)

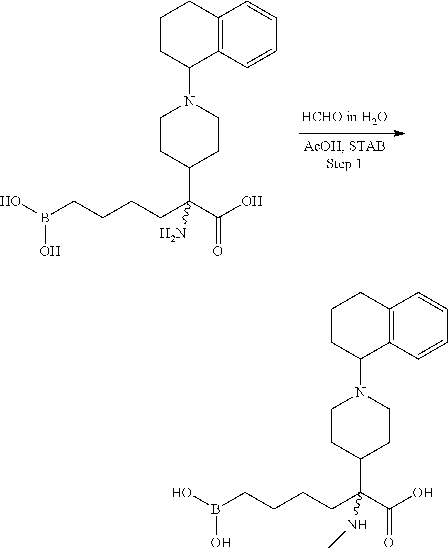 [SMILES: NC(CCCCB(O)O)(C(=O)O)C1CCN(C2CCCc3ccccc32)CC1 ; CNC(CCCCB(O)O)(C(=O)O)C1CCN(C2CCCc3ccccc32)CC1]

To the stirred solution of 2-amino-6-borono-2-(1-(1,2,3,4-tetrahydronaphthalen-1-yl)piperidin-4-yl)hexanoic acid Isomer A (50 mg, 1.34 mmol) in 37% HCHO in water solution (0.5 mL) was added catalytic amount of AcOH (1-2 drops) and the reaction mixture was stirred for 1.5 h at RT under nitrogen atmosphere, then added sodium triacetoxyborohydride (25 mg, 1.34 mmol) and reaction was continued for 16 h at RT. After the completion of reaction the crude was lyophilized and purified by reverse phase chromatography to yield 6-borono-2-(methylamino)-2-(1-(1,2,3,4-tetrahydronaphthalen-1-yl)piperidin-4-yl)hexanoic acid TFA salt Isomer A as a white solid (15 mg). LCMS: 385.2 [M+H−H₂O]⁺.

¹H NMR: (400 MHz, D₂O) δ 7.44 (d, J=7.6 Hz, 1H), 7.37-7.33 (m, 1H), 7.27 (t, J=8 Hz, 2H), 3.37 (m, 1H), 3.28-3.14 (m, 1H), 2.87-2.73 (m, 4H), 2.59 (s, 2H), 2.15-2.06 (m, 2H), 2.04-1.91 (m, 4H), 1.89 (s, 3H), 1.78-1.69 (m, 2H), 1.6-1.55 (m, 1H) 1.48-1.35 (m, 3H), 1.33-1.27 (m, 1H), 1.19-1.07 (m, 1H), 0.78 (t, J=8 Hz, 2H).

### Example A22: Synthesis of Compound 81

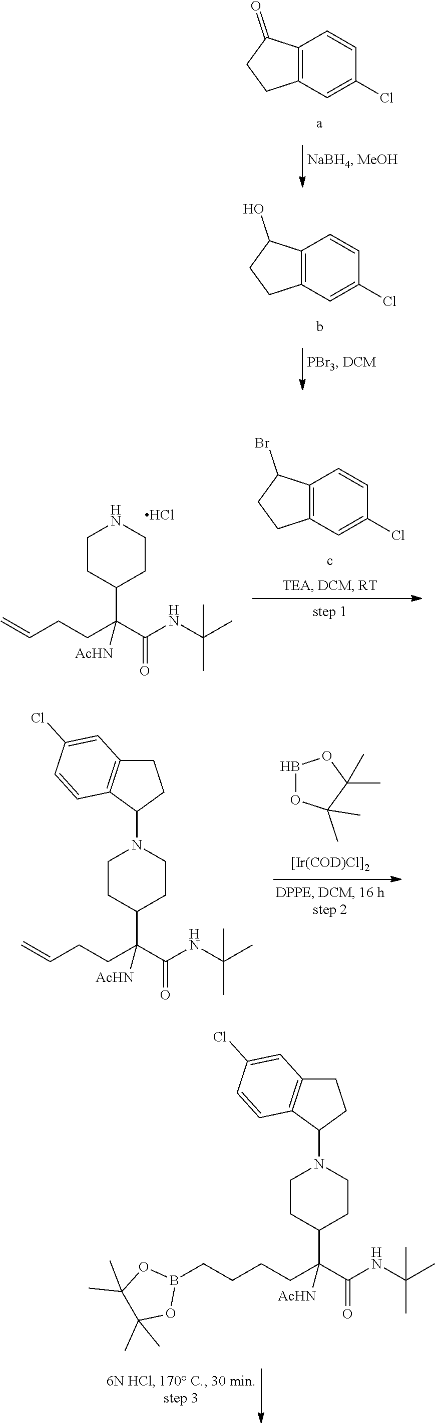

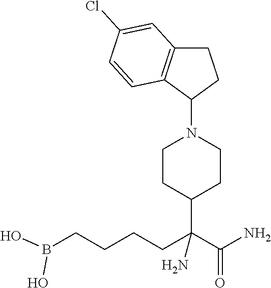 [SMILES: NC(=O)C(N)(CCCCB(O)O)C1CCN(C2CCc3cc(Cl)ccc32)CC1]

### Synthesis of intermediate b (5-chloro-2,3-dihydro-1H-inden-1-one)

To the stirred solution of 5-chloro-indanone (2 g, 12.03 mmol) in methanol (10 mL) was added NaBH₄ (683 mg, 18.05 mmol) portion wise at 0° C. under nitrogen atmosphere. The reaction mixture was stirred for 1 h at RT under nitrogen atmosphere. After the completion of reaction (TLC monitoring) the reaction mass was evaporated under reduced pressure to give crude reaction mixture. The crude reaction mixture was then further diluted with water (150 mL) and extracted with ethyl acetate (150 mL). The organic layer was separated, dried over anhydrous sodium sulfate, filtered and evaporated to give desired product. LCMS: 169.2 [M+H]⁺.

### Synthesis of Intermediate c (1-bromo-5-chloro-2,3-dihydro-1H-indene)

To the stirred solution of 5-chloro-2,3-dihydro-1H-inden-1-one (2 g, 11.9 mmol) in DCM (10 mL) was added PBr₃ (1.35 mL, 14.28 mmol) drop wise at 0° C. under nitrogen atmosphere. The reaction mixture was stirred at RT for 3 h under same conditions. After the completion of reaction (TLC monitoring) the reaction mass was evaporated under reduced pressure to give crude, the crude was then diluted with water (100 mL) and extracted with ethyl acetate (100 mL) the organic layer was separated, dried over anhydrous sodium sulphate, filtered and evaporated under reduced pressure. LCMS: 231.2 [M]⁺.

### Example A23: Synthesis of Compound 82

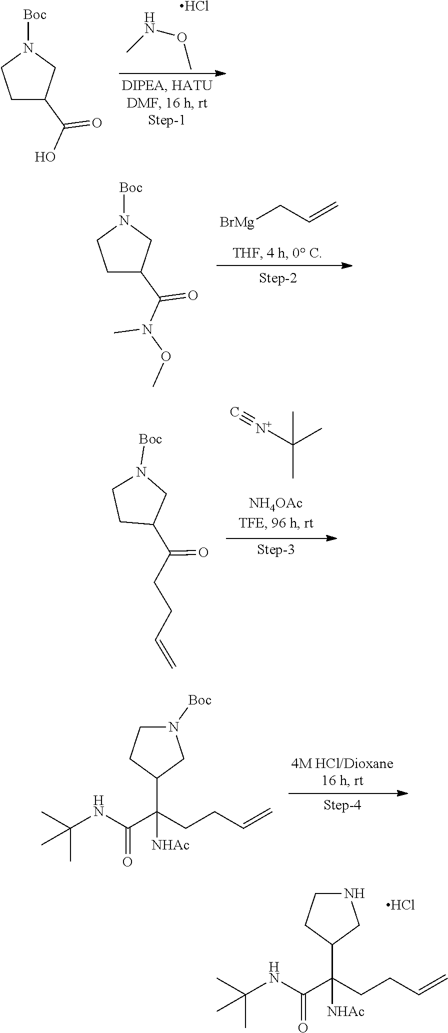

### Step-1: Synthesis of tert-butyl 3-[methoxy(methyl)carbamoyl]pyrrolidine-1-carboxylate

To the stirred solution of 1-tert-butoxycarbonylpyrrolidine-3-carboxylic acid (10 g, 46.51 mmol, 1 eq) in DMF (30 mL) was added HATU (26.5 g, 67.71 mmol, 1.5 eq) and DIPEA (17 mL, 93.13 mmol, 2 eq) resulted reaction mixture was allowed to stir for 15 min followed by addition of N,O-Dimethylhydroxylamine hydrochloride (5.1 g, 55.81 mmol, 1.2 eq). The reaction mixture was allowed to stir at RT for 16 h. Product formation was monitored by TLC and LCMS. After completion of reaction the reaction mixture was diluted with water (500 mL) and extracted with ethyl acetate (500 mL×3). Combined organic layer was washed with brine (100 mL), dried over anhydrous Na₂SO₄ and concentrated under reduced pressure. The crude product thus obtained was purified by flash chromatography (0-40% ethyl acetate in hexane as an eluent) to obtain tert-butyl 3-[methoxy(methyl)carbamoyl]pyrrolidine-1-carboxylate (7.5 g) as a transparent oil. This reaction was performed in 2 batches of 10 g each resulting in a total yield of (7.5 g+7.6 g=15.1 g). LCMS: 259.0 [M+F1]⁺.

### Step-2: Synthesis of tert-butyl 3-pent-4-enoylpyrrolidine-1-carboxylate

To the stirred solution of tert-butyl 3-[methoxy(methyl)carbamoyl]pyrrolidine-1-carboxylate (10 g, 38.75 mmol, 1 eq) in THF (100 ml) was added but-3-enylmagnesium bromide (40 mL, 38.75 mmol). The reaction mixture was allowed to stir at RT for 4 h. Product formation was monitored by TLC and LCMS. After completion of reaction the reaction mixture was quenched by 1 N citric acid (100 mL) and extracted with ethyl acetate (250 mL×3). Combined organic layer was washed with brine (100 mL), dried over anhydrous Na₂SO₄ and concentrated under reduced pressure. The crude product thus obtained was purified by flash chromatography (0-40% ethyl acetate in hexane as an eluent) to obtain tert-butyl 3-pent-4-enoylpyrrolidine-1-carboxylate (9.0 g) as a transparent oil. LCMS: 254.0 [M+H]⁺.

### Step-3: Synthesis of tert-butyl 3-[1-acetamido-1-(tert-butylcarbamoyl)pent-4-enyl]pyrrolidine-1-carboxylate

To a solution of tert-butyl 3-pent-4-enoylpyrrolidine-1-carboxylate (9.0 g, 31.5 mmol 1 eq) in trifluoroethanol (15 mL) was added ammonium acetate (3.39 g, 126.0 mmol, 4 eq) and tert-Butyl isocyanide (7.3 mL, 63.0 mmol, 2 eq). The resultant reaction mixture was stirred for 4 days at room temperature. The reaction progress was monitored by TLC & LCMS. After completion of reaction, the mixture was concentrated under reduced pressure to afford the crude reaction mixture. The crude was diluted with water (150 mL) and extracted using ethyl acetate (300×3 mL). Organic layer was separated and dried over anhydrous Na₂SO₄ and concentrated under reduced pressure. The crude product was purified by flash chromatography (0-50% ethyl acetate in hexane as an eluent) to obtain of tert-butyl 3-[1-acetamido-1-(tert-butylcarbamoyl)pent-4-enyl]pyrrolidine-1-carboxylate (8.5 g) as an off-white solid. LCMS: 396.3 [M+H]⁺.

### Step-4: Synthesis of 2-acetamido-N-tert-butyl-2-pyrrolidin-3-yl-hex-5-enamide hydrochloride

To a stirred solution tert-butyl 3-[1-acetamido-1-(tert-butylcarbamoyl)pent-4-enyl]pyrrolidine-1-carboxylate (7 g, 17.67 mmol) in DCM (5 mL) was added 4 N HCl in dioxane (10 mL). The reaction mixture was allowed to stir at RT overnight. The product formation was confirmed by ¹H NMR. The reaction mixture was concentrated under reduced pressure to obtain 2-acetamido-N-tert-butyl-2-pyrrolidin-3-yl-hex-5-enamide hydrochloride (5.5 g). LCMS: 296.1 [M+H]⁺

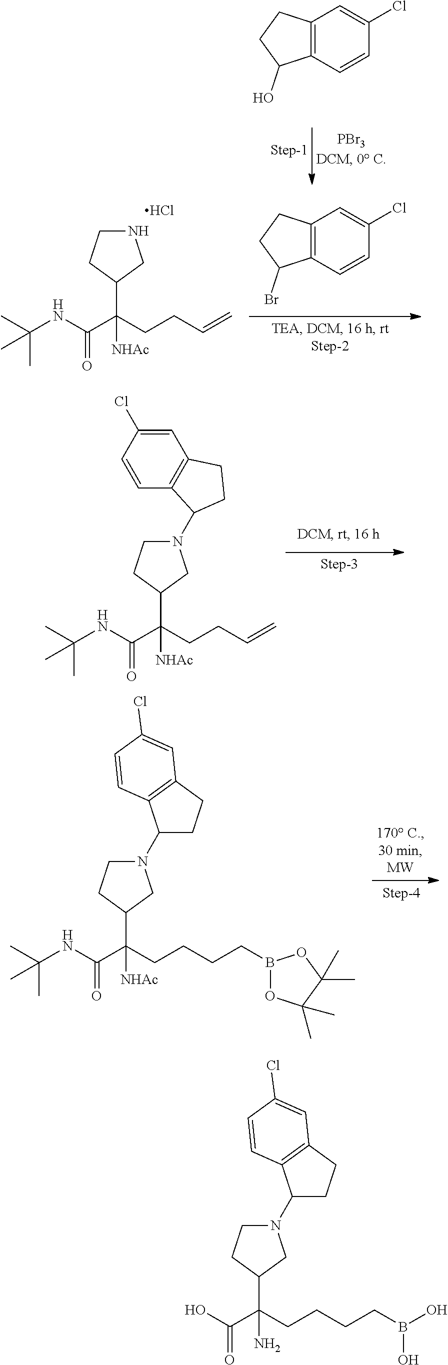

### Step 1: Synthesis of 1-bromo-5-chloro-indane

To a solution of 5-chloroindan-1-ol (1000 mg, 5.95 mmol, 1.0 eq.) in DCM (7 mL) was added phosphorous tribromide (0.8 mL, 7.73 mmol, 1.3 eq.) in DCM (3 mL) drop wise at 0° C. After completion of addition the mixture was allowed to stir at RT for 3 h. The product formation was confirmed by NMR. After completion of reaction the reaction mixture was quenched by 5% solution of Na₂CO₃ in water and extracted with DCM (50 mL×3). Combined organic layer was dried over anhydrous Na₂SO₄ and concentrated under reduced pressure to obtain 1-bromo-5-chloro-indane (800 mg) as an oil.

¹H NMR: (400 MHz, CHLOROFORM-d) δ ppm 7.36-7.29 (m, 1H), 7.25-7.16 (m, 2H), 5.51 (dd, J=8, 2 Hz, 1H), 3.23-3.12 (m, 1H), 2.92-2.75 (m 1H), 2.67-2.46 (m, 2H).

### Step-2: Synthesis of 2-acetamido-N-tert-butyl-2-[1-(5-chloroindan-1-yl)pyrrolidin-3-yl]hex-5-enamide

To a stirred solution of 2-acetamido-N-tert-butyl-2-pyrrolidin-3-yl-hex-5-enamide hydrochloride (500 mg, 1.51 mmol, 1.0 eq.) in DCM (5 mL) was added triethylamine (412 mg, 1.81 mmol, 1.2 eq.) at 0° C. The reaction mixture was allowed to stir at RT for 10 min and then 1-bromo-5-chloro-indane (418 mg, 3.02 mmol, 2.0 eq.) was added and the reaction mixture was stirred at RT for 16 h. The reaction progress was monitored by LCMS. After completion of reaction it was quenched by addition of water and extracted with DCM (40 mL×3). Combined organic layer was dried over anhydrous Na₂SO₄ and concentrated under reduced pressure to obtain 2-acetamido-N-tert-butyl-2-[1-(5-chloroindan-1-yl)pyrrolidin-3-yl]hex-5-enamide (350 mg as a crude) which was directly used for next step without any further purification. LCMS: 446.2 [M]⁺

### Step-3: Synthesis of 2-acetamido-N-tert-butyl-2-[1-(5-chloroindan-1-yl)pyrrolidin-3-yl]-6-(4,4,5,5-tetramethyl-1,3,2-dioxaborolan-2-yl)hexanamide

To a stirred solution of 2-acetamido-N-tert-butyl-2-[1-(5-chloroindan-1-yl)pyrrolidin-3-yl]hex-5-enamide (350 mg, 0.67 mmol, 1.0 eq.) in DCM (15 mL) was added [Ir(COD)Cl]₂ (30 mg, 0.018 mmol, 0.03 eq.) and DPPE (10 mg, 0.03 mmol, 0.03 eq.). The reaction mixture was allowed to stir at RT for 15 min then cooled to 0° C. 4,4,5,5-tetramethyl-1,3,2-dioxaborolane (0.7 mL, 4.76 mmol, 1.5 eq.) was added drop wise at 0° C. After completion of addition the mixture was allowed to stir at RT for overnight. Reaction progress was monitored by TLC and NMR. The reaction mixture was diluted with ethyl acetate (100 mL) and washed with brine (50 mL). Organic layer was separated and dried over anhydrous Na₂SO₄ and concentrated under reduced pressure to get 2-acetamido-N-tert-butyl-2-[1-(5-chloroindan-1-yl)pyrrolidin-3-yl]-6-(4,4,5,5-tetramethyl-1,3,2-dioxaborolan-2-yl)hexanamide (300 mg, as crude) which was used as such in next step without any further purification. LCMS: 574.4 [M]⁺

### Step-4: Synthesis of 2-amino-6-borono-2-[1-(5-chloroindan-1-yl)pyrrolidin-3-yl]hexanoic acid

To a stirred solution of 2-acetamido-N-tert-butyl-2-[1-(5-chloroindan-1-yl)pyrrolidin-3-yl]-6-(4,4,5,5-tetramethyl-1,3,2-dioxaborolan-2-yl)hexanamide (300 mg as a crude) from previous step was dissolved in 6 N HCl and the mixture was heated at 170° C. in microwave reactor for 30 min. Product formation was confirmed by LCMS. The mixture was then diluted with water (25 mL) and washed with DCM (10 mL×2). Aqueous phase was separated and lyophilized. The crude product was purified by reverse phase HPLC to obtain 2-amino-6-borono-2-[1-(5-chloroindan-1-yl) pyrrolidin-3-yl]hexanoic acid TFA salt (10 mg) as an off-white solid. LCMS: 377.1 [M+H−H₂O]⁺

¹H NMR: (400 MHz, D₂O) δ 7.55-7.37 (m, 2H), 7.36-7.25 (m, 1H), 3.7-3.5 (m, 1H), 3.49-3.25 (m, 3H), 3.2-3.04 (m, 1H), 3.03-2.89 (m, 1H), 2.87-2.69 (m, 1H), 2.62-2.42 (m, 1H), 2.41-2.15 (m, 1H), 2.15-1.98 (m, 1H), 1.96-1.57 (m, 4H), 1.54-1.22 (m, 3H), 1.22-1.07 (m, 1H), 0.85-0.59 (m, 2H).

### Example A24: Synthesis of Compound 83 (Isomer A)

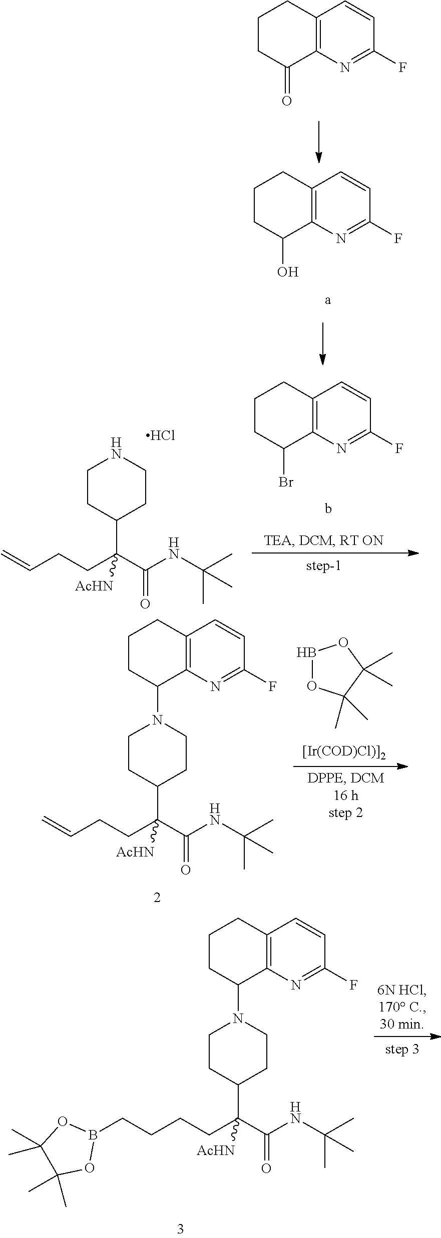

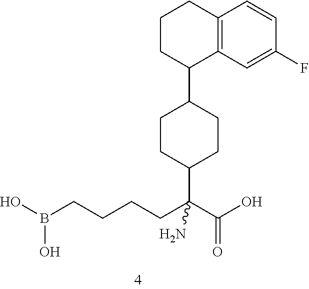 [SMILES: NC(CCCCB(O)O)(C(=O)O)C1CCC(C2CCCc3ccc(F)cc32)CC1]

### Synthesis of 7-fluoro-1,2,3,4-tetrahydronaphthalen-1-ol

To the stirred solution of 7-fluoro-3,4-dihydronaphthalen-1(2H)-one (500 mg, 3.04 mmol) in methanol (10 mL) was added NaBH₄ (180 mg, 4.57 mmol) portion wise at 0° C. under nitrogen atmosphere. After the completion of reaction (TLC monitoring) the reaction mixture was evaporated under reduced pressure to get crude residue. The crude thus obtained was then diluted with water (100 mL) and extracted with ethyl acetate (100 mL). The organic layer was separated, dried over Na₂SO₄, filtered and evaporated under reduced pressure to get crude product as 7-fluoro-1,2,3,4-tetrahydronaphthalen-1-ol.

¹H NMR: (400 MHz, CHLOROFORM-d) δ ppm 7.16 (dd, J=9.65, 3.07 Hz, 1H) 6.98-7.09 (m, 1H) 6.89 (td, J=8.44, 2.85 Hz, 1H) 4.74 (d, J=4.39 Hz, 1H) 2.73-2.86 (m, 1H) 2.59-2.73 (m, 1H) 1.90-2.10 (m, 2H) 1.69-1.90 (m, 3H).

### Synthesis of 1-bromo-7-fluoro-1,2,3,4-tetrahydronaphthalene

To the stirred solution of 7-fluoro-1,2,3,4-tetrahydronaphthalen-1-ol (500 mg, 3.01 mmol) in DCM (10 mL) was added PBr₃ (0.42 mL, 4.51 mmol) drop wise at 0° C. under nitrogen atmosphere and stirred for 3 h at RT. After the completion of reaction (TLC monitoring) the reaction mixture was evaporated under reduced pressure to get crude residue. The crude was then diluted with water (150 mL) and extracted with ethyl acetate (150 mL), the organic layer was separated, dried over Na₂SO₄, filtered and evaporated under reduced pressure to get crude residue as 1-bromo-7-fluoro-1,2,3,4-tetrahydronaphthalene.

¹H NMR: (400 MHz, DMSO-d₆) δ ppm 7.11-7.17 (m, 2H) 7.01-7.11 (m, 1H) 5.81 (br. s., 1H) 2.72-2.93 (m, 2H) 2.20-2.31 (m, 1H) 1.98-2.20 (m, 2H) 1.72-1.89 (m, 1H).

### Synthesis of 2-acetamido-N-tert-butyl-2-(1-(7-fluoro-1,2,3,4-tetrahydronaphthalen-1-yl)piperidin-4-yl)hex-5-enamide Isomer A

To the stirred solution of 1-bromo-7-fluoro-1,2,3,4-tetrahydronaphthalene (250 mg, 1.09 mmol) in DCM (5 mL) was added 2-acetamido-N-tert-butyl-2-(piperidin-4-yl)hex-5-enamide hydrochloride Isomer A (300 mg, 0.86 mmol) drop wise at 0° C. and the reaction mixture was stirred at RT for 3 h, after the completion of reaction (LCMS monitoring) the reaction mass was diluted with water (100 mL) and extracted with DCM (100 mL) the organic layer was separated, dried over Na₂SO₄, filtered and evaporated under reduced pressure to get crude product as 2-acetamido-N-tert-butyl-2-(1-(7-fluoro-1,2,3,4-tetrahydronaphthalen-1-yl)piperidin-4-yl)hex-5-enamide Isomer A which was used as such in next step without any further purification. LCMS: 458.3 [M+H]⁺

### Synthesis of 2-acetamido-N-(tert-butyl)-2-(1-(7-fluoro-1,2,3,4-tetrahydronaphthalen-1-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(7-fluoro-1,2,3,4-tetrahydronaphthalen-1-yl)piperidin-4-yl)hex-5-enamide Isomer A (850 mg as crude) in DCM (10 mL) was added [Ir(COD)Cl]₂ (74 mg, 0.11 mmol) and DPPE (88 mg, 0.22 mmol) and the reaction mixture was stirred for 10 minutes at RT then cooled to 0° C. then added 4,4,5,5-tetramethyl-1,3,2-dioxaborolane (0.33 mL, 2.23 mmol) and continued stirring for 16 h at RT. After the completion of reaction (LCMS monitoring) the reaction was diluted with water (100 mL) and extracted with DCM (250 mL). The organic layer was separated, dried over Na₂SO₄, filtered and evaporated under reduced pressure to get crude product as 2-acetamido-N-tert-butyl-2-(1-(7-fluoro-1,2,3,4-tetrahydronaphthalen-1-yl)piperidin-4-yl)-6-(4,4,5,5-tetramethyl-1,3,2-dioxaborolan-2-yl)hexanamide Isomer A which was used as such in next step without any further purification. LCMS: 586.5 [M+H]⁺

### Synthesis of 2-amino-6-borono-2-(1-(7-fluoro-1,2,3,4-tetrahydronaphthalen-1-yl)piperidin-4-yl)hexanoic acid Isomer A

To the stirred solution of 2-acetamido-N-tert-butyl-2-(1-(7-fluoro-1,2,3,4-tetrahydronaphthalen-1-yl)piperidin-4-yl)-6-(4,4,5,5-tetramethyl-1,3,2-dioxaborolan-2-1)hexanamide Isomer A (1.5 g as crude) in 6 M HCl (3 mL). The reaction was subjected to MW irradiation for 30 minutes at 170° C. After the completion of reaction (LCMS monitoring) the reaction mass was washed with DCM (50 mL) and the aqueous layer was lyophilized to get crude which was further purified by reverse phase chromatography to get compound 2-amino-6-borono-2-(1-(7-fluoro-1,2,3,4-tetrahydronaphthalen-1-yl)piperidin-4-yl)hexanoic acid Isomer A TFA salt as an off white solid (35 mg). LCMS: 389.3 [M+H−H₂O]⁺.

¹H NMR: (400 MHz, D₂O) δ 7.37-7.2 (m 2H), 7.15 (t, J=8.6 Hz, 1H), 3.53 (t, J=8 Hz, 1H), 3.35 (t, J=8 Hz, 1H), 3.14-3.05 (m, 1H), 3.01-2.91 (m 1H), 2.8 (s, 2H), 2.21-2.02 (m, 4H), 2.012.01-1.69 (m, 6H), 1.67-1.29 (m, 5H), 1.24-1.14 (m, 1H), 0.79 (t, J=8 Hz, 2H).

### Step 1: Synthesis of 2-acetamido-N-tert-butyl-2-(1-(5-chloro-2,3-dihydro-1H-inden-1-yl) piperidin-4-yl) hex-5-enamide

To a stirred solution of 2-acetamido-N-tert-butyl-2-(piperidin-4-yl)hex-5-enamide (1 g, 2.89 mmol, 1.0 eq.) in DCM (4 mL) was added triethylamine (0.8 mL, 5.78 mmol, 2.0 eq.) at 0° C., the reaction mixture was allowed to stir at RT for 10 min then added 1-bromo-5-chloro-2,3-dihydro-1H-indene (800 mg, 3.46 mmol, 1.2 eq.) and the reaction mixture was stirred at RT for 16 h. The reaction progress was monitored by LCMS. Reaction was quenched by diluting it with water (250 mL) and extracted with DCM (150 mL×2). The organic layer was separated, dried over anhydrous Na₂SO₄ and concentrated under reduced pressure to obtain 2-acetamido-N-tert-butyl-2-(1-(5-chloro-2,3-dihydro-1H-inden-1-yl)piperidin-4-yl)hex-5-enamide (310 mg as crude) which was directly taken for the next step without any purification. LCMS: 460.3 [M]⁺.

### Step 2: Synthesis of 2-acetamido-N-tert-butyl-2-(1-(5-chloro-2,3-dihydro-1H-inden-1-yl) piperidin-4-yl)-6-(4,4,5,5-tetramethyl-1,3,2-dioxaborolan-2-yl)hexanamide

To a stirred solution of 2-acetamido-N-tert-butyl-2-(1-(5-chloro-2,3-dihydro-1H-inden-1-yl)piperidin-4-yl)hex-5-enamide (310 mg, 0.675 mmol, 1.0 eq.) in DCM (4 mL) was added [Ir(COD)Cl]₂ (13 mg, 0.020 mmol, 0.06 eq.) and DPPE (16 mg, 0.040 mmol, 0.12 eq.) and the reaction mixture was stirred for 15 minutes at RT. Then the reaction mixture was cooled to 0° C. then added 4,4,5,5-tetramethyl-1,3,2-dioxaborolane (0.11 mL, 0.810 mmol, 1.2 eq.) then the reaction mixture was allowed to stir at RT. The reaction progress was monitored by LCMS. Reaction was quenched by diluting it with water (125 mL) and it was then extracted with DCM (125 mL×2). The organic layer was separated, dried over anhydrous Na₂SO₄ and concentrated under reduced pressure to obtain 2-acetamido-N-tert-butyl-2-(1-(5-chloro-2,3-dihydro-1H-inden-1-yl)piperidin-4-yl)-6-(4,4,5,5-tetramethyl-1,3,2-dioxaborolan-2-yl)hexanamide (600 mg as crude) which was directly taken for next step. LCMS: 588.5 [M]⁺.

### Step 3: Synthesis of 2-amino-6-borono-2-(1-(5-chloro-2,3-dihydro-1H-inden-1-yl)piperidin-4-yl)hexanoic acid

The crude from last step 2-acetamido-N-tert-butyl-2-(1-(5-chloro-2,3-dihydro-1H-inden-1-yl)piperidin-4-yl)-6-(4,4,5,5-tetramethyl-1,3,2-dioxaborolan-2-yl)hexanamide (600 mg) was dissolved in 6N HCl and subjected to microwave irradiation for 30 minutes at 170° C. The reaction was monitored through LCMS. The mixture was diluted with water (125 mL) and washed with DCM (50 mL×2). The aqueous layer was separated and lyophilized to give crude as off white solid, the crude was purified through reverse phase HPLC to obtain final product 2-amino-6-borono-2-(1-(5-chloro-2,3-dihydro-1H-inden-1-yl)piperidin-4-yl)hexanoic acid as free base (off white solid, 10 mg). LCMS: 391.1 [M−H₂O]⁺.

¹H NMR: (400 MHz, D₂O) δ 7.52 (d, J=8.0 Hz, 1H), 7.47 (s, 1H), 7.39 (d, J=8.0 Hz, 1H), 3.55 (br.s., 1H), 3.44-3.38 (m, 1H), 3.17-2.95 (m, 4H), 2.58-2.44 (m, 2H), 2.14-2.11 (m, 2H), 1.89-1.75 (m, 4H), 1.45-1.31 (m, 4H), 1.20-1.14 (m, 2H), 0.78 (t, J=8.0 Hz, 2H).

### Example A25: Synthesis of Compound 84 (Isomer A)

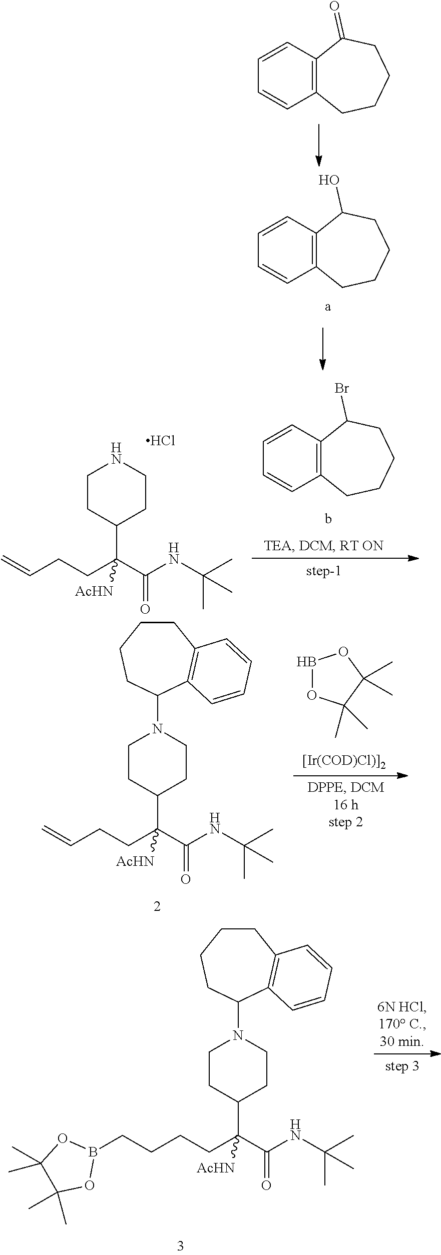

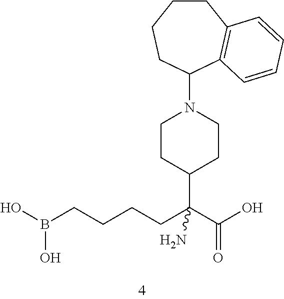 [SMILES: NC(CCCCB(O)O)(C(=O)O)C1CCN(C2CCCCc3ccccc32)CC1]

### Synthesis of intermediate a 6,7,8,9-tetrahydro-5H-benzo[7]annulen-5-ol

To the stirred solution of 6,7,8,9-tetrahydro-5H-benzo[7]annuen-5-one (1000 mg, 6.25 mmol) in methanol (5 mL) was added NaBH₄ (356 mg, 9.37 mmol, 1.5 eq.) portion wise at 0° C. under nitrogen atmosphere. After the completion of reaction (TLC monitoring) the reaction mixture was evaporated under reduced pressure to get crude residue which was then diluted with water (100 mL) and extracted with ethyl acetate (100 mL). The organic layer was separated dried over Na₂SO₄ filtered and evaporated under reduced pressure to yield 6,7,8,9-tetrahydro-5H-benzo[7]annuen-5-ol (800 mg). LCMS: 162.1 [M]⁺

### Synthesis of intermediate-b 5-bromo-6,7,8,9-tetrahydro-5H-benzo[7]annulene

To the stirred solution of 6,7,8,9-tetrahydro-5H-benzo[7]annuen-5-ol (800 mg, 4.24 mmol) in DCM (10 mL) was added PBr₃ (0.609 mL, 6.41 mmol) at 0° C. under nitrogen atmosphere. After the completion of reaction (TLC monitoring) the reaction mixture was evaporated under reduced pressure to get crude residue which was then diluted with water (100 mL) and extracted with ethyl acetate (100 mL). The organic layer was separated, dried over Na₂SO₄, filtered and evaporated under reduced pressure to get crude residue as 5-bromo-6,7,8,9-tetrahydro-5H-benzo[7]annulene. LCMS: 225.1 [M]⁺

### Synthesis of 2-acetamido-N-tert-butyl-2-(1-(6,7,8,9-tetrahydro-5H-benzo[7]annulen-5-yl)piperidin-4-yl)hex-5-enamide Isomer A

To the stirred solution of 5-bromo-6,7,8,9-tetrahydro-5H-benzo[7]annulene (300 mg, 0.86 mmol) in DCM (5 mL) was added triethylamine (0.4 mL, 2.58 mmol) and the reaction mixture was stirred for 10 minutes followed by addition of 2-acetamido-N-tert-butyl-2-(piperidin-4-yl)hex-5-enamide hydrochloride Isomer A (240 mg, 1.03 mmol) under nitrogen atmosphere and the reaction mixture was stirred for 16 h at RT. After the completion of reaction (LCMS monitoring) the reaction was diluted with water (100 mL) and extracted with EtOAc (200 mL). The organic layer was separated, dried over Na₂SO₄, filtered and evaporated under reduced pressure to give crude residue as 2-acetamido-N-tert-butyl-2-(1-(6,7,8,9-tetrahydro-5H-benzo[7]annulen-5-yl)piperidin-4-yl)hex-5-enamide Isomer A which was used as such in next step without any further purification. LCMS: 454.7 [M+H]⁺

### Synthesis of 2-acetamido-N-tert-butyl-2-(1-(6,7,8,9-tetrahydro-5H-benzo[7]annulen-5-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(6,7,8,9-tetrahydro-5H-benzo[7]annulen-5-yl)piperidin-4-yl)hex-5-enamide Isomer A (1.5 g as crude in DCM (10 mL) was added [Ir(COD)Cl]₂ (135 mg, 0.198 mmol) and DPPE (160 mg, 0.397 mmol) and the reaction mixture was stirred for 20 minutes at RT. After that the reaction mixture was then cooled to 0° C. and then 4,4,5,5-tetramethyl-1,3,2-dioxaborolane (0.57 mL, 3.97 mmol) was added and stirred for 16 h. After the completion of reaction (LCMS monitoring) the reaction was diluted with water (100 mL) and extracted with DCM (250 mL) the organic layer was separated, dried over Na₂SO₄, filtered and evaporated under reduced pressure to yield crude residue as 2-acetamido-N-tert-butyl-2-(1-(6,7,8,9-tetrahydro-5H-benzo[7]annulen-5-yl)piperidin-4-yl)-6-(4,4,5,5-tetramethyl-1,3,2-dioxaborolan-2-yl)hexanamide Isomer A. LCMS: 582.5 [M+H]⁺

### Synthesis of 2-amino-6-borono-2-(1-(6,7,8,9-tetrahydro-5H-benzo[7]annulen-5-yl)piperidin-4-yl)hexanoic acid Isomer A

A stirred solution of 2-acetamido-N-tert-butyl-2-(1-(6,7,8,9-tetrahydro-5H-benzo[7]annulen-5-yl)piperidin-4-yl)-6-(4,4,5,5-tetramethyl-1,3,2-dioxaborolan-2-yl) hexanamide Isomer A (2 g, 3.94 mmol) in 6 M HCl was subjected to microwave irradiation for 30 minutes at 170° C. After the completion of reaction (LCMS monitoring) the reaction mass was washed with DCM (50 mL) and the aqueous layer was lyophilized to get crude which was purified by reverse phase chromatography to yield 2-amino-6-borono-2-(1-(6,7,8,9-tetrahydro-5H-benzo[7]annulen-5-yl)piperidin-4-yl)hexanoic acid TFA salt Isomer A as a white solid (10 mg).

¹H NMR: (400 MHz, D₂O) δ 7.41 (dq, J=7.8, 4.8, 4.2 Hz, 1H), 7.37-7.28 (m, 3H), 3.88 (s, 1H), 3.34-3.26 (m, 1H), 3.22-3.09 (m, 1H), 3.08-2.95 (m, 1H), 2.84 (d, J=16 Hz, 2H), 2.29-2.19 (m, 1H), 2.18-2.06 (m, 2H), 2.05-1.97 (m, 1H), 1.94-1.75 (m, 4H), 1.74-1.51 (m, 4H), 1.49-1.31 (m, 4H), 1.28-1.18 (m, 2H), 0.80 (t, J=16 Hz, 2H).

### Example A26: Synthesis of Compound 85 (Isomer A)

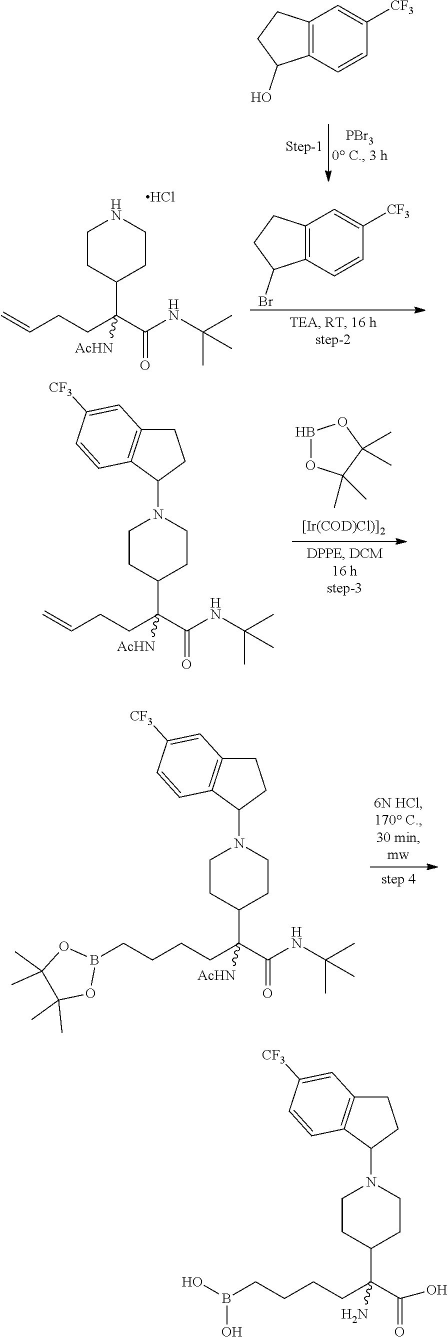

### Step-1: Synthesis of 1-bromo-5-(trifluoromethyl)indane

To a solution of 5-(trifluoromethyl)indan-1-ol (400 mg, 1.98 mmol, 1.0 eq.) in DCM (10 mL) was added phosphorous tribromide (0.24 mL, 2.57 mmol, 1.3 eq.) in DCM (3 mL) drop wise at 0° C. After completion of the addition the mixture was allowed to stir at RT for 3 h. Product formation was confirmed by NMR and after completion of reaction the reaction mixture was quenched by 5% solution of Na₂CO₃ in water and extracted with DCM (50 mL×3). Combined organic layer was dried over anhydrous Na₂SO₄ and concentrated under reduced pressure to yield 1-bromo-5-(trifluoromethyl)indane (395 mg, as crude) which was taken as such for next step without any further purification.

¹H NMR: (400 MHz, CHLOROFORM-d) δ ppm 7.55-7.47 (m, 3H), 5.54 (dd, J=6.14, 2.19 Hz, 1H), 3.32-3.16 (m, 1H), 2.96 (ddd, J=16.22, 7.67, 2.85 Hz, 1H) 2.71-2.42 (m, 2H)

### Step-2: Synthesis of 2-acetamido-N-tert-butyl-2-[1-[5-(trifluoromethyl)indan-1-yl]-4-piperidyl]hex-5-enamide Isomer A

To a stirred solution of 2-acetamido-N-tert-butyl-2-(4-piperidyl)hex-5-enamide hydrochloride Isomer A (150 mg, 0.43 mmol, 1.0 eq.) in DCM (5 mL) was added triethylamine (0.11 mg, 0.86 mmol, 2 eq.) at 0° C. The reaction mixture was allowed to stir at RT for 10 min and then 1-bromo-5-(trifluoromethyl)indane (158 mg, 0.56 mmol, 1.3 eq.) was added. The reaction mixture was stirred at RT for 16 h. Reaction progress was monitored by LCMS. Reaction was quenched by adding water and extracted with DCM (30 mL×3). Combined organic layer was dried over anhydrous Na₂SO₄ and concentrated under reduced pressure to obtain 2-acetamido-N-tert-butyl-2-[1-[5-(trifluoromethyl)indan-1-yl]-4-piperidyl]hex-5-enamide Isomer A (140 mg as a crude) which was directly used for next step without any further purification. LCMS: 494.3 [M+H]⁺

### Step-3: Synthesis of 2-acetamido-N-tert-butyl-6-(4,4,5,5-tetramethyl-1,3,2-dioxaborolan-2-yl)-2-[1-[5-(trifluoromethyl)indan-1-yl]-4-piperidyl]hexanamide Isomer A

To a stirred solution of 2-acetamido-N-tert-butyl-2-[1-[5-(trifluoromethyl)indan-1-yl]-4-piperidyl]hex-5-enamide Isomer A (140 mg, 0.31 mmol, 1.0 eq.) in DCM (5 mL) was added [Ir(COD)Cl]₂ (20 mg, 0.018 mmol, 0.06 eq.) and DPPE (5 mg, 0.009 mmol, 0.03 eq.). The reaction mixture was allowed to stir at RT for 15 min then cooled to 0° C. 4,4,5,5-tetramethyl-1,3,2-dioxaborolane (0.06 mL, 0.47 mmol, 1.5 eq.) was added drop wise at 0° C. After completion of addition the mixture was allowed to stir at RT for overnight. Reaction progress was monitored by TLC and NMR. The reaction mixture was diluted with ethyl acetate (30 mL) and washed with brine (40 mL). Organic layer was separated and dried over anhydrous Na₂SO₄ and concentrated under reduced pressure to get 2-acetamido-N-tert-butyl-6-(4,4,5,5-tetramethyl-1,3,2-dioxaborolan-2-yl)-2-[1-[5-(trifluoromethyl)indan-1-yl]-4-piperidyl]hexanamide Isomer A (140 mg, as crude) which was taken as such for next step without any further purification. LCMS: 622.5 [M+H]⁺

### Step-4: Synthesis of 2-amino-6-borono-2-[1-[5-(trifluoromethyl)indan-1-yl]-4-piperidyl]hexanoic acid Isomer A

A stirred solution of 2-acetamido-N-tert-butyl-6-(4,4,5,5-tetramethyl-1,3,2-dioxaborolan-2-yl)-2-[1-[5-(trifluoromethyl)indan-1-yl]-4-piperidyl]hexanamide Isomer A (140 mg as a crude) from previous step was dissolved in 6 N HCl and the mixture was heated at 170° C. in microwave reactor for 30 min. Product formation was confirmed by LCMS. The mixture was diluted with water (25 mL) and washed with DCM (15 mL×2). Aqueous phase was separated and freeze dried using a lyophilizer. The crude product was purified by reverse phase HPLC to obtain 2-amino-6-borono-2-[1-[5-(trifluoromethyl)indan-1-yl]-4-piperidyl]hexanoic acid TFA salt Isomer A (10.21 mg) as an off-white solid. LCMS: 425.4 [M+H−H₂O]⁺ & 443.4 [M+H]⁺

¹H NMR: (400 MHz, D₂O) δ 7.77 (s, 1H), 7.71 (q, J=8.2 Hz, 2H), 5.01 (d, J=8.1 Hz, 1H), 3.59 (t, J=11.1 Hz, 1H), 3.40 (t, J=10.4 Hz, 1H), 3.28-2.95 (m, 4H), 2.67-2.46 (m, 2H), 2.18-2.01 (m, 2H), 1.95-1.87 (m, 2H), 1.86-1.76 (m, 2H), 1.63-1.51 (m, 1H), 1.48-1.34 (m, 3H), 1.25-1.19 (m, 1H), 0.78 (t, J=7.7 Hz, 2H).

### Example A27: Synthesis of Compound 86 (Isomer A)

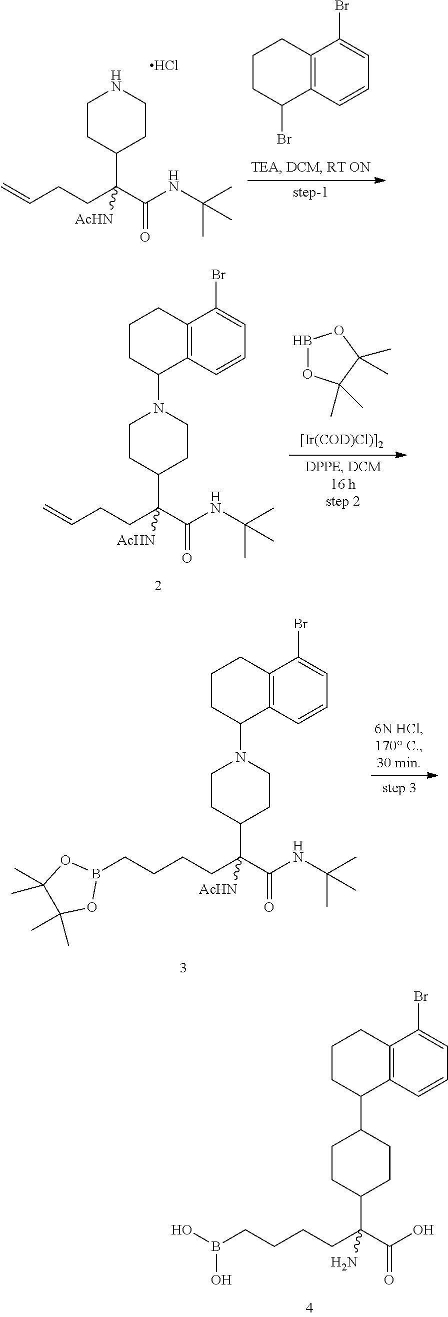

### Step-1: Synthesis of 2-acetamido-2-(1-(5-bromo-1,2,3,4-tetrahydronaphthalen-1-yl)piperidin-4-yl)-N-tert-butylhex-5-enamide Isomer A

To the stirred solution of 2-acetamido-N-tert-butyl-2-(piperidin-4-yl)hex-5-enamide hydrochloride Isomer A (100 mg, 0.28 mmol) in DCM (5 mL) was added triethylamine (0.2 mL, 0.56 mmol) and the reaction mixture was stirred for 10 minutes at RT. 1,5-dibromo-1,2,3,4-tetrahydronaphthalene (99 mg, 0.3 mmol) was added to the reaction mixture. After the completion of reaction (LCMS monitoring) the reaction mass was diluted with water (50 mL) and extracted with ethyl acetate (50 mL) the organic layer was separated, dried over anhydrous Na₂SO₄, filtered and evaporated under reduce pressure to get crude product which was used as such in next step without any further purification. LCMS: 518.2 [M]⁺

### Step-2: Synthesis of 2-acetamido-2-(1-(5-bromo-1,2,3,4-tetrahydronaphthalen-1-yl)piperidin-4-yl)-N-tert-butyl-6-(4,4,5,5-tetramethyl-1,3,2-dioxaborolan-2-yl)hexanamide Isomer A

To the stirred solution of 2-acetamido-2-(1-(5-bromo-1,2,3,4-tetrahydronaphthalen-1-yl)piperidin-4-yl)-N-tert-butylhex-5-enamide Isomer A (150 mg as crude) in DCM (5 mL) was added [Ir(COD)Cl]₂ (25 mg, 0.034 mmol) and DPPE (30 mg, 0.069 mmol) at RT under nitrogen atmosphere and the reaction mass was stirred for 10 minutes. After 10 minutes of stirring at RT the reaction mass was cooled to 0° C. then 4,4,5,5-tetramethyl-1,3,2-dioxaborolane (0.1 mL, 0.34 mmol) was added drop wise to the reaction mixture. After the completion of reaction mixture (LCMS monitoring) the reaction mixture was diluted with water (25 mL) and extracted with DCM (25 mL). The organic layer was separated, dried over Na₂SO₄, filtered and evaporated under reduced pressure to get crude residue. The crude was taken as such for next step without any further purification. LCMS: 646.4 [M]⁺

### Step-3: Synthesis of 2-amino-6-borono-2-(1-(5-bromo-1,2,3,4-tetrahydronaphthalen-1-yl)piperidin-4-yl)hexanoic acid Isomer A

A stirred solution of 2-acetamido-2-(1-(5-bromo-1,2,3,4-tetrahydronaphthalen-1-yl)piperidin-4-yl)-N-tert-butyl-6-(4,4,5,5-tetramethyl-1,3,2-dioxaborolan-2-yl)hexanamide Isomer A (200 mg as crude) in 6 M HCl (3 mL) was stirred in microwave for 30 minutes at 170° C. After the completion of reaction (LCMS monitoring) the reaction mixture was washed with DCM (50 mL) and the aqueous layer was lyophilized to get crude compound which was purified through reverse phase HPLC to yield the desired compound (TFA salt) as an off white solid (20.0 mg). LCMS: 449.1 [M+H−H₂O]⁺

¹H NMR: (400 MHz, D₂O) δ 7.75 (d, J=8.0 Hz, 1H), 7.45 (d, J=7.7 Hz, 1H), 7.24 (t, J=7.9 Hz, 1H), 3.60-3.57 (m, 2H), 3.23-3.04 (m, 2H), 2.88 (t, J=6.6 Hz, 2H), 2.32 (m, 1H), 2.28-2.06 (m, 3H), 2.0-1.81 (m, 5H), 1.68-1.37 (m, 5H), 1.27-1.15 (m, 2H), 0.80 (t, J=8 Hz, 2H).

### Example A28: Synthesis of Compound 87 (Isomer A)

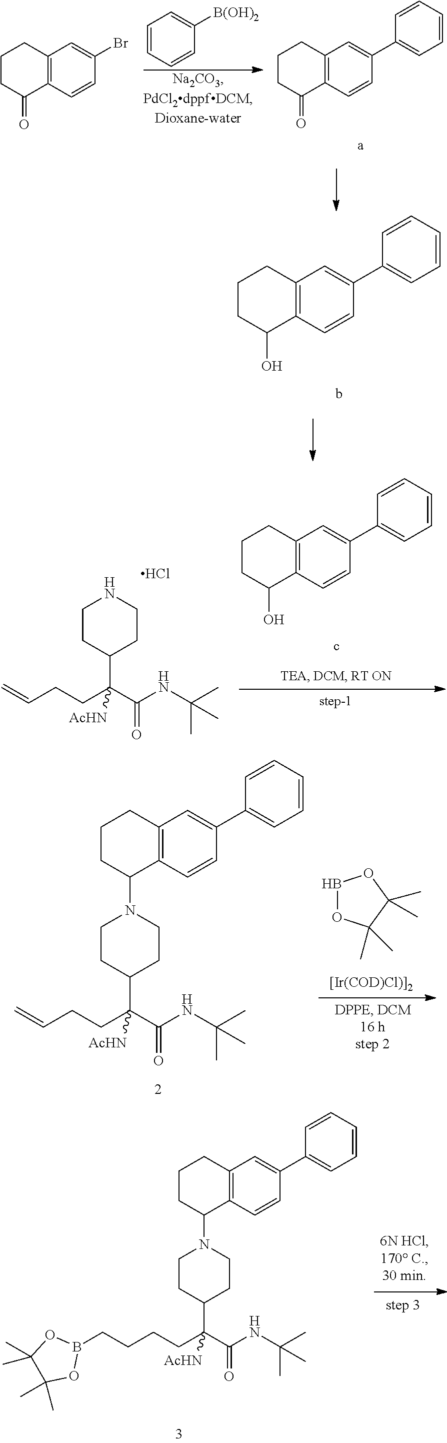

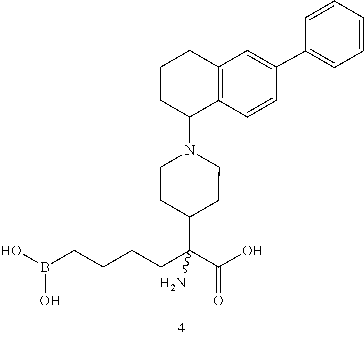 [SMILES: NC(CCCCB(O)O)(C(=O)O)C1CCN(C2CCCc3cc(-c4ccccc4)ccc32)CC1]

### Step-A: Synthesis of 6-phenyl-3,4-dihydronaphthalen-1(2H)-one

To the stirred solution of 6-bromo-1-tetralone (500 mg, 2.22 mmol) in 1,4 Dioxane:H₂O (15 mL, 3 mL) was added phenyl boronic acid (324 mg, 2.66 mmol) and Na₂CO₃ (470 mg, 4.44 mmol). The reaction mixture was then flushed with nitrogen for 15 minutes and then PdCl₂.dppf.DCM complex (174 mg, 0.22 mmol) was added. The reaction mixture was stirred for 16 h at 100° C. After the completion of reaction (TLC monitoring) the reaction mixture was filtered through celite pad and the mother liquor was washed with water (150 mL) and extracted with ethyl acetate (150 mL). The organic layer was separated, dried over Na₂SO₄ filtered and evaporated under reduced pressure to get crude residue which was purified using column chromatography. LCMS: 222.9 [M+H]⁺

¹H NMR: (400 MHz, D₂O) δ 8.10 (d, J=8 Hz, 1H), 7.67 (d, J=8 Hz, 2H), 7.53 (dd, J=8, 4 Hz, 1H), 7.49-7.46 (m, 3H), 7.4 (dt, J=4, 2.4 Hz, 1H), 3.04 (t, J=4 Hz, 2H), 2.69 (t, J=8 Hz, 2H), 2.18 (p, J=8 Hz, 2H).

### Synthesis of 6-phenyl-1,2,3,4-tetrahydronaphthalen-1-ol

To the stirred solution of 6-phenyl-3,4-dihydronaphthalen-1(2H)-one (450 mg, 2.02 mmol) in methanol (5 mL) was added NaBH₄ (87 mg, 2.43 mmol) portion wise at 0° C. and the reaction mixture was stirred for 30 minutes at RT under nitrogen atmosphere. After the completion of reaction (TLC monitoring) the reaction mixture was evaporated under reduced pressure to get crude product which was then diluted with water (50 mL) and extracted with ethyl acetate (50 mL). The organic layer was separated, dried over Na₂SO₄ filtered and evaporated under reduced pressure to get crude product which was as such in next step without further purification.

¹H NMR: (400 MHz, CDCl3) δ 7.59-7.55 (m, 2H), 7.51 (d, J=8 Hz, 1H), 7.46-7.4 (m, 3H), 7.34 (dt, J=12, 4 Hz, 2H), 4.84 (t, J=4 Hz, 1H), 2.95-2.75 (m, 2H), 2.04-1.9 (m, 2H), 1.87-1.76 (m, 2H), 1.75-1.71 (m, 1H).

### Synthesis of 1-bromo-6-phenyl-1,2,3,4-tetrahydronaphthalene

To the stirred solution of 6-phenyl-1,2,3,4-tetrahydronaphthalen-1-ol (300 mg, 1.33 mmol) in DCM (5 mL) was added PBr₃ (0.15 mL, 1.59 mmol) drop wise at 0° C. under nitrogen atmosphere and the reaction mixture was warmed to RT. After the completion of reaction (TLC monitoring) the reaction mixture was evaporated under reduced pressure to get crude residue. The crude reaction mixture was then diluted with water (50 mL) and extracted with ethyl acetate (50 mL). The organic layer was separated, dried over Na₂SO₄ filtered and evaporated under reduced pressure to get crude product which was used as such in next step without further purification.

¹H NMR: (400 MHz, D₂O) δ 7.59-7.55 (m, 2H), 7.46-7.32 (m, 5H), 7.31-7.28 (br.s., 1H), 5.66 (t, J=4 Hz, 1H), 3.06-2.84 (m, 2H), 2.49-2.13 (m 3H), 1.99-1.89 (m, 1H).

### Synthesis of 2-acetamido-N-tert-butyl-2-(1-(6-phenyl-1,2,3,4-tetrahydronaphthalen-1-yl)piperidin-4-yl)hex-5-enamide Isomer A

To the stirred solution of 2-acetamido-N-tert-butyl-2-(piperidin-4-yl)hex-5-enamide hydrochloride Isomer A (150 mg, 0.433 mmol) in DCM (3 mL) was added triethylamine (0.24 mL, 1.73 mmol) and the reaction mixture was stirred for 10 minutes at RT. Then 1-bromo-6-phenyl-1,2,3,4-tetrahydronaphthalene (400 mg as crude) was added to the reaction mixture and it was stirred for 16 h at RT under nitrogen atmosphere. After the completion of reaction (LCMS monitoring) the reaction mixture was diluted with water (50 mL) and extracted with DCM (50 mL). The organic layer was separated, dried with Na₂SO₄ filtered and evaporated under reduced pressure to get crude product which was used as such in next step without further purification. LCMS: 516.5 [M+H]⁺

### Synthesis of 2-acetamido-N-tert-butyl-2-(1-(6-phenyl-1,2,3,4-tetrahydronaphthalen-1-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(6-phenyl-1,2,3,4-tetrahydronaphthalen-1-yl)piperidin-4-yl)hex-5-enamide Isomer A (250 mg as crude) in DCM (3 mL) was added [Ir(COD)Cl]₂ (40 mg, 0.057 mmol) and DPPE (48 mg, 0.11 mmol) and the reaction mixture was stirred for 10 minutes and then brought to 0° C. 4,4,5,5-Tetramethyl-1,3,2-dioxaborolane (0.21 mL, 1.45 mmol) was added drop wise to the reaction mixture and the reaction mixture stirred for 16 h. After the completion of reaction mixture (LCMS monitoring) the reaction mixture was diluted with water (25 mL) and extracted with DCM (25 mL) the organic layer was separated, dried over Na₂SO₄, filtered and evaporated under reduced pressure to get crude product. The crude was taken as such for next step without any further purification. LCMS: 644.5 [M+H]⁺

### Synthesis of 2-amino-6-borono-2-(1-(6-phenyl-1,2,3,4-tetrahydronaphthalen-1-yl)piperidin-4-yl)hexanoic acid Isomer A

The solution of 2-acetamido-N-tert-butyl-2-(1-(6-phenyl-1,2,3,4-tetrahydronaphthalen-1-yl)piperidin-4-yl)-6-(4,4,5,5-tetramethyl-1,3,2-dioxaborolan-2-yl)hexanamide Isomer A (250 mg, 0.388 mol) in 5 M HCl (3 mL) was stirred at 100° C. under N₂ atmosphere. After the completion of reaction (LCMS monitoring) the reaction mixture was washed with DCM (50 mL) and the aqueous layer was lyophilized to get crude compound which was purified through reverse phase HPLC to yield desired compound (formate salt) as an off white solid (27 mg). LCMS: 447.5 [M+H−H₂O]⁺

¹H NMR: (400 MHz, D₂O) δ 7.72 (d, J=8 Hz, 2H), 7.65-7.60 (m, 2H), 7.59-7.52 (m, 3H), 7.51-7.45 (m, 1H), 3.68-3.59 (m, 1H), 3.58-3.44 (m, 1H), 3.21-3.08 (m, 2H), 2.95 (q, J=8 Hz, 2H), 2.28-2.21 (m, 2H), 2.19-2.10 (m, 2H), 2.09-1.8 (m, 6H), 1.72-1.52 (m, 2H), 1.5-1.3 (m, 3H), 1.26-1.15 (m, 1H), 0.8 (t, J=8 Hz, 2H).

### Example A29: Synthesis of Compound 88 (Isomer A1)

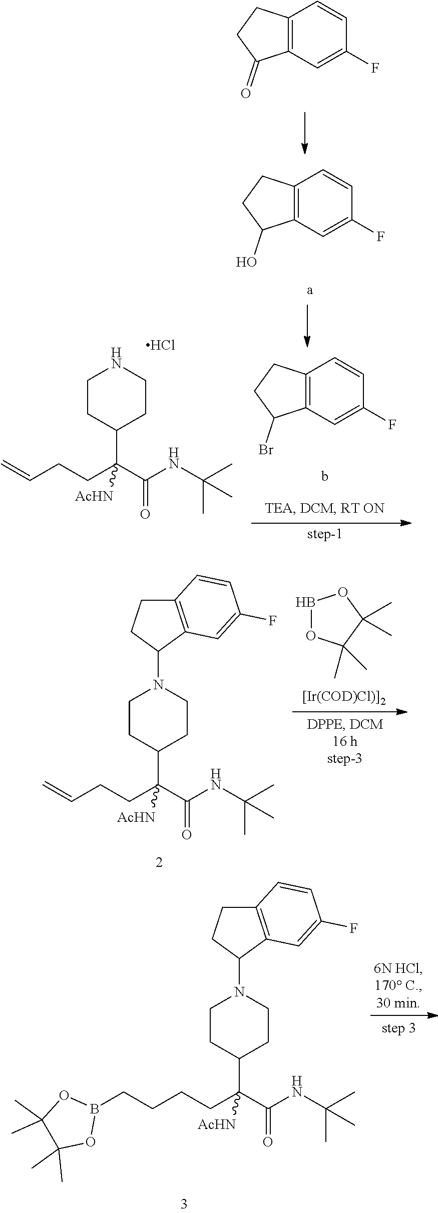

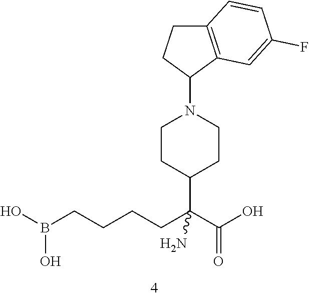

### Synthesis of 6-fluoro-2,3-dihydro-1H-inden-1-ol

To the stirred solution of 6-fluoro-2,3-dihydro-1H-inden-1-one (500 mg, 3.33 mmol) in methanol (5 mL) was added NaBH₄ (155 mg, 3.99 mmol) portion wise at 0° C. under nitrogen atmosphere and the reaction was stirred for 30 minutes at RT. After the completion of reaction (TLC monitoring) the reaction mass was evaporated under reduced pressure to get crude residue, the crude was then diluted with water (200 mL) and extracted with ethyl acetate (200 mL) the organic layer was separated, dried over Na₂SO₄, filtered and evaporated under reduced pressure to get crude residue. The crude product thus obtained was used as such in next reaction. LCMS: 135.0 [M+H−H₂O]⁺

### Synthesis of 1-bromo-6-fluoro-2,3-dihydro-1H-indene

To the stirred solution of 6-fluoro-2,3-dihydro-1H-inden-1-ol (500 mg, 3.28 mmol) in DCM (5 mL) was added PBr₃ (0.4 mL, 3.94 mmol) drop wise at 0° C. under nitrogen atmosphere and the reaction was stirred for 2 h at RT. After the completion of reaction (TLC monitoring) the reaction mixture was evaporated under reduced pressure to get crude product. The crude product was then diluted with water (150 mL) and extracted with ethyl acetate (150 mL). The organic layer was separated, dried over Na₂SO₄ filtered and evaporated reduced pressure to get crude product which was used as such in next reaction.

¹H NMR: (400 MHz, CHLOROFORM-d) δ ppm 7.19 (dd, J=8.11, 5.04 Hz, 1H) 7.10 (dd, J=8.77, 2.19 Hz, 1H) 6.95 (td, J=8.77, 2.63 Hz, 1H) 5.50 (dd, J=6.58, 2.19 Hz, 1H), 3.18-3.08 (m, 1H), 2.89-2.80 (m, 1H), 2.68-2.59 (m, 1H), 2.56-2.49 (m, 1H).

### Synthesis of 2-acetamido-N-tert-butyl-2-(1-(6-fluoro-2,3-dihydro-1H-inden-1-yl)piperidin-4-yl)hex-5-enamide Isomer A

To the stirred solution of 2-acetamido-N-tert-butyl-2-(piperidin-4-yl)-6-(4,4,5,5-tetramethyl-1,3,2-dioxaborolan-2-yl)hexanamide Isomer A (150 mg, 0.43 mmol) in DCM (5 mL) was added TEA (0.3 mL, 1.73 mmol) and stirred for 10 minutes at RT under nitrogen atmosphere then added 1-bromo-6-fluoro-2,3-dihydro-1H-indene (370 mg, 1.73 mmol) and reaction was stirred for 16 h at RT under nitrogen atmosphere. After the completion of reaction (LCMS monitoring) the reaction mixture was diluted with water (50 mL) and extracted with DCM (50 mL) the organic layer was separated, dried over Na₂SO₄, filtered and evaporated under reduced pressure to get crude residue. The crude product was used as such for next step. LCMS: 444.2 [M+H]⁺

### Synthesis of 2-acetamido-N-tert-butyl-2-(1-(6-fluoro-2,3-dihydro-1H-inden-1-yl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(6-fluoro-2,3-dihydro-1H-inden-1-yl)piperidin-4-yl)hex-5-enamide Isomer A (200 mg, 0.45 mmol) in DCM (5 mL) was added [Ir(COD)Cl]₂ (35 mg, 0.054 mmol) and DPPE (45 mg, 0.12 mmol) and the reaction mixture was stirred for 10 minutes and bought to 0° C., then added 4,4,5,5-tetramethyl-1,3,2-dioxaborolane (0.1 mL, 0.54 mmol) drop wise and the reaction mixture stirred for 16 h at RT. After the completion of reaction (LCMS monitoring) the reaction mixture was diluted with water (100 mL) and extracted with DCM (100 mL) the organic layer was separated, dried over Na₂SO₄, filtered and evaporated under reduced pressure to get crude product which was used as such for next step. LCMS: 572.5 [M+H]⁺

### Synthesis of 2-amino-6-borono-2-(1-(6-fluoro-2,3-dihydro-1H-inden-1-yl)piperidin-4-yl)hexanoic acid Isomer A

The solution of 2-acetamido-N-tert-butyl-2-(1-(6-fluoro-2,3-dihydro-1H-inden-1-yl)piperidin-4-yl)-6-(4,4,5,5-tetramethyl-1,3,2-dioxaborolan-2-yl)hexanamide Isomer A (100 mg, 0.175 mmol) in 5 M HCl (3 mL) was heated at 100° C. under nitrogen atmosphere. After the completion of reaction (LCMS monitoring) the reaction mixture was washed with DCM (50 mL) and the aqueous layer was lyophilized to get crude compound the crude was purified through reverse phase chromatography to get compound (formate salt) as off white solid (20 mg). LCMS: 375.2[M+H−H₂O]⁺

¹H NMR: (400 MHz, D₂O) δ 7.42 (dd, J=8.5, 5.2 Hz, 1H), 7.32 (dd, J=9.1, 2.5 Hz, 1H), 7.23 (td, J=8.9, 2.5 Hz, 1H), 4.94 (d, J=8.2 Hz, 1H), 3.63-3.52 (m, 1H), 3.41 (t, J=10.7 Hz, 1H), 3.19-2.93 (m, 4H), 2.65-2.42 (m, 2H), 2.19-2.01 (m, 2H), 1.98-1.74 (m, 4H), 1.67-1.30 (m, 4H), 1.23-1.14 (m, 1H), 0.79 (t, J=8.2 Hz, 2H).

### Example A30: Synthesis of Compound 89 (Isomer A)

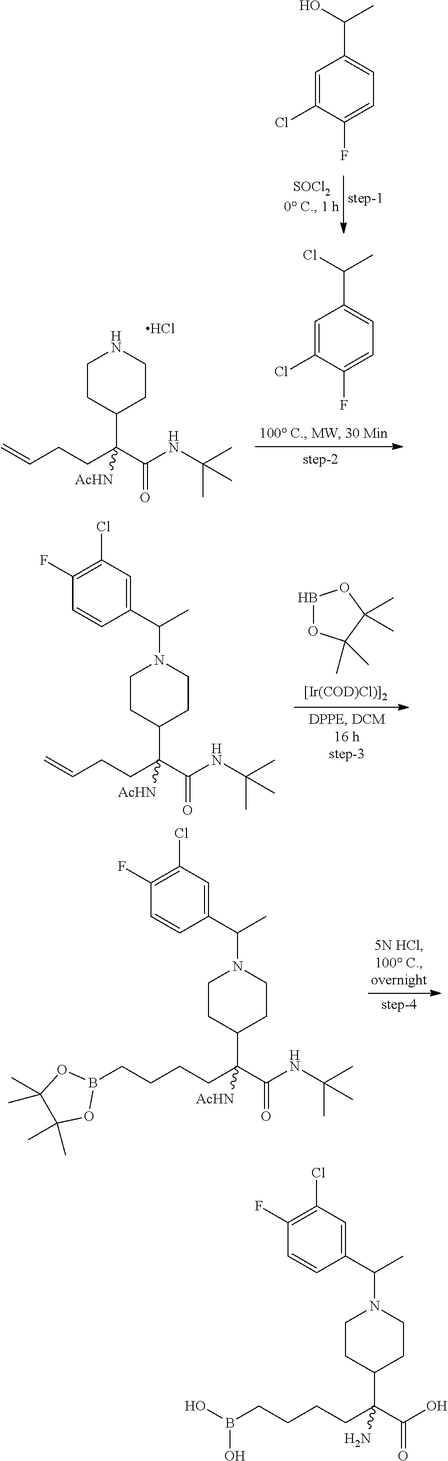

### Step-1: Synthesis of 2-chloro-4-(1-chloroethyl)-1-fluorobenzene

To a stirred solution of 1-(3-chloro-4-fluorophenyl)ethanol (800 mg, 5.2 mmol, 1.0 eq) in DCM (20 mL) was added SOCl₂ at 0° C. (1 mL, 13.2 mmol, 2.5 eq). The resultant solution was stirred at 0° C. for 1 h. Progress of the reaction was monitored through TLC. After completion of reaction, the reaction mixture was concentrated under reduced pressure, extracted with DCM (3×20 mL) and the organic layer was separated, filtered and evaporated in vacuo to obtain crude product which was used as such in the next step.

¹HNMR: (400 MHz, DMSO-d₆) ppm 7.73 (dd, J=7.02, 2.19 Hz, 1H) 7.50-7.58 (m, 1H) 7.37-7.47 (m, 1H) 5.31-5.43 (m, 1H) 1.78 (d, J=7.02 Hz, 3H).

### Step-2 Synthesis of 2-acetamido-N-tert-butyl-2-(1-(1-(3-chloro-4-fluorophenyl)ethyl) piperidin-4-yl)hex-5-enamide Isomer A

To a stirred solution of 2-acetamido-N-tert-butyl-2-(piperidin-4-yl)hex-5-enamide hydrochloride Isomer A (100 mg, 0.20 mmol, 1.0 eq) in DMF (2 mL), was added Cs₂CO₃ (282 mg, 0.86 mmol, 3.0 eq) and the reaction mixture was stirred for 10 minutes at RT. 2-Chloro-4-(1-chloroethyl)-1-fluoro-benzene (165 mg, 0.86 mmol, 3.0 eq) was then added to the reaction mixture. The resultant reaction mixture was subjected to microwave irradiation at 130° C. for 45 min. After the completion of reaction (LCMS monitoring) the reaction mass was diluted with water (50 mL) and extracted with ethyl acetate (3×20 mL) and the organic layer was separated, dried over anhydrous Na₂SO₄, filtered and concentrated in vacuo to obtain crude product which was used as such in the next step. LCMS: 466.2 [M]⁺

### Step-3: Synthesis of 2-acetamido-N-tert-butyl-2-(1-(1-(3-chloro-4-fluorophenyl)ethyl)piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(1-(3-chloro-4-fluorophenyl)ethyl)piperidin-4-yl)hex-5-enamide Isomer A (167 mg as crude) in DCM (20 mL) was added [Ir(COD)Cl]₂ (28 mg, 0.04 mmol) and DPPE (34 mg, 0.08 mmol) at RT under nitrogen atmosphere and the reaction mass was stirred for 10 minutes. After 10 minutes of stirring at RT the reaction mass was cooled to 0° C. and then 4,4,5,5-tetramethyl-1,3,2-dioxaborolane (55 mg, 0.4 mmol) was added drop wise to the reaction mixture. After overnight stirring at RT the reaction mixture was diluted with water (50 mL) and extracted with DCM (3×20 mL) the organic layer was separated, dried over Na₂SO₄, filtered and evaporated in vacuo to obtain crude product which was used as such in the next step. LCMS: 594.6 [M]⁺.

### Step-4: Synthesis of 2-amino-6-borono-2-(1-(1-(3-chloro-4-fluorophenyl)ethyl)piperidin-4-yl)hexanoic acid Isomer A

The stirred solution of 2-acetamido-N-tert-butyl-2-(1-(1-(3-chloro-4-fluorophenyl)ethyl) piperidin-4-yl)-6-(4,4,5,5-tetramethyl-1,3,2-dioxaborolan-2-yl)hexanamide Isomer A (160 mg, 0.2 mmol, 1.0 eq) in 5 M HCl (5 mL) was stirred at 100° C. for overnight. After the completion of reaction (LCMS monitoring) the reaction mixture was washed with DCM (3×20 mL) and the aqueous layer was lyophilized to get crude compound which was purified through reverse phase chromatography to get compound (formate salt) as an off white solid (15.8 mg). LCMS: 397.5 [M+H−H₂O]⁺

¹HNMR: (400 MHz, D₂O) 7.67 (d, J=6.7 Hz, 1H), 7.45 (d, J=7.0 Hz, 1H), 7.37 (t, J=8.9 Hz, 1H), 4.48 (d, J=9.8 Hz, 1H), 3.79 (d, J=11.4 Hz, 1H), 3.47 (d, J=10.9 Hz, 1H), 2.97 (q, J=11.3 Hz, 1H), 2.91-2.80 (m, 1H), 2.09 (m, 2H), 1.91-1.81 (m, 4H), 1.74 (d, J=6.6 Hz, 3H), 1.40 (m, 4H), 1.19 (dd, J=16.0, 8.7 Hz, 1H), 0.81 (q, J=11.4, 7.6 Hz, 2H).

### Example A31: Synthesis of Compound 90 (Isomer A)

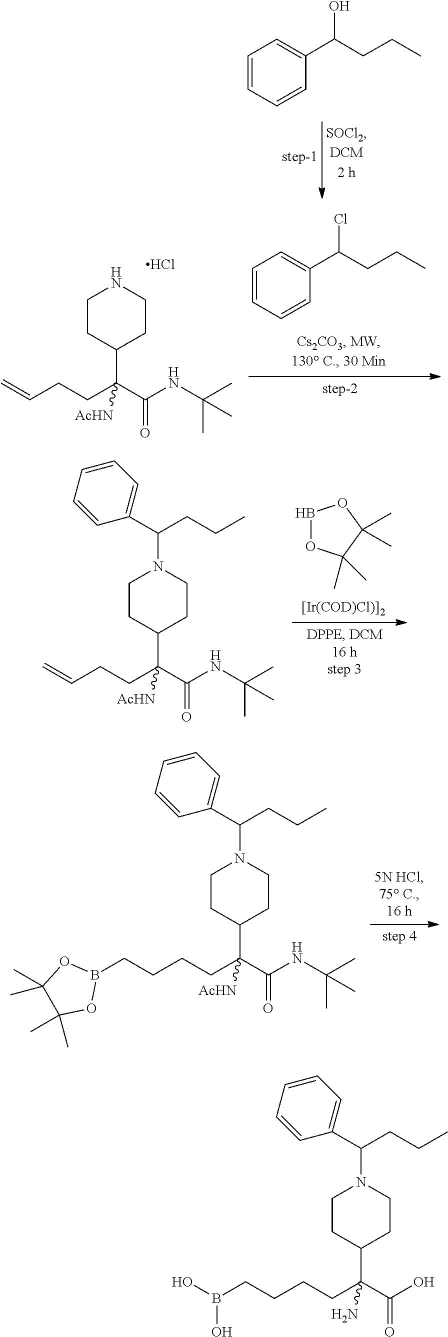

### Step-1: Synthesis of (1-chlorobutyl)benzene

To a solution of 1-phenylbutan-1-ol (500 mg, 3.33 mmol, 1.0 eq.) in DCM (10 mL) was added thionyl chloride (583 mg, 3.52 mmol, 1.5 eq.) in DCM (3 mL) drop wise at 0° C. After completion of addition the mixture was allowed to stir at RT for 3 h. Product formation was confirmed by NMR. After completion of reaction the reaction mixture was quenched by 5% aqueous solution of Na₂CO₃ and extracted with DCM (50 mL×3). Combined organic layer was dried over anhydrous Na₂SO₄ and concentrated under reduced pressure to obtain (1-chlorobutyl)benzene (600 mg as crude) which was directly taken for next step. LCMS: 168.2 [M]⁺

### Step-2: Synthesis of 2-acetamido-N-tert-butyl-2-[1-(1-phenylbutyl)-4-piperidyl]hex-5-enamide Isomer A

To a stirred solution of 2-acetamido-N-isopropyl-2-(4-piperidyl)hex-5-enamide hydrochloride Isomer A (125 mg, 0.362 mmol, 1.0 eq.) in DMF (5.0 mL) was added Cs₂CO₃ (224 mg, 0.724 mmol, 2.0 eq.) at RT, the reaction mixture was allowed to stir at RT for 10 min then (1-chlorobutyl)benzene (180 mg, 1.086 mmol, 3.0 eq.) was added to it and the reaction mixture was stirred under microwave irradiation at 130° C. for 30 min. The reaction progress was monitored by LCMS. Reaction was quenched by diluting it with water (10 mL) and extracted with EtOAc (50 mL×2). The organic layer was separated, dried over anhydrous Na₂SO₄ and concentrated under reduced pressure to obtain 2-acetamido-N-tert-butyl-2-[1-(1-phenylbutyl)-4-piperidyl]hex-5-enamide Isomer A (200 mg as crude) which was directly taken for next step. LCMS: 442.4 [M+H]⁺

### Step-3: Synthesis of 2-acetamido-N-tert-butyl-2-[1-(1-phenylbutyl)-4-piperidyl]-6-(4,4,5,5-tetramethyl-1,3,2-dioxaborolan-2-yl)hexanamide Isomer A

To a stirred solution of 2-acetamido-N-tert-butyl-2-[1-(1-phenylbutyl)-4-piperidyl]hex-5-enamide (200 mg as crude) in DCM (10 mL) was added [Ir(COD)Cl]₂ (80 mg, 0.054 mmol) and DPPE (43 mg, 0.108 mmol) and the reaction mixture was stirred for 15 minutes at RT. Then the reaction mixture was cooled to 0° C. and then 4,4,5,5-tetramethyl-1,3,2-dioxaborolane (69 mg, 0.542 mmol) was added and the reaction mixture was allowed to stir at RT. The reaction progress was monitored by LCMS. Reaction was quenched by diluting it with water (10 mL) and extracted with DCM (50 mL×2). The organic layer was separated, dried over anhydrous Na₂SO₄ and concentrated under reduced pressure to obtain 2-acetamido-N-tert-butyl-2-[1-(1-phenylbutyl)-4-piperidyl]-6-(4,4,5,5-tetramethyl-1,3,2-dioxaborolan-2-yl)hexanamide Isomer A (300 mg as crude) which was directly taken for next step. LCMS: 570.5 [M+H]⁺

### Step-4: Synthesis of 2-amino-6-borono-2-[1-(1-phenylbutyl)-4-piperidyl]hexanoic acid Isomer A

2-acetamido-N-tert-butyl-2-[1-(1-phenylbutyl)-4-piperidyl]-6-(4,4,5,5-tetramethyl-1,3,2-dioxaborolan-2-yl)hexanamide Isomer A (200 mg) was dissolved in 5N HCl and the reaction mixture was allowed to stir at 70° C. for 16 h. The progress of the reaction was monitored through LCMS. The mixture was diluted with water (100 mL) and washed with DCM (50 mL×2). The aqueous layer was separated and lyophilized to give crude as an off-white solid which was purified by reverse phase HPLC to obtain the final product 2-amino-6-borono-2-[1-(1-phenylbutyl)-4-piperidyl]hexanoic acid Isomer A as formate salt (off white solid, 5.0 mg). LCMS: 373.7 [M+H−H₂O]⁺

¹H NMR: (400 MHz, D₂O) δ 7.55-7.43 (m, 5H), 4.24 (d, J=10.8 Hz, 1H), 3.71 (d, J=8.5 Hz, 2H), 3.43 (d, J=4.3 Hz, 1H), 2.82 (d, J=12.3 Hz, 2H), 2.27-1.69 (m, 7H), 1.62-1.28 (m, 4H), 1.11 (m, 3H), 0.84 (t, J=8 Hz, 3H), 0.77 (t, J=4 Hz, 2H).

### Example A32: Synthesis of Compound 91 (Isomer A)

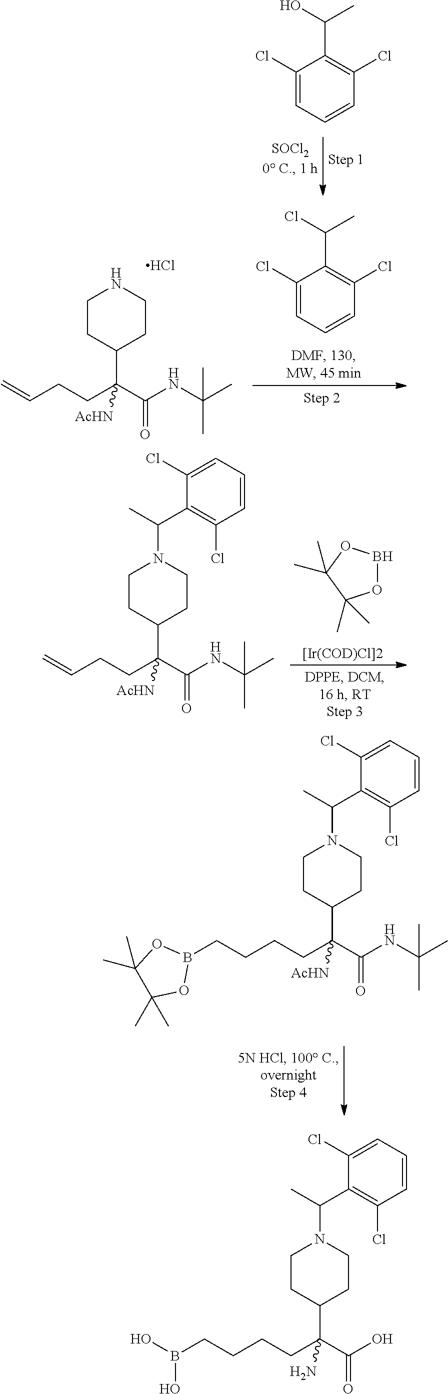

### Step-1: Synthesis of 2,6-dichloro-2-(1-chloroethyl)benzene

To a stirred solution of 1-(2,6 dichlorophenyl)ethanol (950 mg, 5.0 mmol 1.0 eq) in DCM (20 mL) SOCl₂ was added at 0° C. (1.2 mL 17.5 mmol 3.5 eq). The resultant reaction mixture was stirred at 0° C. for 1 h. Progress of the reaction was monitored through TLC. After completion of reaction, the reaction mixture was concentrated under reduced pressure, extracted with DCM (3×20 mL). The organic layer was separated, dried over anhydrous Na₂SO₄, filtered and evaporated under vacuo to give crude product which was used as such in next step without any purification.

¹HNMR: (400 MHz, DMSO-d₆) δ ppm 7.47-7.60 (m, 2H) 7.36-7.42 (m, 1H) 5.98 (q, J=7.02 Hz, 1H) 1.96 (d, J=7.02 Hz, 3H).

### Step-2: Synthesis of 2-acetamido-N-tert-butyl-2-(1-(1-(2,6-dichlorophenyl)ethyl)piperidin-4-yl)hex-5-enamide Isomer A

To a stirred solution of 2-acetamido-N-tert-butyl-2-(piperidin-4-yl)hex-5-enamide hydrochloride Isomer A (140 mg, 0.40 mmol, 1.0 eq) in DMF (2 mL), was added Cs₂CO₃ (520 mg, 1.6 mmol 4.0 eq) and the reaction mixture was stirred for 10 minutes at RT. Then 1,3-dichloro-2-(1-chloroethyl)benzene (509 mg, 2.4 mmol, 6.0 eq) was added to the reaction mixture. The resultant reaction mixture was subjected to microwave irradiation at 130° C. for 45 min. After the completion of reaction (LCMS monitoring) the reaction mass was diluted with water (50 mL) and extracted with ethyl acetate (3×20 mL) and the organic layer was separated, dried over anhydrous Na₂SO₄, filtered and concentrated in vacuo to obtain crude product which was used as such in the next step. LCMS: 482.3 [M]⁺

### Step-3: Synthesis of 2-acetamido-N-tert-butyl-2-(1-(1-(2,6-dichlorophenyl)ethyl)piperidin-4-yl)-6-(4,4,5,5-tetramethyl-1,3,2-dioxaborolan-2-yl)hexanamide Isomer A

To a stirred solution of 2-acetamido-N-tert-butyl-2-(1-(1-(2,6-dichlorophenyl)ethyl)piperidin-4-yl)hex-5-enamide Isomer A (200 mg as crude) in DCM (20 mL) was added [Ir(COD)Cl]₂ (33 mg, 0.049 mmol) and DPPE (39 mg, 0.098 mmol) at RT under nitrogen atmosphere and the reaction mixture was stirred for 10 minutes. After 10 minutes of stirring at RT the reaction mass was cooled to 0° C. and then 4,4,5,5-tetramethyl-1,3,2-dioxaborolane (63 mg, 0.49 mmol) was added drop wise to the reaction mixture. After overnight stirring at RT, the reaction mixture was diluted with water (50 mL) and extracted with DCM (3×20 mL) the organic layer was separated, dried over Na₂SO₄, filtered and evaporated in vacuo to obtain crude product which was used as such in the next step. LCMS: 610.5 [M]⁺

### Step-4: Synthesis of 2-amino-6-borono-2-(1-(1-(2,6-dichlorophenyl)ethyl)piperidin-4-yl)hexanoic acid Isomer A

The stirred solution of 2-acetamido-N-tert-butyl-2-(1-(1-(2,6-dichlorophenyl)ethyl)piperidin-4-yl)-6-(4,4,5,5-tetramethyl-1,3,2-dioxaborolan-2-yl)hexanamide Isomer A (300 mg as crude) in 5 M HCl (5 mL) was stirred at 100° C. for overnight. After the completion of reaction (LCMS monitoring) the reaction mixture was washed with DCM (3×20 mL) and the aqueous layer was lyophilized to get crude compound which was purified through reverse phase chromatography to get compound (formate salt) as off white solid (10.33 mg). LCMS: 413.5 [M−H₂O]⁺

¹HNMR: (400 MHz, D₂O) 7.56 (dd, J=20.7, 8.0 Hz, 2H), 7.44 (t, J=8.2 Hz, 1H), 4.19 (s, 1H), 3.40 (br.s., 1H), 3.28 (s, 1H), 2.99-2.88 (m, 1H), 2.27-2.14 (m, 1H), 2.03 (d, J=24.4 Hz, 2H), 1.87 (m, 6H), 1.48-1.33 (m, 4H), 1.19 (dd, J=17.5, 8.5 Hz, 1H), 0.81 (t, J=8 Hz, 3H).

### Example A33: Synthesis of Compound 92 (Isomer A)

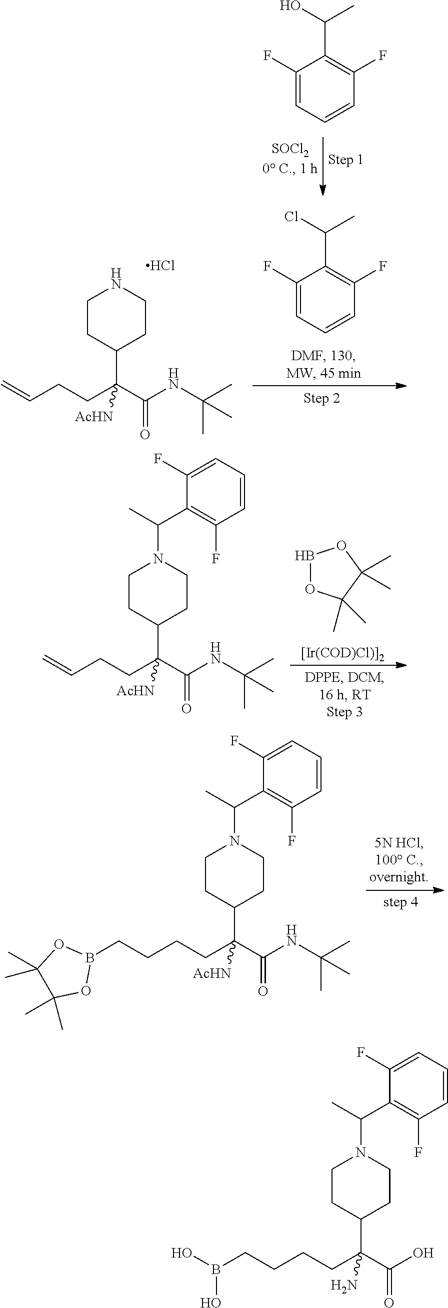

### Step-1: Synthesis of 2,6-difluoro-2-(1-chloroethyl)benzene

To a stirred solution of 1-(2,6 difluorophenyl) ethanol (1.2 g, 6.8 mmol, 1.0 eq) in DCM (20 mL) was added SOCl₂ at 0° C. (1.4 mL, 20.4 mmol, 3.5 eq) and the resultant reaction mixture was stirred at 0° C. for 1 h (monitored by TLC). After completion of reaction, it was concentrated under reduced pressure, extracted with DCM (3×20 mL). The organic layer was separated, dried over anhydrous Na₂SO₄, filtered and evaporated under vacuo to provide the crude product which was used as such in the next step.

¹HNMR: (400 MHz, DMSO-d₆) δ ppm 7.47 (tt, J=8.55, 6.58 Hz, 1H) 7.05-7.22 (m, 2H) 5.57 (q, J=7.02 Hz, 1H) 1.89 (d, J=7.02 Hz, 3H).

### Step-2: Synthesis of 2-acetamido-N-tert-butyl-2-(1-(1-(2,6-difluorophenyl)ethyl)piperidin-4-yl)hex-5-enamide Isomer A

To the stirred solution of 2-acetamido-N-tert-butyl-2-(4-piperidyl)hex-5-enamide hydrochloride Isomer A (150 mg, 0.43 mmol, 1.0 eq) in DMF (2 mL), was added Cs₂CO₃ (549 mg, 1.72 mmol, 4.0 eq) and the reaction mixture was stirred for 10 minutes at RT. 2,6-difluoro-2-(1-chloroethyl)benzene (459 mg, 2.6 mmol, 6.0 eq) was added to the reaction mixture and it was subjected to MW irradiation at 130° C. for 45 min. After the completion of reaction (LCMS monitoring) the reaction mass was diluted with water (50 mL) and extracted with ethyl acetate (3×20 mL). The organic layer was separated, dried over anhydrous Na₂SO₄, filtered and evaporated under reduce pressure to get crude residue which was used as such in the next step without any purification. LCMS: 450.4 [M+H]⁺

### Step-3 Synthesis of 2-acetamido-N-tert-butyl-2-(1-(1-(2,6-difluorophenyl)ethyl) piperidin-4-yl)-6-(4,4,5,5-tetramethyl-1,3,2-dioxaborolan-2-yl)hexanamide Isomer A

To the stirred solution of 2-acetamido-N-tert-butyl-2-(1-(1-(2,6-difluorophenyl)ethyl)piperidin-4-yl)hex-5-enamide (150 mg, 0.3 mmol, 1.0 eq) in DCM (20 mL) was added [Ir(COD)Cl]₂ (24 mg, 0.036 mmol, 0.12 eq) and DPPE (28 mg, 0.072 mmol, 0.24 eq) at RT under nitrogen atmosphere and the reaction mass was stirred for 10 minutes. After 10 minutes of stirring at RT the reaction mass was cooled to 0° C. and then 4,4,5,5-tetramethyl-1,3,2-dioxaborolane (42 mg, 0.33 mmol, 1.2 eq) was added drop wise to the reaction mixture. After the completion of reaction mixture (LCMS monitoring) the reaction mixture was diluted with water (50 mL) and extracted with DCM (3×20 mL). The organic layer was separated, dried over Na₂SO₄, filtered and evaporated under reduced pressure to get crude residue. The crude reaction mixture was taken as such for the next step. LCMS: 578.5 [M+H]⁺

### Step-4: Synthesis of 2-amino-6-borono-2-(1-(1-(2,6-difluorophenyl)ethyl)piperidin-4-yl)hexanoic acid Isomer A

The solution of 2-acetamido-N-tert-butyl-2-(1-(1-(2,6-difluorophenyl)ethyl)piperidin-4-yl)-6-(4,4,5,5-tetramethyl-1,3,2-dioxaborolan-2-yl)hexanamide Isomer A (200 mg, 0.43 mmol, 1.0 eq) in 5 M HCl (6 mL) was stirred at 100° C. for overnight. After the completion of reaction (LCMS monitoring) the reaction mixture was washed with DCM (3×20 mL) and the aqueous layer was lyophilized to get crude compound which was purified through reverse phase HPLC to get the desired compound (formate salt) as off white solid (43 mg). LCMS: 363.2 [(M+H−(2×H₂O)]⁺

¹H NMR: (400 MHz, D₂O) δ 7.55 (p, J=8 Hz, 1H), 7.13 (t, J=8 Hz, 2H), 3.94 (t, J=8 Hz, 1H), 3.80 (t, J=8 Hz, 1H), 3.17-3.03 (m, 1H), 3.03-2.87 (m, 1H), 2.25-1.85 (m, 5H), 1.83 (t, J=4 Hz, 3H), 1.75-1.48 (m, 1H), 1.46-1.30 (m, 5H), 1.26-1.11 (m, 1H), 0.94-0.76 (m, 2H).

### Biological Examples

### Example B1: Arginase Enzymatic Assay In Vitro

Arginase catalyzes the hydrolysis of L-arginine into L-ornithine and urea. Urea amount produced by this reaction can be detected using a colorimetric assay and used as an indirect measurement of arginase activity. An adapted protocol from (Baggio, R., et al., J Pharmacol Exp Ther, 1999. 290(3): p. 1409-16) was used for the testing the ability of exemplary compounds of the invention to inhibit arginase enzymes from a variety of sources including recombinant human ARG1 and 2, lysates of human red blood cells (RBC) and intact murine macrophages, as described below.

Compounds of the invention were prepared from powder as 10 mM stock solutions in DMSO and stored in presence of N2 neutral atmosphere at −80° C. For IC₅₀ determination and just before the assay, exemplary compounds were successively diluted to get the following concentrations: 210, 70, 21, 7, 2.1, 0.7, 0.21, 0.07, 0.021, 0.007, 0.0021 and 0.0007 μM (i.e., final concentrations in the wells of 30, 10, 3, 1, 0.3, 0.1, 0.03, 0.01, 0.003, 0.001, 0.0003, 0.0001 μM).

Assays were run in clear flat bottom 96-well plates (Nunclon, Cat. No. #167008) adding in each well the following solutions in this order: (1) 10 μl of Tris-HCl solution supplemented with 0.1% bovine serum albumin (BSA) containing 1.563 μg/ml recombinant human ARG1 (R&D System, Cat. No. #5868-AR-010), (2) 30 μl of Tris-HCl buffer containing 10 mM MnCl2 and (3) 10 μL of a pre-dilution of each test article or DMSO (vehicle). Compounds were allowed to interact with the enzyme-cofactor complex pre-incubating the plate at 37° C. for 30 min. To start the reaction, 20 μl of 50 mM L-arginine (Sigma Aldrich Cat. #A5006-100G) were added to each well in exception of the blank reaction (see below). All reactions were allowed to happen for 1 h at 37° C. To detect the urea production, reactions were stopped by adding 200 μl of a mixture of reagent A (4 mM o-phthalaldehyde in 50 mM Boric Acid, 1 M Sulfuric Acid, 0.03% Brij-35 (w/v)) and reagent B (4 mM N-(1-naphthyl)ethylenediamine in 50 mM Boric Acid, 1 M Sulfuric Acid, 0.03% Brij-35 (w/v)) in equal proportion. Color product was developed for 1 h at room temperature (RT) protected from light and then absorbance at 520 nm was measured using a Multifunction Microplate Reader (Synergy 4, Bioteck). Blank reactions were carried out by adding the L-arginine substrate only after the addition of mixture A-B. All reactions were carried out in duplicates.

The inhibition of urea production was calculated subtracting the absorbance from the blank reaction (zero enzymatic urea production) to the absorbance from all reactions and it was expressed as the percentage of the absorbance from the reaction containing the test article with respect to the absorbance from vehicle reaction (maximum of urea production).

For testing inhibitory activity over recombinant human arginase II, 10 μl of Tris-HCl solution supplemented with 0.1% BSA containing 1.5 μg/ml recombinant human ARG2 (BPS Bioscience, Cat. No. #71659) was used instead of ARG1.

For testing inhibitory activity over RBC lysates from healthy donors, 10 μl of Tris-HCl solution supplemented with 0.1% BSA containing 160 μg RBC lysates per well was used instead.

Results of exemplary compounds for inhibition of recombinant human ARG1/2 enzymes and native ARG1 enzyme in lysates of RBC are shown in Table 1.

TABLE 1
Potency of exemplary compounds for inhibition
of human ARG 1/2 activities

| | IC₅₀ Inh | IC₅₀ Inh | IC₅₀ Inh |
|---|---|---|---|
| Compound | rhARG1 | rhARG2 | RBC |
| Number | [μM] | [μM] | [μM] |
| 1 | +++ | ++ | +++ |
| 2 | +++ | +++ | +++ |
| (Isomer A, | | | |
| Example A2) | | | |
| 3 | + | + | + |
| (Isomer B, | | | |
| Example A3) | | | |
| 4 | +++ | ++ | +++ |
| 5 | +++ | +++ | +++ |
| (Isomer A, | | | |
| Example A5) | | | |
| 6 | + | + | + |
| (Isomer B, | | | |
| Example A6) | | | |
| 7 | +++ | ++ | +++ |
| (Isomer C, | | | |
| Example A7) | | | |
| 8 | +++ | +++ | +++ |
| (Isomer D, | | | |
| Example A7) | | | |
| 9 | +++ | ++ | +++ |
| 10 | +++ | +++ | +++ |
| (Isomer A, | | | |
| Example A9) | | | |
| 11 | + | + | + |
| (Isomer B, | | | |
| Example A9) | | | |
| 14 | +++ | +++ | +++ |
| (Isomer A, | | | |
| Example A10) | | | |
| 16 | +++ | +++ | +++ |
| 17 | +++ | +++ | +++ |
| (Isomer A, | | | |
| Example A12) | | | |
| 19 | +++ | +++ | − |
| (Isomer A, | | | |
| Example A13) | | | |
| 23 | + | + | − |
| 24 | ++ | ++ | − |
| (Isomer A, | | | |
| Example A15) | | | |
| 25 | + | + | − |
| (Isomer B, | | | |
| Example A16) | | | |
| 32 | +++ | +++ | +++ |
| (Isomer A, | | | |
| Example A17) | | | |
| 35 | +++ | +++ | − |
| (Isomer A, | | | |
| Example A18) | | | |
| 48 | ++ | ++ | − |
| 77 | +++ | ++ | +++ |
| 79 | +++ | +++ | +++ |
| (Isomer A, | | | |
| Example A21) | | | |
| 81 | ++ | ++ | +++ |
| 82 | +++ | ++ | − |
| 83 | +++ | +++ | +++ |
| (Isomer A, | | | |
| Example A24) | | | |
| 84 | +++ | ++ | +++ |
| (Isomer A, | | | |
| Example A25) | | | |
| 85 | +++ | +++ | +++ |
| (Isomer A, | | | |
| Example A26) | | | |
| 86 | +++ | ++ | − |
| (Isomer A, | | | |
| Example A27) | | | |
| 87 | +++ | +++ | − |
| (Isomer A, | | | |
| Example A28) | | | |
| 88 | +++ | +++ | − |
| (Isomer A, | | | |
| Example A29) | | | |
| 89 | +++ | ++ | − |
| (Isomer A, | | | |
| Example A30) | | | |
| 90 | ++ | ++ | − |
| (Isomer A, | | | |
| Example A31) | | | |
| 91 | +++ | ++ | − |
| (Isomer A, | | | |
| Example A32) | | | |
| 92 | ++ | ++ | − |
| (Isomer A, | | | |
| Example A33) | | | |

+++ refers to IC₅₀ < 0.5 μM; ++ refers to 0.5 μM < IC₅₀ < 5 μM; + refers to IC₅₀ > 5 μM; − represents compounds not tested; rhARG1: recombinant human arginase 1; rhARG2: recombinant human arginase 2; Inh: inhibition; IC₅₀: half maximal inhibitory concentration; RBC: Red blood cell.

### Example B2: Cell-Based Arginase Assay

Tumor-associated macrophages (TAMs) are the dominant leukocyte population infiltrating the tumor and play critical role in modulating the tumor microenvironment (Yang, L., et al., J Hematol Oncol, 2017. 10(1): p. 58). Monocytes/macrophages can be polarized to M1 or M2 phenotype. Classically activated macrophages (M1-polarized macrophages) are activated by cytokines such as interferon-γ, produces pro-inflammatory and immunostimulatory cytokines (e.g., interleukin[IL]-12 and IL-23), and are involved in helper T cell (Th) 1 responses to infection. TAMs are thought to more closely resemble M2-polarized macrophages (Grivennikov, S. I., et al., Cell, 2010. 140(6): p. 883-99), also known as alternatively activated macrophages, which are activated by Th2 cytokines (e.g., interleukin (IL)-4, IL-10, and IL-13).

The M1/M2 macrophages use different metabolic pathways for arginine degradation. The preference of macrophages to metabolize arginine via nitric oxide synthase (NOS) to NO and citrulline or via Arginase to ornithine and urea defines them as M1 (NOS) or M2 (arginase) respectively (Mills, C. D., Crit Rev Immunol, 2012. 32(6): p. 463-88).

Murine bone-marrow derived macrophages (BMM) represent a convenient in vitro model that enables differentiation of these cells towards M1 or M2 macrophages. Briefly, male C57BL/6 mice 7-8 weeks old were euthanized by carbon dioxide asphyxiation. Femur and tibiae bones were removed under aseptic conditions and collected in a plate containing sterile ice-cold PBS 1×. After sterilization of bones in 70% ethanol during 1 min, bones were placed into a clean Petri dish containing fresh sterile ice-cold PBS 1×. The ends of each bone were cut and the marrow cavity was flushed using forceps and syringes filled with pre-warmed complete α-MEM growing medium (α-MEM without red phenol (Life Technologies, Cat. No. #41061-029) supplemented with 10% FBS, penicillin and streptomycin). Bone marrow (BM) cells were harvested in a 15 ml clean tube and centrifuged at 400 g for 8 min at RT. Supernatant was discarded and BM pellet was re-suspended in 5 ml Ammonium-Chloride-Potassium (ACK) buffer for 5 minutes at RT to lysis RBC. BM cell suspension was centrifuged at 400 g for 8 min at RT. Supernatant was discarded and BM cell pellet was re-suspended in complete α-MEM growing medium and passed through a 70 μm cell restrainer to eliminate solid contaminates and cellular aggregates. BM cells were seeded in 8 ml of complete α-MEM growing medium supplemented with 100 ng/ml recombinant mouse M-CSF (R&D Systems, Cat. No. #416-ML-050) in 100-mm dishes. BM cells were cultured overnight to get rid of contaminating stromal cells in a humidified incubator with 5% CO₂ at 37° C. At the following day, non-adherent cells were collected in a clean 15-ml tube and centrifuged at 400 g for 8 min at RT. Cell pellet was re-suspended in complete α-MEM growing medium and counted using a hemocytometer Neubauer's chamber. Hematopoietic precursor cells were differentiated into BMM in presence of complete α-MEM growing medium supplemented 50 ng/ml M-CSF for 3 days. Finally to polarize BMM towards M2 phenotype, cells were incubated in presence 20 ng/ml of recombinant mouse IL-4 (R&D Systems, Cat. No. #404-ML-010) added to fresh complete α-MEM growing medium supplemented 50 ng/ml M-CSF for 24 h.

At the day of the experiment, BMM-M2 were treated for 24 h with vehicle (DMSO) or compounds of the invention at 1 and 10 μM prepared in fresh complete α-MEM growing medium supplemented 50 ng/ml M-CSF, 20 ng/ml IL-4 and 10 mM L-arginine (Cat. No. #A5006, Sigma Aldrich). At the next day, 70 μl aliquots of each treated culture media (supernatants) were transferred into a clear 96-well plate. Measurements of urea produced by BMM-M2 were performed using the adapted protocol detailed in Example B1. Wells without BMM-M2 containing only treatment media (vehicle) were used to determine the zero cell-derived urea production (blank). All treatments were carried out in triplicates.

The inhibition of urea production was calculated subtracting the absorbance from the blank to the absorbance from all treatments and it was expressed as the percentage of the absorbance from the reaction containing the test article treatment with respect to the absorbance from vehicle treatment (maximum of urea production).

Results of exemplary compounds for inhibiting urea production by cultured BMM-M2 cells are shown in Table 2.

TABLE 2
Potency of exemplary compounds for inhibiting
arginase activity in intact BMM-M2 cells.

| Compound | Inh. urea production |
|---|---|
| Number | @10 μM (%) |
| 1 | +++ |
| 2 | +++ |
| (Isomer A, | |
| Example A2) | |
| 3 | + |
| (Isomer B, | |
| Example A3) | |
| 4 | +++ |
| 5 | +++ |
| (Isomer A, | |
| Example A5) | |
| 6 | + |
| (Isomer B, | |
| Example A6) | |
| 7 | +++ |
| (Isomer C, | |
| Example A7) | |
| 8 | +++ |
| (Isomer D, | |
| Example A7) | |
| 9 | ++ |
| 10 | +++ |
| (Isomer A, | |
| Example A9) | |
| 11 | + |
| (Isomer B, | |
| Example A9) | |
| 14 | +++ |
| (Isomer A, | |
| Example A10) | |
| 16 | − |
| 17 | +++ |
| (Isomer A, | |
| Example A12) | |
| 19 | ++ |
| (Isomer A, | |
| Example A13) | |
| 23 | − |
| 24 | ++ |
| (Isomer A, | |
| Example A15) | |
| 25 | − |
| (Isomer B, | |
| Example A16) | |
| 32 | − |
| (Isomer A, | |
| Example A17) | |
| 35 | + |
| (Isomer A, | |
| Example A18) | |
| 48 | + |
| 77 | ++ |
| 79 | +++ |
| (Isomer A, | |
| Example A21) | |
| 81 | − |
| 82 | +++ |
| 83 | +++ |
| (Isomer A, | |
| Example A24) | |
| 84 | +++ |
| (Isomer A, | |
| Example A25) | |
| 85 | +++ |
| (Isomer A, | |
| Example A26) | |
| 86 | ++ |
| (Isomer A, | |
| Example A27) | |
| 87 | +++ |
| (Isomer A, | |
| Example A28) | |
| 88 | +++ |
| (Isomer A, | |
| Example A29) | |
| 89 | ++ |
| (Isomer A, | |
| Example 30) | |
| 90 | ++ |
| (Isomer A, | |
| Example A31) | |
| 91 | +++ |
| (Isomer A, | |
| Example A32) | |
| 92 | + |
| (Isomer A, | |
| Example A33) | |

+++ refers to % inhibition > 25 at 10 μM test compound; ++ refers to 25% < % inhibition < 15% at 10 μM test compound; + refers to % inhibition < 15 at 10 μM.

### Example B3—Oral Bioavailability and PK Parameters

The pharmacokinetics of the exemplary compounds of the invention were studied after administration of an intravenous (IV) 2 mg/kg or oral (PO) 10 mg/kg single dose at a single time point (30 min) in mice. Compounds were formulated at 0.4 and 1 mg/ml in a vehicle containing Poly-Ethylene Glycol 400 (PEG400; Cat. No. #91893, Sigma Aldrich) as dosing solutions for intravenous and oral administration, respectively. Male C57BL6 mice, approximately 8-10 weeks old (22-25 grams), were obtained from the vivarium Fundación Ciencia & Vida Chile (Santiago, Chile) and maintained in a temperature-controlled room with 12/12 hr light/dark schedule with food and water ad libitum. Animals were acclimated for a minimum period of 4 days upon arrival at the testing facility.

At the day of study, mice were weighed, identified by marking the tail with numbers using a non-toxic permanent marker and designated into the experimental groups (n=3 per group). Each mouse of IV dosing groups received the systemic administration of 2 mg/kg dosing solution via caudal vein. Each mouse of PO dosing groups received the intragastric administration of 10 mg/kg via feeding tubes 20 G (Cat. No.: FTP-2038; Instech Salomon Inc.).

Blood samples were harvested by terminal cardiac puncture at 30 min after dosing. Non-dosed mice were used to collect samples of zero time points. Whole blood was collected into microtainer tubes with (K2) EDTA (Cat. No.: 365974, Becton Dickinson & Co.). Blood samples were centrifuged immediately at 9,000 g at 4° C. for 5 min and then plasmas were separated. Plasma samples were placed into individually labeled cryovials and stored in a −80° C. freezer until LC/MS/MS bioanalysis.

The bioanalysis of plasma samples to determine concentrations of exemplary compounds of the invention was conducted with a QTRAP 4500 triple quadrupole mass spectrometer (Applied Biosystems SCIEX) in the positive ion mode and interfaced with an ekspert ultraLC 100-XL UPLC System (eksigent). Calibration standards (0.01 to 100 μM) and QCs (0.2, 2.0 and 20 μM) were prepared from naïve mouse plasma in parallel with mouse plasma study samples (60 μl) by precipitation with three volumes of ice cold internal standard solution (acetonitrile containing 20 μM of theophylline). The precipitated samples were centrifuged at 6,100 g for 30 min at 4° C. Following centrifugation, an aliquot of each supernatant was transferred into a clean sample vial and diluted with two volumes of aqueous mobile phase (0.2% formic acid in water). Samples were injected onto a reverse phase analytical column (YMC Triart C18; 2.0×50 mm; 1.9 μm; YMC CO) and eluted with a gradient of 0.1 or 0.2% formic acid in Acetonitrile. Test compound and internal standard were monitored by a multiple reaction monitoring (MRM) experiment using an Analyst software (v 1.6.2, Applied Biosystems SCIEX). Quantitation was conducted using a MultiQuant software (v 2.1, Applied Biosystems SCIEX) and the resulting calibration curve was fitted with a linear or quadratic regression and 1/× weighting. The lower limit of quantitation were between 0.01-0.03 μM.

IV and PO PK parameters were calculated from the concentration-time data using Phoenix WinNonlin software (v 6.4, Certara, Princeton, N.J.) by noncompartmental analysis in the sparse sampling mode.

Results for concentration of exemplary compounds in mouse plasma are shown in Table 3.

TABLE 3
Plasma concentrations of exemplary compounds
after IV or PO dosing in mice

| | 2 mg/Kg IV | 11 mg/Kg PO |
|---|---|---|
| Compound | @ t = 30 min | @ t = 30 min |
| Number | [nM] | [nM] |
| 2 | +++ | + |
| (Isomer A, | | |
| Example A2) | | |
| 5 | ++ | + |
| (Isomer A, | | |
| Example A5) | | |
| 10 | + | + |
| (Isomer A, | | |
| Example A9) | | |
| 83 | +++ | + |
| (Isomer A, | | |
| Example A24) | | |

++ refers to plasma concentration > 1000 nM for test compound; ++ refers to 500 < plasma concentration < 1000 nM for test compound; + refers to plasma concentration < 500 nM.

### Example B4—T Cell/Myeloid Cell Co-Culture Assays

To determine if compounds can restore lymphocyte proliferation in the context of immunosuppressive arginase-expressing myeloid cells, T cell proliferation is assayed in co-culture with human myeloid cells in the presence or absence of exemplary compounds of the invention.

Granulocytes are purified from peripheral blood of healthy donor using a pan-granulocyte negative selection kit (Stemcell Technologies) and incubated in SILAC-RPMI medium containing 10% charcoal-stripped FBS, antibiotics/anti-mycotic, 0.27 mM L-lysine, 20 μM MnCl2, 100 μM L-arginine, pH 7.4, plus different concentrations of exemplary compounds for 48 h at 37° C., during which time they spontaneously activate as determined by increased surface expression of CD66b and scatter properties. T cells are isolated from the same healthy donor using a pan-T cell isolation kit (Stemcell Technologies), loaded with CFSE and plated with immobilized anti-CD3 and soluble anti-CD28 in the presence of the aged granulocytes. Cells are co-cultured at several ratios of granulocytes to T cells or at a fixed ratio of 4 T cells to 1 granulocyte. Co-cultures are incubated for 3-4 days, at which time culture media (supernatants) is analyzed for urea production and T cell proliferation by flow cytometry.

Granulocytic Myeloid Derived Suppressor Cells (G-MDSC) or granulocytes from cancer patients are isolated from whole blood (Conversant Biologics). G-MDSCs are purified from the PBMC layer of a Ficoll gradient by positive selection for CD66b+ cells. Granulocytes are purified from the RBC layer of a Ficoll gradient using Hetasep (Stemcell Technologies). Granulocytes and G-MDSCs are characterized by flow cytometry for CD66b expression. Freshly isolated G-MDSC or granulocytes will be incubated in coculture medium containing 100 μM L-arginine for 48 h, at which time the cells are removed and G-MDSC- or granulocyte-conditioned media is used for incubating healthy donor CFSE-loaded T cells on immobilized anti-CD3/soluble anti-CD28 for 3-4 days at which time supernatant is analyzed for urea production and T cell proliferation by flow cytometry.

### Example B5—Tumor Pharmacodynamic Effects

To determine the pharmacokinetic and pharmacodynamic of exemplary compounds in the tumor one or more of the following in vivo tumor models are performed:

LLC model: Female C57B1/6 mice are implanted subcutaneously with 1×106 Lewis Lung Carcinoma cells suspended in PBS.

4T1 model: Female balb/c mice are implanted in the mammary fat pad with 1×105 4T1 mammary carcinoma cells suspended in PBS.

CT26 model: Female balb/c mice are implanted subcutaneously with 1×106 CT26 colon carcinoma cells suspended in PBS.

B16-F10 model: Female C57B1/6 mice are implanted subcutaneously with 2×106 B16-F10 murine melanoma cells suspended in PBS.

On day 8-14 post-engraftment, tumor-bearing mice are randomized into groups of n=5-7 mice and are treated with a single oral or intraperitoneal dose of vehicle or exemplary compounds at 50 mg/kg. Two hours post-dose, mice are sacrificed and plasma samples and tumors are collected and flash frozen in liquid nitrogen. Concentrations of exemplary compounds and L-arginine in plasma and tumor homogenate are determined by LC/MS/MS.

### Example B6—Tumor and Liver Multi-Dose Pharmacodynamic Effects

To determine anti-tumor efficacy, exemplary compounds will be tested one or more of the following syngeneic murine models of cancer:

4T1 model: Female Balb/c mice are implanted in the mammary fat pad with 1×105 4T1 mammary carcinoma cells suspended in PBS. The day following implant groups of n=10 mice are dosed PO or IP twice daily (bis in die, BID) for 21 days with vehicle or exemplary compound at 50 or 100 mg/kg.

LLC model: Female C₅₇BL/6 mice (n=20) are implanted subcutaneously with 1×106 Lewis Lung Carcinoma cells suspended in PBS. The day following implantation, mice are randomized into two experimental groups (n=10) to receive the following treatments dosed PO or IP BID: Vehicle or exemplary compound at 100 mg/kg.

Tumors are measured three times per week with digital calipers and tumor volumes calculated with the following formula:

Tumor volume (mm³)=(a×b2/2),

where ‘b’ is the smallest diameter and ‘a’ is the largest perpendicular diameter.

On Day 21, n=5 mice per groups are sacrificed at the trough time-point (−16 hrs following the previous dose), and the remaining n=5 mice per group receive a final dose and are sacrificed two hours from the last dose. At sacrifice, plasma, tumors and liver are harvested and flash frozen in liquid nitrogen. Concentrations of exemplary compounds and L-arginine in plasma, tumor and liver homogenates are determined by LC/MS/MS.

### Example B7—Combination Therapy Efficacy Study

Effective immune response against tumors may be blocked by more than one suppressive mechanism, including expression of immune checkpoint proteins and depletion of essential nutrients from the TME (Cotechini, T., et al., Cancer J, 2015. 21(4): p. 343-50; Spranger, S., et al., J Immunother Cancer, 2013. 1: p. 16; Munn, D. H., et al., Curr Opin Immunol, 2016. 39: p. 1-6) the combination of exemplary compounds with other immunomodulating or other anti-cancer agents are tested one or more of the following combinational approaches:

Checkpoint blockade therapies: anti-PD1, anti-PD-L1 or anti-CTLA-4 are test in LLC, CT26 or MC38 tumor-bearing mice.

Adoptive cell transfer therapy: PMEL specific T cells are test in B16-F10 tumor-bearing mice. Adoptive NK cell therapy is tested in a CT26 tumor-bearing mice.

Chemotherapy: Gemcitabine combinatory is tested in CT26, LLC, or 4T1 tumor-bearing mice.

What is claimed is:

1. A compound of Formula (I): 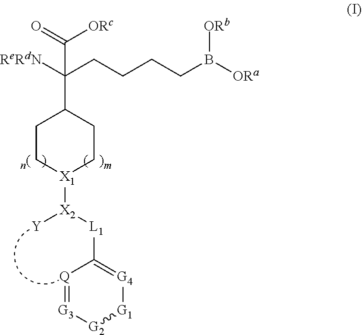
or a pharmaceutically acceptable salt, stereoisomer or tautomer thereof,
wherein:
X₁ and X₂ are independently N or CH;
Y is CR¹R², CR¹R²R³, —O—, —OR⁴, —S—, —SR⁵, —NR⁶—, or —NR⁶R⁷;
Q is C or CR⁸; [symbol]
is absent or C₁-C₄ alkylene, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo;
provided that: (i) [symbol] is C₁-C₄ alkylene, taken together with Y and Q to form a ring, when Y is CR¹R² and Q is C; and (ii) [symbol] is absent when Y is CR¹R²R³ and Q is CR⁸;
L₁ is a bond or C₁-C₄ alkylene, wherein the C₁-C₄ alkylene is unsubstituted or substituted with 1, 2, 3, or 4 substituents selected from the group consisting of halogen, C₁-C₆ alkyl, C₃-C₈ cycloalkyl, —CN, —OH, and oxo;
G₁ is CR^{1a}, C(O), N or NH;
G₂ is CR^{2a}, C(O), N or NH; [symbol]
is a single bond or a double bond, provided that: (i) [symbol] is a single bond when G₁ is C(O) and G₂ is NH or when G₂ is C(O) and G₁ is NH; and (ii) [symbol] is a double bond when G₁ is CR^{1a} and G₂ is CR^{2a} or N, and when G₁ is N and G₂ is CR^{2a};
G₃ is CR^{3a} or N;
G₄ is CR^{4a} or N;
m and n are independently 0, 1 or 2, provided that at least one of m and n is 1 or 2;
R^{a} and R^{b} are each independently hydrogen, C₁-C₆ alkyl, or C₃-C₈ cycloalkyl, wherein the C₁-C₆ alkyl and C₃-C₈ cycloalkyl are each independently unsubstituted or substituted with 1, 2, 3, or 4 substituents selected from the group consisting of halogen, C₁-C₆ alkyl, C₃-C₈ cycloalkyl, —CN, —OH, and oxo;
or R^{a} and R^{b} are taken together with the atoms to which they are attached to form a 5- to 10-membered heterocyclyl, wherein the 5- to 10-membered heterocyclyl is unsubstituted or substituted with 1, 2, 3, or 4 substituents selected from the group consisting of halogen, C₁-C₆ alkyl, C₃-C₈ cycloalkyl, —CN, —OH, and oxo;
R^{c}, R^{d} and R^{e} are each independently hydrogen, C₁-C₆ alkyl, C₃-C₈ cycloalkyl, or C₆-C₁₄ aryl, wherein the C₁-C₆ alkyl, C₃-C₈ cycloalkyl and C₆-C₁₄ aryl are each independently unsubstituted or substituted with 1, 2, 3, or 4 substituents selected from the group consisting of halogen, C₁-C₆ alkyl, C₃-C₈ cycloalkyl, —CN, —OH, and oxo;
R¹, R², R³, R⁴, R⁵, R⁶, R⁷, and R⁸ are each independently hydrogen, halo, C₁-C₆ alkyl, or C₃-C₈ cycloalkyl, wherein the C₁-C₆ alkyl and C₃-C₈ cycloalkyl are each independently unsubstituted or substituted with 1, 2, 3, or 4 substituents selected from the group consisting of halogen, C₁-C₆ alkyl, C₃-C₈ cycloalkyl, —CN, —OH, and oxo;
R^{1a}, R^{2a}, R^{3a} and R^{4a} are each independently hydrogen, halo, —CN, —OR^{f}, NR^{g}R^{h}, C₁-C₆ alkyl, C₂-C₆ alkenyl, C₂-C₆ alkynyl, C₃-C₈ cycloalkyl, or C₆-C₁₄ aryl, wherein the C₁-C₆ alkyl, C₂-C₆ alkenyl, C₂-C₆ alkynyl, C₃-C₈ cycloalkyl and C₆-C₁₄ aryl are each independently unsubstituted or substituted by 1, 2, 3, or 4 groups independently selected from the group consisting of halogen, C₁-C₆ alkyl, C₃-C₈ cycloalkyl, —CN, —OH, and oxo; and
R^{f}, R^{g}, and R^{h} are each independently hydrogen, C₁-C₆ alkyl, C₃-C₈ cycloalkyl, or C₆-C₁₄ aryl, wherein the C₁-C₆ alkyl, C₃-C₈ cycloalkyl and C₆-C₁₄ aryl are each independently unsubstituted or substituted with 1, 2, 3, or 4 substituents selected from the group consisting of halogen, C₁-C₆ alkyl, C₃-C₈ cycloalkyl, —CN, —OH, and oxo.

2. The compound of claim 1, or a pharmaceutically acceptable salt, stereoisomer, or tautomer thereof, wherein X₁ is N and X₂ is CH.

3. The compound of claim 1, or a pharmaceutically acceptable salt, stereoisomer, or tautomer thereof, wherein X₁ is CH and X₂ is N.

4. The compound of claim 1, or a pharmaceutically acceptable salt, stereoisomer, or tautomer thereof, wherein X₁ and X₂ both are N.

5. The compound of any one of claims 1-4, or a pharmaceutically acceptable salt, stereoisomer, or tautomer thereof, wherein [symbol] is absent, Y is CR¹R²R³ and Q is CR⁸.

6. The compound of claim 5, or a pharmaceutically acceptable salt, stereoisomer, or tautomer thereof, wherein Y is CH₃ and Q is CH.

7. The compound of any one of claims 1-4, or a pharmaceutically acceptable salt, stereoisomer, or tautomer thereof, wherein [symbol] is C₁-C₄ alkylene, Y is CR¹R², Q is C, and [symbol] is taken together with Y and Q to form a ring.

8. The compound of claim 7, or a pharmaceutically acceptable salt, stereoisomer, or tautomer thereof, wherein [symbol] is C₁-C₂ alkylene, Y is CR¹R², Q is C, and [symbol] is taken together with Y and Q to form a 5- or 6-membered ring.

9. The compound of any one of claims 1-8, or a pharmaceutically acceptable salt, stereoisomer, or tautomer thereof, wherein L₁ is C₁-C₂ alkylene, wherein the C₁-C₂ alkylene is unsubstituted or substituted with 1, 2, 3, or 4 substituents selected from the group consisting of halogen, C₁-C₆ alkyl, C₃-C₈ cycloalkyl, —CN, —OH, and oxo.

10. The compound of any one of claims 1-9, or a pharmaceutically acceptable salt, stereoisomer, or tautomer thereof, wherein [symbol] is a double bond, G₁ is CR^{1a} and G₂ is CR^{2a}.

11. The compound of claim 10, or a pharmaceutically acceptable salt, stereoisomer, or tautomer thereof, wherein R^{1a} and R^{2a} are each independently hydrogen, halo, —CN, —OR^{f}, NR^{g}R^{h}, C₁-C₆ alkyl, or C₆-C₁₄ aryl.

12. The compound of claim 10, or a pharmaceutically acceptable salt, stereoisomer, or tautomer thereof, wherein R^{1a} and R^{2a} are each independently hydrogen, halo, C₁-C₆ alkyl, or C₆-C₁₄ aryl.

13. The compound of claim 10, or a pharmaceutically acceptable salt, stereoisomer, or tautomer thereof, wherein R^{1a} and R^{2a} are each independently hydrogen, or halo.

14. The compound of claim 10, or a pharmaceutically acceptable salt, stereoisomer, or tautomer thereof, wherein R^{1a} and R^{2a} are each independently —H, —F, —Cl, —Br, —CH₃, —CH₂F, —CF₃, —CH₂OH, —CN, or —NH₂.

15. The compound of any one of claims 1-9, or a pharmaceutically acceptable salt, stereoisomer, or tautomer thereof, wherein [symbol] is a double bond, G₁ is CR^{1a} and G₂ is N.

16. The compound of claim 15, or a pharmaceutically acceptable salt, stereoisomer, or tautomer thereof, wherein R^{1a} is hydrogen, halo, —CN, —OR^{f}, NR^{g}R^{h}, C₁-C₆ alkyl, or C₆-C₁₄ aryl.

17. The compound of claim 15, or a pharmaceutically acceptable salt, stereoisomer, or tautomer thereof, wherein R^{1a} is hydrogen, halo, C₁-C₆ alkyl, or C₆-C₁₄ aryl.

18. The compound of claim 15, or a pharmaceutically acceptable salt, stereoisomer, or tautomer thereof, wherein R^{1a} is hydrogen, or halo.

19. The compound of claim 15, or a pharmaceutically acceptable salt, stereoisomer, or tautomer thereof, wherein R^{1a} is —H, —F, —Cl, —Br, —CH₃, —CH₂F, —CF₃, —CH₂OH, —CN, or —NH₂.

20. The compound of any one of claims 1-9, or a pharmaceutically acceptable salt, stereoisomer, or tautomer thereof, wherein [symbol] is a double bond, G₁ is N and G₂ is CR^{2a}.

21. The compound of claim 20, or a pharmaceutically acceptable salt, stereoisomer, or tautomer thereof, wherein R^{2a} is hydrogen, halo, —CN, —OR^{f}, NR^{g}R^{h}, C₁-C₆ alkyl, or C₆-C₁₄ aryl.

22. The compound of claim 20, or a pharmaceutically acceptable salt, stereoisomer, or tautomer thereof, wherein R^{2a} is hydrogen, halo, C₁-C₆ alkyl, or C₆-C₁₄ aryl.

23. The compound of claim 20, or a pharmaceutically acceptable salt, stereoisomer, or tautomer thereof, wherein R^{2a} is hydrogen, or halo.

24. The compound of claim 20, or a pharmaceutically acceptable salt, stereoisomer, or tautomer thereof, wherein R^{2a} is —H, —F, —Cl, —Br, —CH₃, —CH₂F, —CF₃, —CH₂OH, —CN, or —NH₂.

25. The compound of any one of claims 1-9, or a pharmaceutically acceptable salt, stereoisomer, or tautomer thereof, wherein [symbol] is a single bond, G₁ is C(O) and G₂ is NH.

26. The compound of any one of claims 1-9, or a pharmaceutically acceptable salt, stereoisomer, or tautomer thereof, wherein [symbol] is a single bond when G₂ is C(O) and G₁ is NH.

27. The compound of any one of claims 1-26, or a pharmaceutically acceptable salt, stereoisomer or tautomer thereof, wherein G₃ is CR^{3a}.

28. The compound of claim 27, or a pharmaceutically acceptable salt, stereoisomer, or tautomer thereof, wherein R^{3a} is hydrogen, halo, —CN, —OR^{f}, NR^{g}R^{h}, C₁-C₆ alkyl, or C₆-C₁₄ aryl.

29. The compound of claim 27, or a pharmaceutically acceptable salt, stereoisomer, or tautomer thereof, wherein R^{3a} is hydrogen, halo, C₁-C₆ alkyl, or C₆-C₁₄ aryl.

30. The compound of claim 27, or a pharmaceutically acceptable salt, stereoisomer, or tautomer thereof, wherein R^{3a} is hydrogen, or halo.

31. The compound of claim 27, or a pharmaceutically acceptable salt, stereoisomer, or tautomer thereof, is —H, —F, —Cl, —Br, —CH₃, —CHF, —CF₃, —CH₂OH, —CN, —NH₂.

32. The compound of any one of claims 1-26, or a pharmaceutically acceptable salt, stereoisomer, or tautomer thereof, wherein G₃ is N.

33. The compound of any one of claims 1-32, or a pharmaceutically acceptable salt, stereoisomer, or tautomer thereof, wherein G₄ is CR^{4a}.

34. The compound of claim 33, or a pharmaceutically acceptable salt, stereoisomer, or tautomer thereof, wherein R^{4a} is hydrogen, halo, —CN, —OR^{f}, NR^{g}R^{h}, C₁-C₆ alkyl, or C₆-C₁₄ aryl.

35. The compound of claim 33, or a pharmaceutically acceptable salt, stereoisomer, or tautomer thereof, wherein R^{4a} is hydrogen, halo, C₁-C₆ alkyl, or C₆-C₁₄ aryl.

36. The compound of claim 33, or a pharmaceutically acceptable salt, stereoisomer, or tautomer thereof, wherein R^{4a} is hydrogen, or halo.

37. The compound of claim 33, or a pharmaceutically acceptable salt, stereoisomer, or tautomer thereof, wherein R^{4a} is —H, —F, —Cl, —Br, —CH₃, —CHF, —CF₃, —CH₂OH, —CN, —NH₂.

38. The compound of any one of claims 1-32, or a pharmaceutically acceptable salt, stereoisomer, or tautomer thereof, wherein G₄ is N.

39. The compound of any one of claims 1-9, or a pharmaceutically acceptable salt, stereoisomer, or tautomer thereof, wherein [symbol] is a double bond, G₁, G₂, G₃, and G₄ all are CH.

40. The compound of any one of claims 1-39, or a pharmaceutically acceptable salt, stereoisomer, or tautomer thereof, wherein 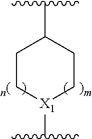
is selected from the group consisting of

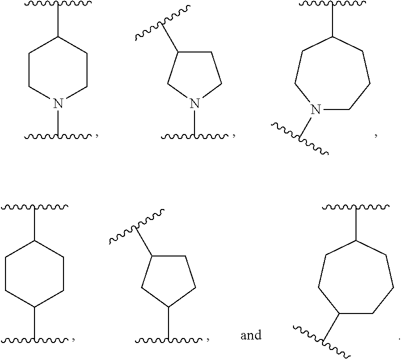 [SMILES: *C1CCN(*)CC1 ; *C1CCN(*)C1 ; *C1CCCN(*)CC1 ; *C1CCC(*)CC1 ; *C1CCC(*)C1 ; *C1CCCC(*)CC1]

41. The compound of any one of claims 1-40, or a pharmaceutically acceptable salt, stereoisomer, or tautomer thereof, wherein m is 1.

42. The compound of any one of claims 1-41, or a pharmaceutically acceptable salt, stereoisomer, or tautomer thereof, wherein n is 1.

43. The compound of any one of claims 1-42, or a pharmaceutically acceptable salt, or tautomer thereof, wherein the carbon bearing the —NR^{d}R^{e} and —COOR^{c} moieties is in the “R” configuration.

44. The compound of any one of claims 1-42, or a pharmaceutically acceptable salt, or tautomer thereof, wherein the carbon bearing the —NR^{d}R^{e} and —COOR^{c} moieties is in the “S” configuration.

45. The compound of any one of claims 1-44, or a pharmaceutically acceptable salt, stereoisomer or tautomer thereof, wherein the compound is of Formula (IIa) or Formula (IIb): 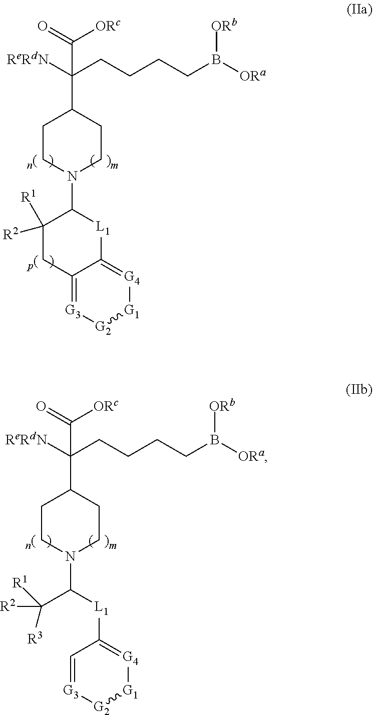
wherein p is 1 or 2.

46. The compound of any one of claims 1-44, or a pharmaceutically acceptable salt, stereoisomer or tautomer thereof, wherein the compound is of Formula (IIIa) or Formula (IIIb): 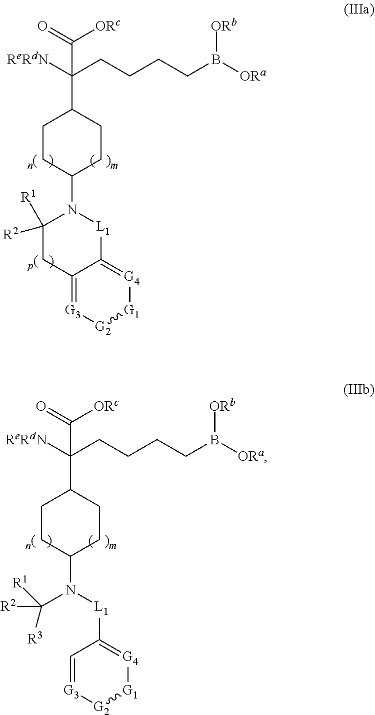
wherein p is 1 or 2.

47. The compound of any one of claims 1-44, or a pharmaceutically acceptable salt, stereoisomer or tautomer thereof, wherein the compound is of Formula (IVa) or Formula (IVb): 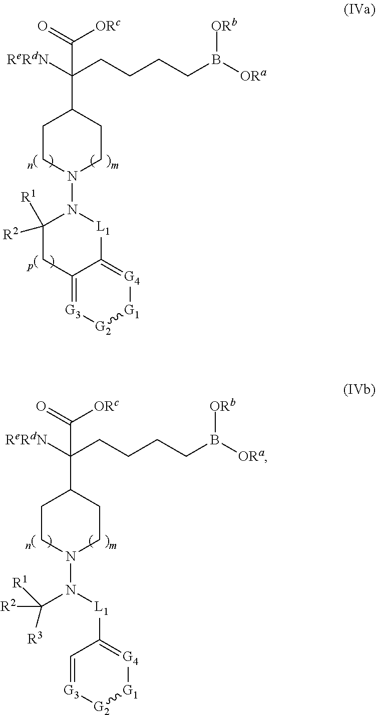
wherein p is 1 or 2.

48. The compound of claim 1, or a pharmaceutically acceptable salt, stereoisomer or tautomer thereof, wherein the compound is selected from the group consisting of

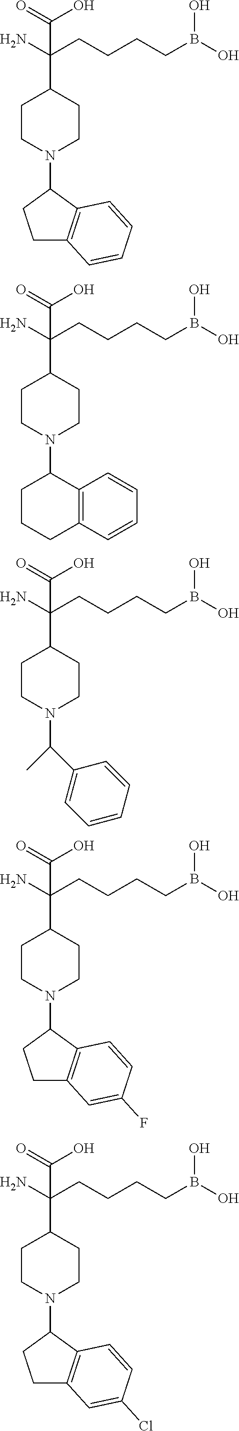

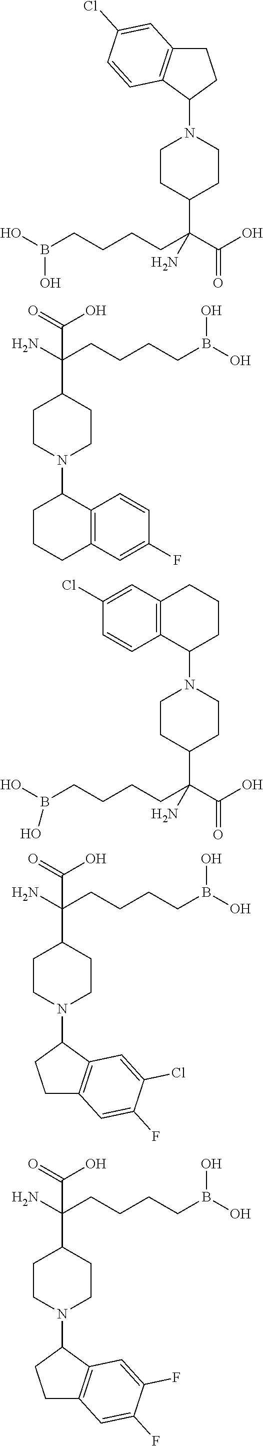

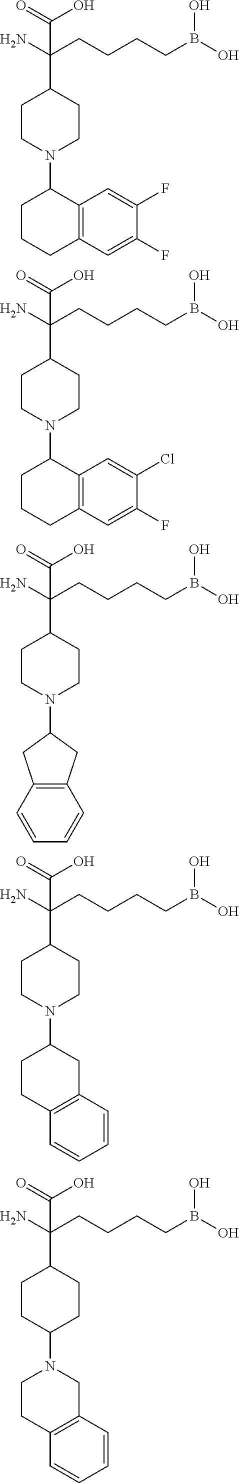

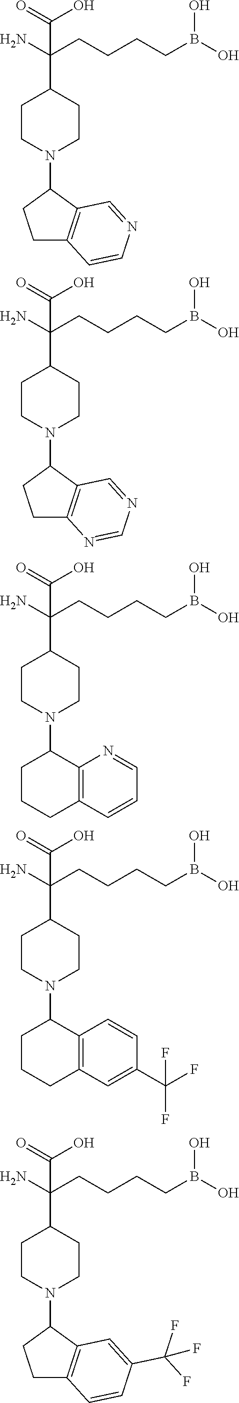

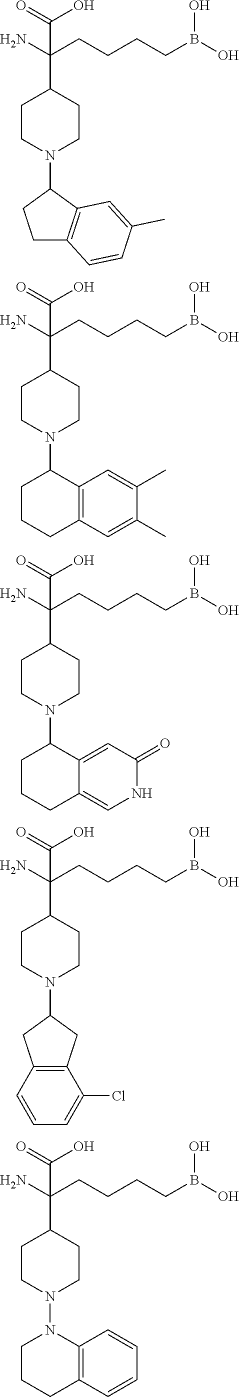

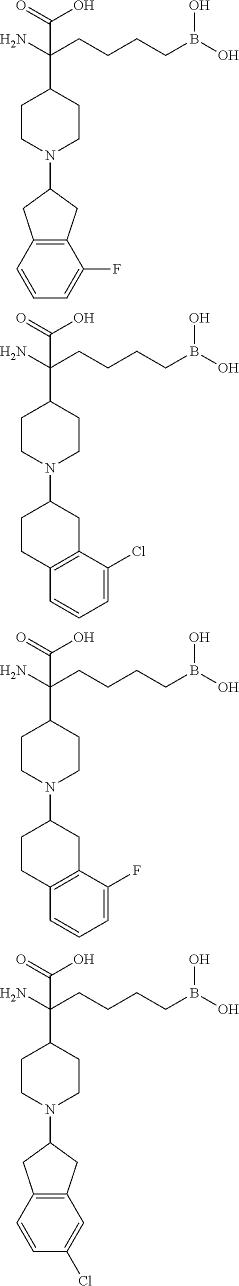

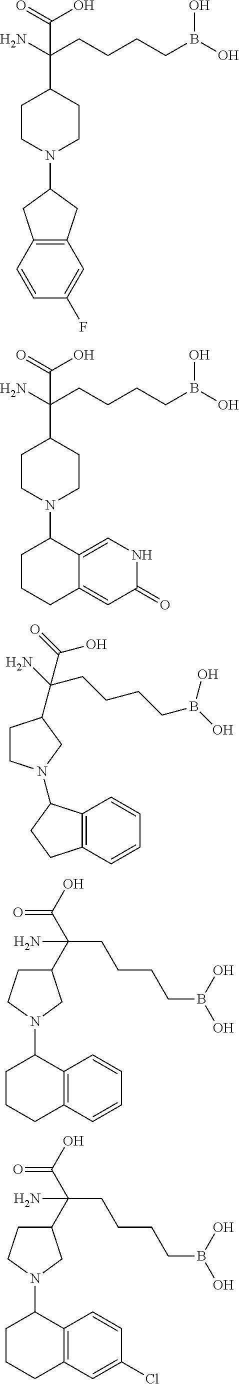

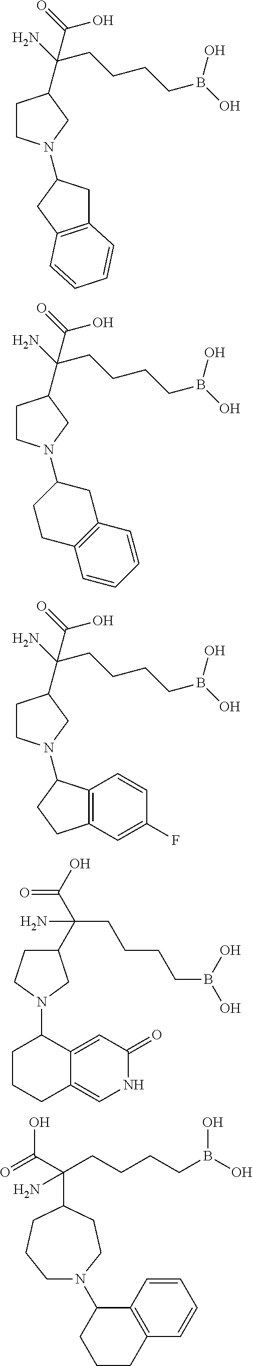

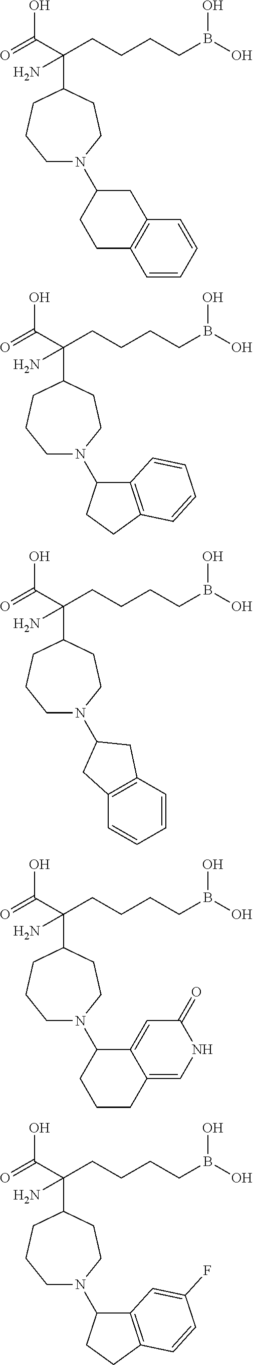

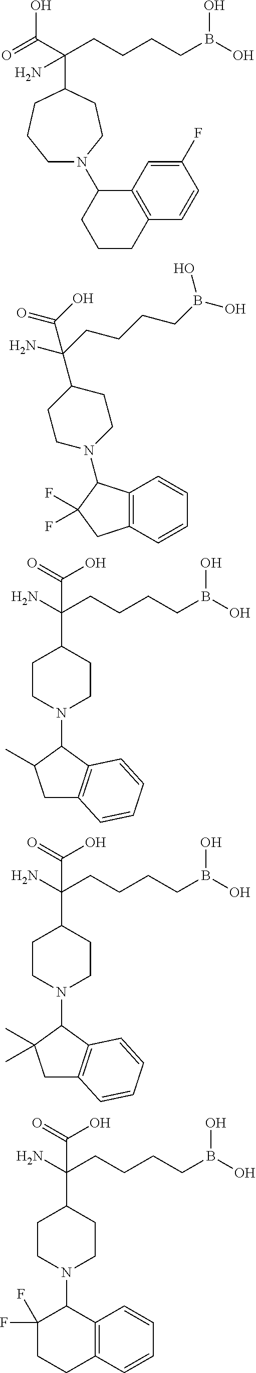

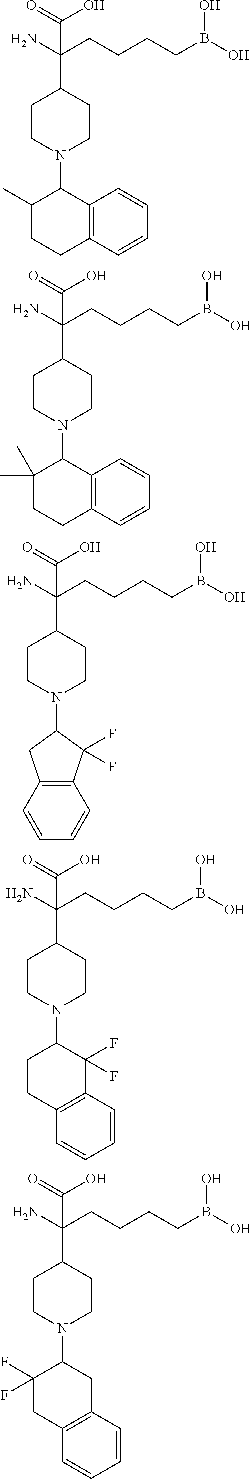

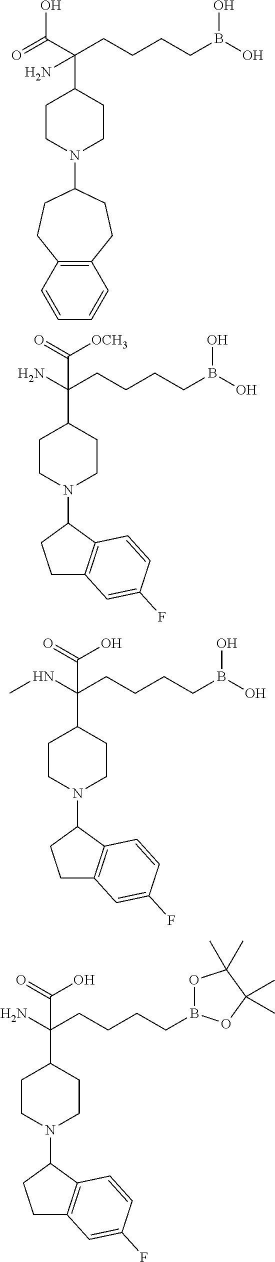

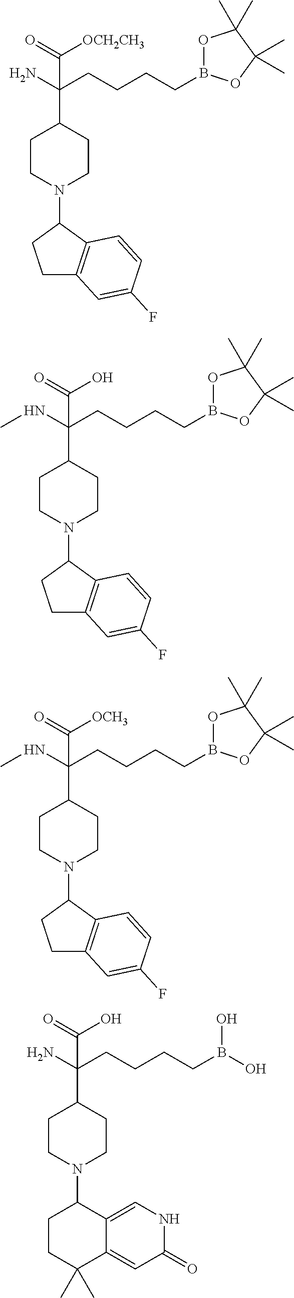

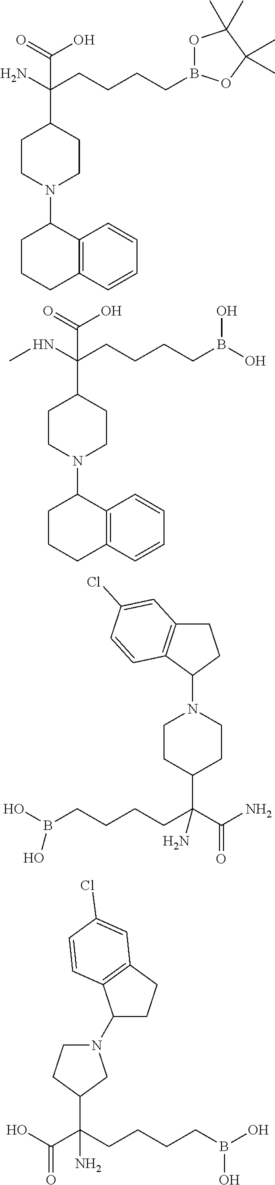

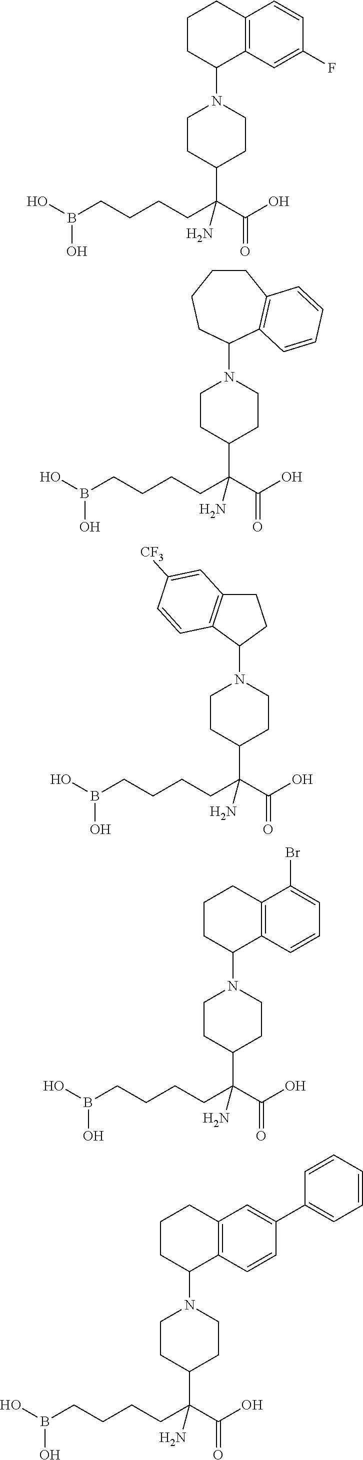

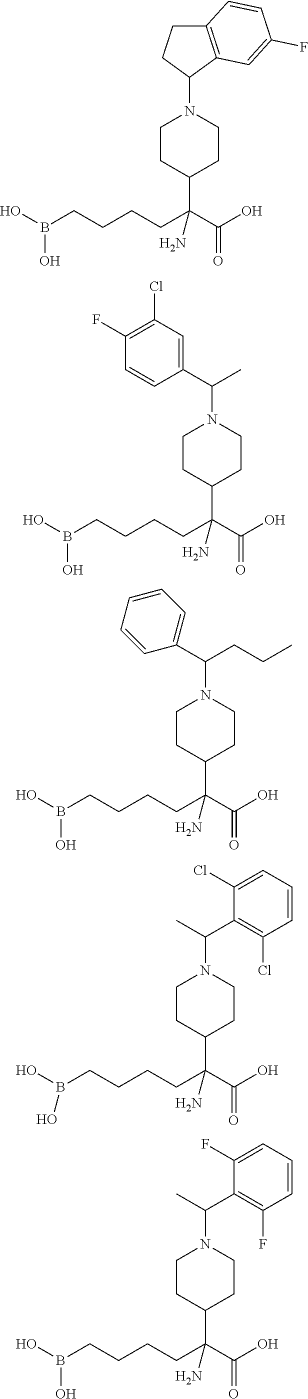

49. The compound of claim 1, or a pharmaceutically acceptable salt, stereoisomer or tautomer thereof, wherein the stereoisomer is selected from the group consisting of

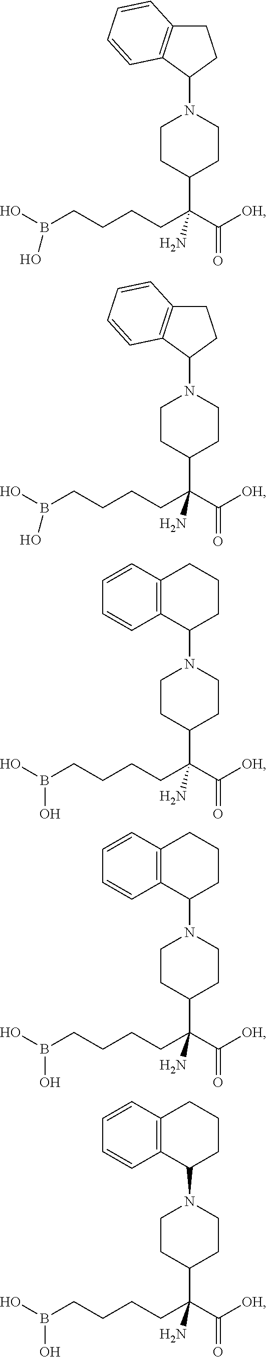

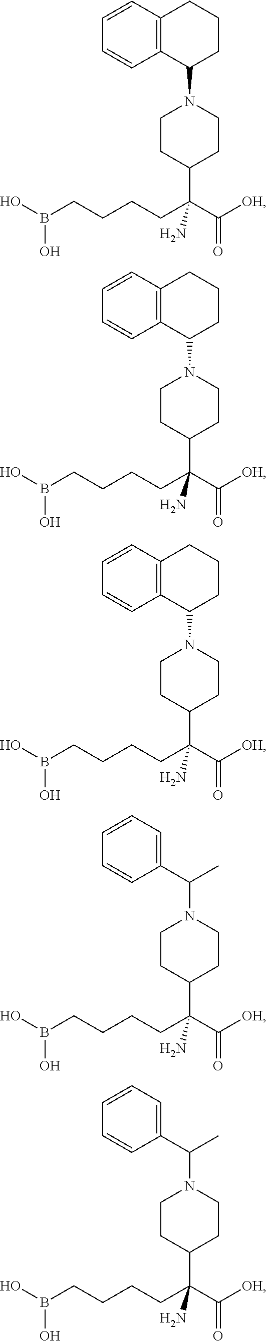

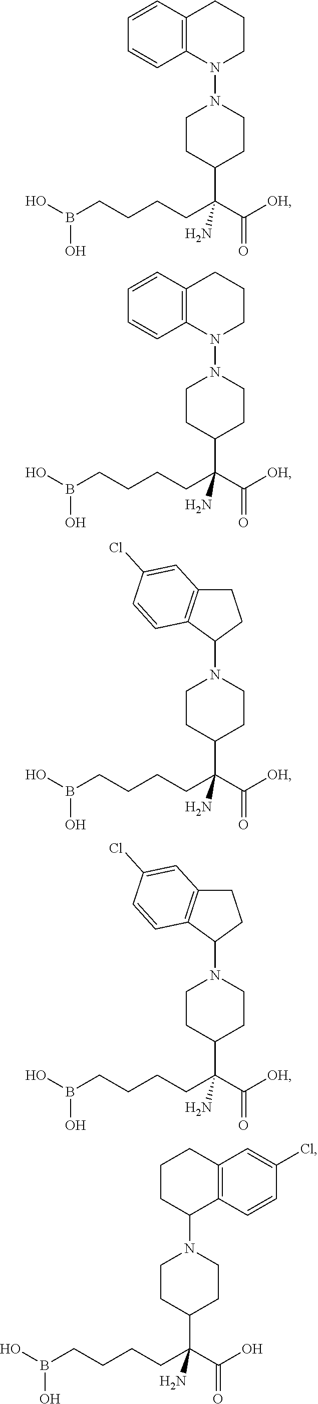

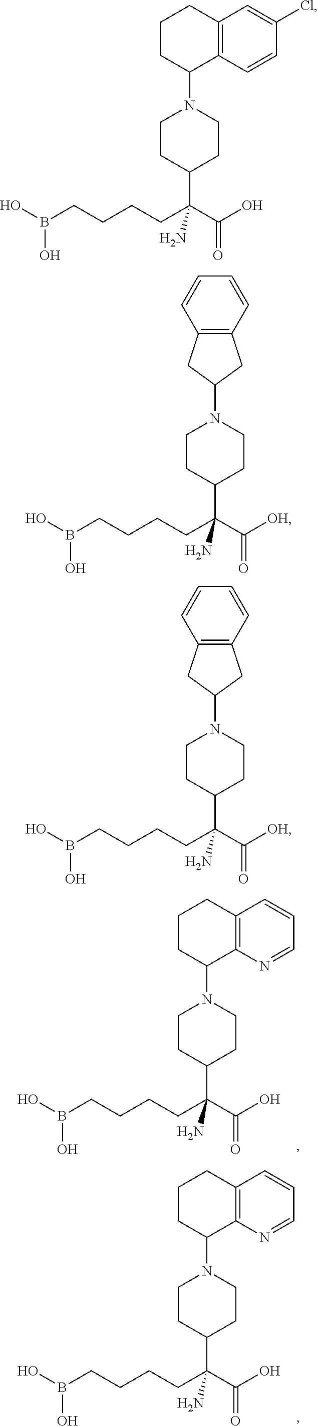

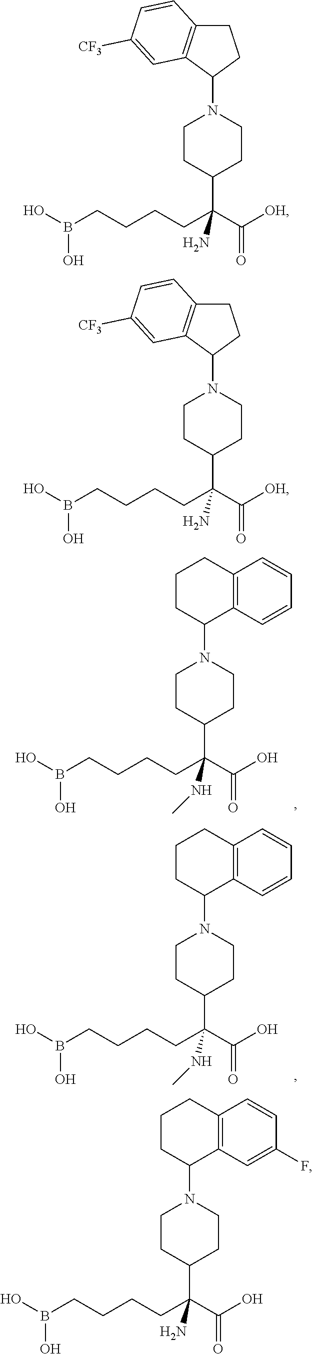

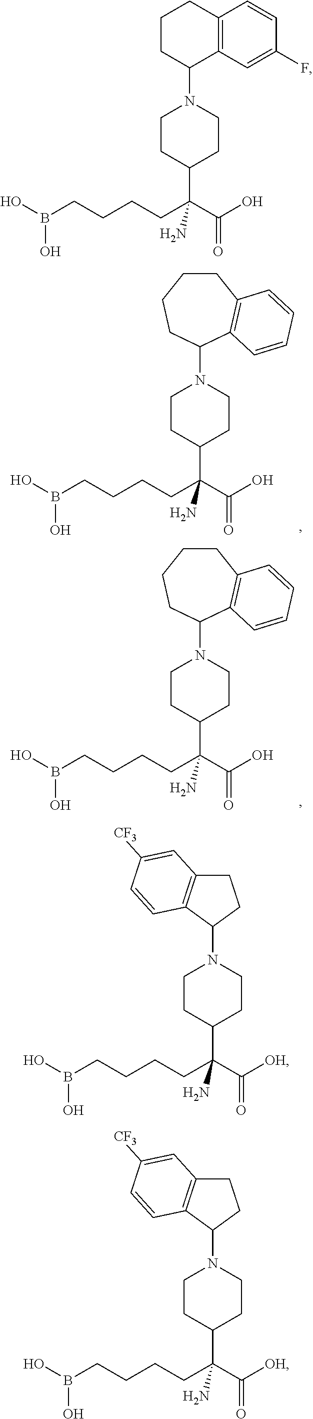

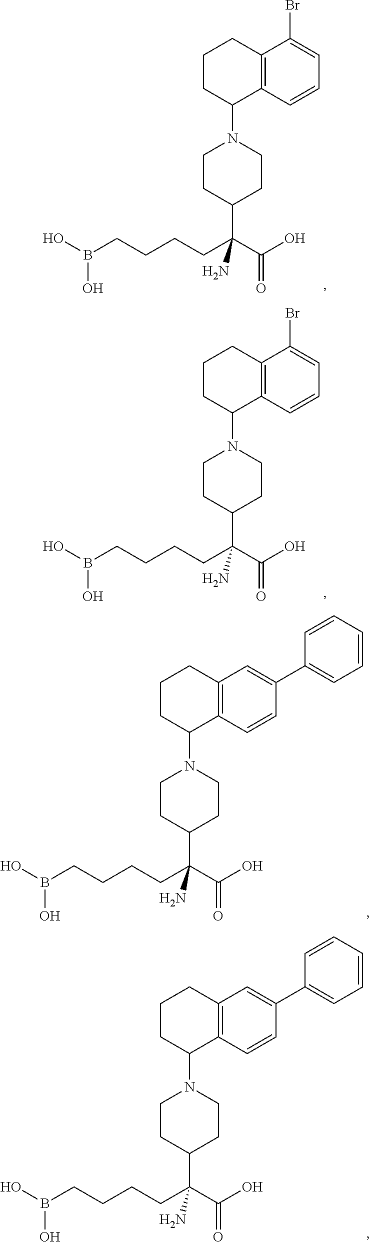

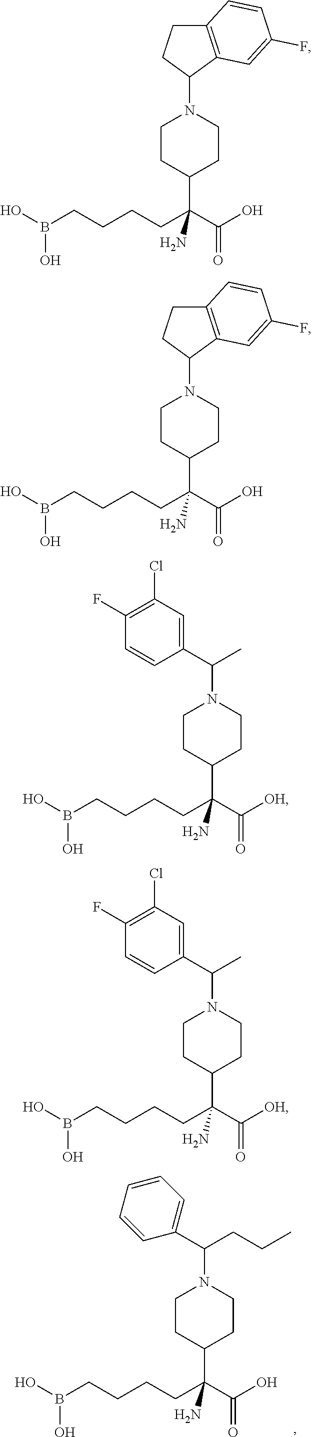

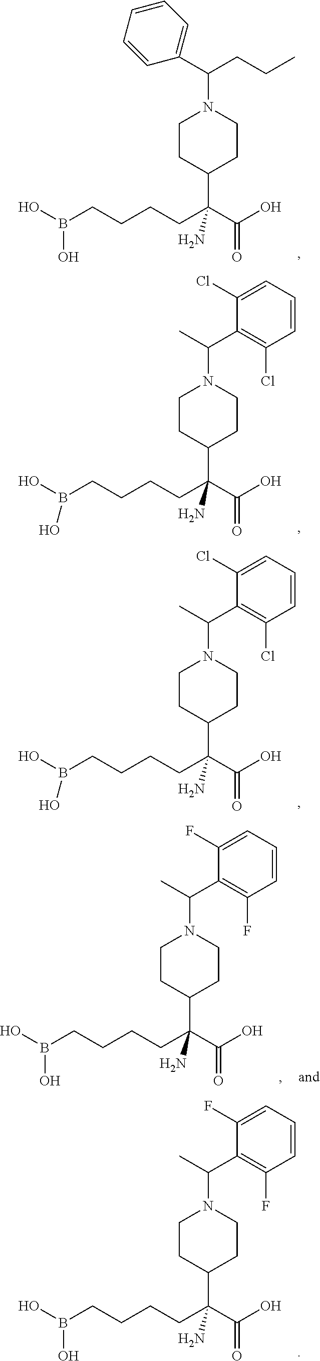

50. A pharmaceutical composition comprising an effective amount of a compound of any one of claims 1-49, or a pharmaceutically acceptable salt, stereoisomer or tautomer thereof, and a pharmaceutically acceptable carrier, diluent, excipient, or combination thereof.

51. A method of inhibiting arginase I, arginase II, or a combination thereof in a cell, comprising contacting the cell with at least one compound according to any one of claims 1-49, a pharmaceutically acceptable salt, stereoisomer or tautomer thereof.

52. A method of the treatment or prevention of a disease or condition associated with expression or activity of arginase I, arginase II, or a combination thereof in an individual in need, comprising administering to the individual a therapeutically effective amount of the compound of any one of claims 1-49, or a pharmaceutically acceptable salt, stereoisomer or tautomer thereof, or the pharmaceutical composition of claim 50.

53. The method of claim 52, wherein the disease or condition is selected from the group consisting of cardiovascular disorders, sexual disorders, wound healing disorders, gastrointestinal disorders, autoimmune disorders, immune disorders, infections, pulmonary disorders and hemolytic disorders.

54. The method of claim 53, wherein the disease or condition is cardiovascular disorder selected from the group consisting of systemic hypertension, pulmonary arterial hypertension (PAH), pulmonary arterial hypertension in high altitude, ischemia reperfusion (IR) injury, myocardial infarction, atherosclerosis.

55. The method of claim 54, wherein the disease or condition is pulmonary arterial hypertension (PAH).

56. The method of claim 54, wherein the disease or condition is myocardial infarction or atherosclerosis.

57. The method of claim 53, wherein the disease or condition is a pulmonary disorder selected from the group consisting of chemically-induced lung fibrosis, idiopathic pulmonary fibrosis, cystic fibrosis, chronic obstructive pulmonary disease (COPD), and asthma.

58. The method of claim 53, wherein the disease or condition is an autoimmune disorder selected from the group consisting of selected from the group consisting of encephalomyelitis, multiple sclerosis, anti-phospholipid syndrome 1, autoimmune hemolytic anaemia, chronic inflammatory demyelinating polyradiculoneuropathy, dermatitis herpetiformis, dermatomyositis, myasthenia gravis, pemphigus, rheumatoid arthritis, stiff-person syndrome, type 1 diabetes, ankylosing spondylitis, paroxysmal nocturnal hemoglobinuria (PNH), paroxysmal cold hemoglobinuria, severe idiopathic autoimmune hemolytic anemia, Goodpasture's syndrome, and systemic lupus erythematosus.

59. The method of claim 53, wherein the disease or condition is an immune disorder selected from the group consisting of myeloid-derived suppressor cell (MDSC) mediated T-cell dysfunction, human immunodeficiency virus (HIV), autoimmune encephalomyelitis, and ABO mismatch transfusion reaction.

60. The method of claim 59, wherein the disease or condition is myeloid-derived suppressor cell (MDSC) mediated T-cell dysfunction.

61. The method of claim 53, wherein the disease or condition is a hemolytic disorder selected from the group consisting of selected from the group consisting of sickle-cell disease, thalassemias, hereditary spherocytosis, stomatocytosis, microangiopathic hemolytic anemias, pyruvate kinase deficiency, infection-induced anemia, cardiopulmonary bypass and mechanical heart valve-induced anemia, and chemical induced anemia.

62. The method of claim 53, wherein the disease or condition is a gastrointestinal disorder selected from the group consisting of gastrointestinal motility disorders, gastric cancers, inflammatory bowel disease, Crohn's disease, ulcerative colitis, and gastric ulcers.

63. The method of claim 53, wherein the disease or condition is a sexual disorder selected from the group consisting of Peyronie's Disease and erectile dysfunction.

64. The method of claim 53, wherein the disease or condition is a wound healing disorder selected from the group consisting of infected and uninfected wound healing.

65. The method of claim 52, wherein the disease or condition is ischemia reperfusion (IR) injury selected from the group consisting of selected from the group consisting of liver IR, kidney IR, and myocardial IR.

66. The method of claim 52, wherein the disease or condition is selected from the group consisting of renal disease inflammation, psoriasis, leishmaniasis, neurodegenerative diseases, wound healing, human immunodeficiency virus (HIV), hepatitis B virus (HBV), H. pylori infections, fibrotic disorders, arthritis, candidiasis, periodontal disease, keloids, adenotonsillar disease, African sleeping sickness and Chagas' disease.

67. A composition for use in any one of the methods of claim 51-66.

68. Use of the compound of any one of claims 1-49, or a pharmaceutically acceptable salt, stereoisomer or tautomer thereof, or the pharmaceutical composition of claim 50 in the manufacture of a medicament for treating or preventing a disease or condition associated with expression or activity of arginase I, arginase II, or a combination thereof in an individual in need.
